SEQUENCE LISTING (46 sequences)

SEQ ID NO: 1 - DNA, 55 bp, Artificial sequence
  Synthetic
  misc_feature (31)..(42): n is a, c, g, or t
catacgagatccgtaatcgggaagctgaagnnnnnnnnnnnngtgagctagtgct

SEQ ID NO: 2 - DNA, 30 bp, Artificial sequence
  Synthetic
catacgagatccgtaatcgggaagctgaag

SEQ ID NO: 3 - DNA, 34 bp, Artificial sequence
  Synthetic
  misc_feature (31)..(34): n is a, c, g, or t
catacgagatccgtaatcgggaagctgaagnnnn

SEQ ID NO: 4 - DNA, 36 bp, Artificial sequence
  Synthetic
  misc_feature (31)..(36): n is a, c, g, or t
catacgagatccgtaatcgggaagctgaagnnnnnn

SEQ ID NO: 5 - DNA, 38 bp, Artificial sequence
  Synthetic
  misc_feature (31)..(38): n is a, c, g, or t
catacgagatccgtaatcgggaagctgaagnnnnnnnn

SEQ ID NO: 6 - DNA, 30 bp, Artificial sequence
  Synthetic
cttcagcttcccgattacggatctcgtatg

SEQ ID NO: 7 - DNA, 12 bp, Artificial sequence
  Synthetic
gcactagctcac

SEQ ID NO: 8 - DNA, 18 bp, Artificial sequence
  Synthetic
gaggctgaggcaggagaa

SEQ ID NO: 9 - DNA, 18 bp, Artificial sequence
  Synthetic
aaaactaaaacaaaaaaa

SEQ ID NO: 10 - DNA, 15 bp, Artificial sequence
  Synthetic
  misc_feature (6)..(6): n is a, c, g, or t
  misc_feature (9)..(9): n is a, c, g, or t
ctaccnccncgccga

SEQ ID NO: 11 - DNA, 14 bp, Artificial sequence
  Synthetic
  misc_feature (6)..(6): n is a, c, g, or t
  misc_feature (9)..(9): n is a, c, g, or t
ctaccnccncacaa

SEQ ID NO: 12 - DNA, 21 bp, Artificial sequence
  Synthetic
ggccatcttggctcctccccc

SEQ ID NO: 13 - DNA, 25 bp, Artificial sequence
  Synthetic
agaagaatgtataactagaataacc

SEQ ID NO: 14 - DNA, 15 bp, Artificial sequence
  Synthetic
  misc_feature (7)..(7): n is a, c, g, or t
  misc_feature (10)..(10): n is a, c, g, or t
ccgcgtnggnggcag

SEQ ID NO: 15 - DNA, 94 bp, Artificial sequence
  Synthetic
  misc_feature (71)..(76): n is a, c, g, or t
cttcagcttcccgattacggatctcgtatgtgtagatctcggtggtcgccgcacgatccgacggtagtgtnnnnnnaaaactaaaacaaaaaaa

SEQ ID NO: 16 - DNA, 91 bp, Artificial sequence
  Synthetic
  misc_feature (71)..(76): n is a, c, g, or t
  misc_feature (82)..(82): n is a, c, g, or t
  misc_feature (85)..(85): n is a, c, g, or t
cttcagcttcccgattacggatctcgtatgtgtagatctcggtggtcgccgcacgatccgacggtagtgtnnnnnnctaccnccncgccga

SEQ ID NO: 17 - DNA, 90 bp, Artificial sequence
  Synthetic
  misc_feature (71)..(76): n is a, c, g, or t
  misc_feature (82)..(82): n is a, c, g, or t
  misc_feature (85)..(85): n is a, c, g, or t
cttcagcttcccgattacggatctcgtatgtgtagatctcggtggtcgccgcacgatccgacggtagtgtnnnnnnctaccnccncacaa

SEQ ID NO: 18 - DNA, 48 bp, Artificial sequence
  Synthetic
  misc_feature (19)..(24): n is a, c, g, or t
  misc_feature (25)..(25): N = generic backbone or linker sequence
  misc_feature (26)..(30): n is a, c, g, or t
tcctacctcaacctcctannnnnnnnnnnnccaaactaaaatacaata

SEQ ID NO: 19 - DNA, 88 bp, Artificial sequence
  Synthetic
  misc_feature (19)..(24): n is a, c, g, or t
  misc_feature (65)..(70): n is a, c, g, or t
tcctacctcaacctcctannnnnncttcagcttcccgattacgggcacgatccgacggtagtgtnnnnnnccaaactaaaatacaata

SEQ ID NO: 20 - DNA, 49 bp, Artificial sequence
  Synthetic
  misc_feature (19)..(24): n is a, c, g, or t
  misc_feature (25)..(25): N = generic backbone or linker sequence
  misc_feature (26)..(31): n is a, c, g, or t
cactacactccaacctaannnnnnnnnnnnncaaaaaactaaaacaaaa

SEQ ID NO: 21 - DNA, 88 bp, Artificial sequence
  Synthetic
  misc_feature (19)..(24): n is a, c, g, or t
  misc_feature (65)..(70): n is a, c, g, or t
cactacactccaacctaannnnnncttcagcttcccgattacgggcacgatccgacggtagtgtnnnnnncaaaaaactaaaacaaaa

SEQ ID NO: 22 - DNA, 49 bp, Artificial sequence
  Synthetic
  misc_feature (19)..(24): n is a, c, g, or t
  misc_feature (25)..(25): N = generic backbone or linker sequence
  misc_feature (26)..(31): n is a, c, g, or t
ttctcctacctcaacctcnnnnnnnnnnnnnccaaactaaaatacaata

SEQ ID NO: 23 - DNA, 88 bp, Artificial sequence
  Synthetic
  misc_feature (19)..(24): n is a, c, g, or t
  misc_feature (65)..(70): n is a, c, g, or t
ttctcctacctcaacctcnnnnnncttcagcttcccgattacgggcacgatccgacggtagtgtnnnnnnccaaactaaaatacaata

SEQ ID NO: 24 - DNA, 49 bp, Artificial sequence
  Synthetic
  misc_feature (19)..(24): n is a, c, g, or t
  misc_feature (25)..(25): N = generic backbone or linker sequence
  misc_feature (26)..(31): n is a, c, g, or t
cactacactccaacctaannnnnnnnnnnnnaaaactaaaacaaaaaaa

SEQ ID NO: 25 - DNA, 88 bp, Artificial sequence
  Synthetic
  misc_feature (19)..(24): n is a, c, g, or t
  misc_feature (65)..(70): n is a, c, g, or t
cactacactccaacctaannnnnncttcagcttcccgattacgggcacgatccgacggtagtgtnnnnnnaaaactaaaacaaaaaaa

SEQ ID NO: 26 - DNA, 20 bp, Artificial sequence
  Synthetic
ccgtaatcgggaagctgaag

SEQ ID NO: 27 - DNA, 20 bp, Artificial sequence
  Synthetic
gcacgatccgacggtagtgt

SEQ ID NO: 28 - DNA, 40 bp, Artificial sequence
  Synthetic
cttcagcttcccgattacgggcacgatccgacggtagtgt

SEQ ID NO: 29 - DNA, 94 bp, Artificial sequence
  Synthetic
  misc_feature (71)..(76): n is a, c, g, or t
cttcagcttcccgattacggatctcgtatgtgtagatctcggtggtcgccgcacgatccgacggtagtgtnnnnnngaggctgaggcaggagaa

SEQ ID NO: 30 - DNA, 96 bp, Artificial sequence
  Synthetic
  misc_feature (19)..(28): n is a, c, g, or t
  misc_feature (69)..(78): n is a, c, g, or t
cactacactccaacctaannnnnnnnnncttcagcttcccgattacgggcacgatccgacggtagtgtnnnnnnnnnncaaaaaactaaaacaaaa

SEQ ID NO: 31 - DNA, 92 bp, Artificial sequence
  Synthetic
cttcagcttcccgattacggatctcgtatgtgtagatctcggtggtcgccgcacgatccgtacggtagtgtggccatcttggctcctccccc

SEQ ID NO: 32 - DNA, 96 bp, Artificial sequence
  Synthetic
cttcagcttcccgattacggatctcgtatgtgtagatctcggtggtcgccgcacgatccgtacggtagtgtagaagaatgtataactagaataacc

SEQ ID NO: 33 - DNA, 86 bp, Artificial sequence
  Synthetic
  misc_feature (78)..(78): n is a, c, g, or t
  misc_feature (81)..(81): n is a, c, g, or t
cttcagcttcccgattacggatctcgtatgtgtagatctcggtggtcgccgcacgatccgtacggtagtgtccgcgtnggnggcag

SEQ ID NO: 34 - DNA, 94 bp, Artificial sequence; identical to SEQ ID NO: 15
  Synthetic
  misc_feature (71)..(76): n is a, c, g, or t

SEQ ID NO: 35 - DNA, 91 bp, Artificial sequence; identical to SEQ ID NO: 16
  Synthetic
  misc_feature (71)..(76): n is a, c, g, or t
  misc_feature (82)..(82): n is a, c, g, or t
  misc_feature (85)..(85): n is a, c, g, or t

SEQ ID NO: 36 - DNA, 90 bp, Artificial sequence; identical to SEQ ID NO: 17
  Synthetic
  misc_feature (71)..(76): n is a, c, g, or t
  misc_feature (82)..(82): n is a, c, g, or t
  misc_feature (85)..(85): n is a, c, g, or t

SEQ ID NO: 37 - DNA, 88 bp, Artificial sequence
  Synthetic
cttcagcttcccgattacggatctcgtatgtgtagatctcggtggtcgccgcacgatccgacggtagtgtgaggctgaggcaggagaa

SEQ ID NO: 38 - DNA, 35 bp, Artificial sequence
  Synthetic
gaggctgaggcaggagaacactgcactccagcctg

SEQ ID NO: 39 - DNA, 35 bp, Artificial sequence
  Synthetic
caggctggagtgcagtgttctcctgcctcagcctc

SEQ ID NO: 40 - DNA, 38 bp, Artificial sequence
  Synthetic
ggatgtatgtggaaggtggggaggctgaggcaggagaa

SEQ ID NO: 41 - DNA, 36 bp, Artificial sequence
  Synthetic
cactgcactccagcctggaagggagggtggtgaagg

SEQ ID NO: 42 - DNA, 38 bp, Artificial sequence
  Synthetic
ttctcctgcctcagcctccccaccttccacatacatcc

SEQ ID NO: 43 - DNA, 36 bp, Artificial sequence
  Synthetic
ccttcaccaccctcccttccaggctggagtgcagtg

SEQ ID NO: 44 - DNA, 20 bp, Artificial sequence
  Synthetic
ggatgtatgtggaaggtggg

SEQ ID NO: 45 - DNA, 19 bp, Artificial sequence
  Synthetic
gaagggagggtggtgaagg

SEQ ID NO: 46 - DNA, 99 bp, Artificial sequence
  Synthetic
  misc_feature (21)..(30): n is a, c, g, or t
  misc_feature (71)..(80): n is a, c, g, or t
cccacgttccacatacatccnnnnnnnnnncttcagcttcccgattacgggcacgatccgacggtagtgtnnnnnnnnnnccttcaccagcctcccttc

(19) United States
(12) Patent Application Publication
(10) Pub. No.: US 2019/0309352 A1
(43) Pub. Date: Oct. 10, 2019

(54) MULTIMODAL ASSAY FOR DETECTING NUCLEIC ACID ABERRATIONS

(71) Applicant: Progenity, Inc, San Diego, CA (US)

(72) Inventors: Jeffrey Buis, Ann Arbor, MI (US); Ronald David Beaubien, JR., Jackson, MI (US); Jay Stoerker, Ann Arbor, MI (US)

(21) Appl. No.: 16/461,211

(22) PCT Filed: Nov. 16, 2017
(86) PCT No.: PCT/US17/61989

§ 371 Date: May 15, 2019

Related U.S. Application Data
(60) Provisional application No. 62/423,179, filed on Nov. 16, 2016.
(60) Provisional application No. 62/451,440, filed on Jan. 27, 2017.

(51) Int. Cl. C12Q 1/6827; C12Q 1/6858; C12Q 1/6806

(52) U.S. Cl. CPC C12Q 1/6827; C12Q 1/6858; C12Q 1/6806; C12Q 2525/151; C12Q 2525/155; C12Q 2523/125; C12Q 2525/307; C12Q 2535/122; C12Q 2563/179; C12Q 2565/514; C12Q 2525/161

(57) ABSTRACT

The invention provides methods for determining whether a subject is predisposed to the disease or condition, or for diagnosing a disease or condition, or for detecting the state of a disease or condition, by detecting nucleic acid fragment size patterns, copy number variations, mutational landscape, genomic instability, methylation status, and combinations thereof in a subject. The invention further provides methods for selecting nucleic acid molecules for use in the methods of the invention.

The present application claims priority to U.S. Provisional Application Ser. No. 62/423,179, filed Nov. 16, 2016, and 62/451,440, filed Jan. 27, 2017, each of which is incorporated herein by reference in its entirety.

### FIELD OF THE INVENTION

This invention relates to systems and methods for determining, inter alia, nucleic acid fragment size patterns, copy number variations, mutational landscape, genomic instability, methylation status, and combinations thereof in a subject.

### BACKGROUND OF THE INVENTION

Genomic aberrations are the hallmark of many diseases and conditions, including cancers, neurodegenerative and neuromuscular diseases, autoimmune and inflammatory conditions, chromosomal abnormalities and metabolic disorders.

Genomic aberration can include point mutations, insertions and deletions, copy number variants, chromosomal translocations and inversions, single- and double-strand breaks in DNA, and gene fusions/rearrangements. In addition, changes in DNA tertiary structure or methylation state can cause genomic instability, the loss of DNA, or the dysregulation of gene expression—all of which can contribute to the onset of disease.

Colorectal cancer is an example of a disease known to have multiple genetic and epigenetic biomarkers. For example, the sequential acquisition of molecular events known to drive “adenoma-to-carcinoma” progression in colorectal cancer includes somatically acquired genomic events like chromosomal gains (13 and 20) and losses (18q and 17p), as well as point mutations and small insertions/deletions in driver genes, such as APC, KRAS, and TP53. (Borras et al. Cancer Prev Res; 9(6) June 2016). Methylation is also known to play a significant role in colorectal cancer, which is characterized by a high frequency of aberrant CpG island methylation. (Lam, Kevin, et al. “DNA methylation based biomarkers in colorectal cancer: A systematic review.” Biochimica et Biophysica Acta (BBA)-Reviews on Cancer 1866.1 (2016): 106-120).

Despite these advances, current methods for nucleic acid analysis, such as whole genome sequencing, require complex workflows and extensive bioinformatics analysis to generate comprehensive genomic profiles. Also, when assaying cell-free samples, such as blood or urine, current methods often cannot establish a biomarker's tissue-of-origin and therefore they require follow-on analysis to pinpoint the location of disease. Therefore, there is a need for developing cost-effective and comprehensive multimodal tests to better capture the heterogeneity and complexity of plasma and genomic nucleic acid biomarkers to improve the detection and treatment of disease.

### SUMMARY OF THE INVENTION

The present invention relates to methods and compositions characterizing nucleic acids of interest, e.g., nucleic acids from a subject, nucleic acids in a sample, etc. Some embodiments comprise:

1. A method for determining the nucleotide sequence of one or more target nucleic acids in a subject comprising:

a) obtaining a nucleic acid sample isolated from a subject;

b) adding an anchor sequence to one of the 3′ or 5′ end of a plurality of nucleic acids from the sample in step a) to create an anchor product;

c) hybridizing an anchor primer to the anchor product of step b), wherein the anchor primer is substantially complementary to the anchor sequence from step b), and hybridizing a genome-informed primer, which is substantially complementary to a repeat sequence in the nucleic acid, to produce a plurality of replicons, wherein the anchor sequence and the repeat sequence flank a gap region in the plurality of target nucleic acid sequences of interest;

d) sequencing a plurality of amplicons that are amplified from the replicons in step c) to determine the nucleotide sequence of one or more target nucleic acids.

2. The method of embodiment 1, wherein the plurality of nucleic acids are single-stranded and the anchor sequence is added via random priming.
3. The method of embodiment 1, wherein the plurality of nucleic acids are double-stranded and the anchor sequence is added via a ligation reaction.
4. The method of embodiments 1-3, wherein the anchor primer is a hybridization anchor arm on a capture probe, and the genome-informed primer is a hybridization genome-informed arm on the opposite end of the same capture probe.
5. The method of embodiment 4, wherein the capture probe is a molecular inversion probe (MIP).
6. The method of embodiment 1, wherein the anchor sequence added to the plurality of nucleic acid sequences in step b) further comprises one or more unique molecular tags. 7. The method of embodiment 1, wherein the anchor sequence added to the plurality of nucleic acid sequences in step b) further comprises one or more linker sequences.
8. The method of embodiment 1, wherein the anchor product of step b), further comprises in sequence the following components:
Anchor sequence—first unique molecular tag—first polynucleotide linker—captured target nucleic acid—second polynucleotide linker.
9. The method of embodiment 4, wherein the capture probe further comprises one or more unique molecular tags.
10. The method of embodiment 4, wherein the capture probe further comprises a backbone sequence.
11. The method of embodiment 9 or 10, wherein the capture probe further comprises in sequence the following components:
Anchor arm—backbone sequence—genome-informed arm.
   12. The method of embodiments 6, 8, 9 or 11 further comprising a method for determining the number of capture events of each of a population of amplicons of the plurality of amplicons provided in step d) by counting the number of the unique molecular tags of each capture probe that produced a replicon, wherein the population of amplicons is determined by the sequence of the target sequence of interest.
   13. The method of embodiment 12 further wherein determining the number of capture events is indicative of a capture bias.
   14. The method of embodiment 12, further comprising using the number of unique molecular tags to identify duplicates to improve analysis.
   15. The method of embodiments 1-14, further comprising determining the number of the unique amplicons sequenced at step d); determining a read density based at least in part on the number of unique amplicon sequences; and detecting copy number variation by comparing the read density to a plurality of reference read densities that are computed based on reference nucleic acid samples isolated from reference subjects.
   16. The method of embodiment 15, further comprising determining the number of unique amplicon sequences in defined regions to determine the read density.
   17. The method of embodiment 3, wherein the plurality of nucleic acids is subjected to end-repair followed by ligation of the anchor sequence to the plurality of nucleic acids in step b) of embodiment 1.
   18. The method of embodiment 3, wherein the plurality of nucleic acids is further subjected to end-repair and phosphorylation followed by ligation of the anchor sequence to the plurality of nucleic acids in step b) of embodiment 1.
   19. The method of embodiment 3, wherein the plurality of nucleic acids is further subjected to end-repair, phosphorylation and A-tailing followed by ligation of the anchor sequence to the plurality of nucleic acids in step b) of embodiment 1.
   20. The method of embodiments 17-19, that further comprises a bead-based cleanup step after the ligation of the anchor sequence to the plurality of nucleic acids in step b) of embodiment 1.
   21. The method of embodiment 1, wherein the method further comprises, before the sequencing step of d), an extension-ligation step to produce circular replicons.
   22. The method of embodiment 1, wherein the method further comprises an exonuclease digestion step that digests non-circular, linear nucleic acids.
   23. The method of embodiment 22, wherein, following the exonuclease digestion, the method further comprises a linearizing step wherein the circular probe is cleaved to become linear.
   24. The method of embodiment 1, wherein the method comprises, before the sequencing step of d), a PCR reaction to amplify the replicons thereby producing amplicons for sequencing.
   25. The method of embodiment 24, wherein the PCR reaction is an indexing PCR reaction.
   26. The method of embodiment 25, wherein the indexing PCR reaction introduces into each of the amplicons the following components: a pair of indexing primers, a unique sample barcode and a pair of sequencing adaptors.
   27. The method of embodiment 26, wherein the barcoded amplicons comprise in sequence the following components:
   a first sequencing adaptor—a first sequencing primer—an anchor arm hybridizing sequence—a first unique molecular tag—a captured target nucleic acid—a genome-informed arm hybridizing sequence—the second unique molecular tag—a unique sample barcode—a second sequencing primer—a second sequencing adaptor.
   28. The method of embodiment 4, wherein the repeat sequence is selected from the group consisting of Alu repeats, protein binding sites, class switch recombination sites, VDJ recombination sites, D4Z4 repeats, centromeric SAT-α repeats, NBL2 repeats, and LINE1 sites.
   29. The method of embodiment 4, wherein the target sequence of interest is located in an Alu element.
   30. The method of embodiment 4, wherein the target sequence of interest is located in the right arm of an Alu element.
   31. The method of embodiment 4, wherein the nucleotide sequence of 50,000 or more different target nucleic acids in a subject is determined using a single capture probe.
   32. The method of embodiment 4, wherein the amplicon sequence from step d) is used to determine the size of the amplicon.
   33. The method of embodiment 32, wherein the size of 1,000 or more different target nucleic acids in a subject is determined using a single capture probe.
   34. The method of embodiment 32, wherein at least some of the nucleic acids are cell-free, target nucleic acids, further comprising:

a) measuring an amount of the amplicons from the sample corresponding to each of a plurality of sizes, the amount including the cell-free, target nucleic acids and background nucleic acids, thereby measuring amounts of nucleic acids at the plurality of sizes;

b) calculating a first value of a first parameter based on the amounts of nucleic acids at the plurality of sizes, the first parameter providing a statistical measure of a size profile of nucleic acids in the sample;

c) comparing the first value to a reference value; and

d) estimating the fractional concentration of the target nucleic acids among background nucleic acid in the sample based on the comparison of step c).

35. The method of embodiment 34, wherein the cell-free target nucleic acid is of apoptotic origin.
36. The method of embodiment 34, wherein the cell-free target nucleic acid is of fetal origin, and the background nucleic acids comprise maternal nucleic acids, whereby the concentration of fetal nucleic acids in a maternal sample is determined.
37. The method of embodiment 36, wherein the reference value is from one or more pregnant subjects with known concentrations of fetal nucleic acids.
38. The method of embodiment 34, wherein the cell-free target nucleic acid is from a tumor, and the background nucleic acids comprise non-tumorigenic nucleic acids, whereby the concentration of tumor nucleic acids in a sample is determined.
39. The method of embodiment 38, the wherein reference value is from one or more cancer-free subjects.
40. The method of embodiment 34, wherein the cell-free target nucleic acid is from a donor, and the background nucleic acids comprise host nucleic acids, whereby the concentration of transplanted donor nucleic acids in a sample is determined.
41. The method of embodiment 4, further comprising determining the number of unique amplicon sequences to measure copy number variation, wherein the number of unique amplicons is determined by the sequence of the target sequence of interest determined in step d).
42. The method of embodiment 41, wherein the number of unique amplicon sequences is compared to a known reference.
43. The method of embodiment 41, further comprising determining the size distribution of each of a population of unique amplicon sequences.
44. The method of embodiment 4, wherein the one or more target nucleic acids from the sample in step a) comprise one or more sequence mutations that are detected by sequencing step d), thereby determining a mutational landscape.
45. The method of embodiment 44, wherein the one or more sequence mutations is selected from the group consisting of single nucleotide variations, deletions, insertions, translocations, fusions, and repeat expansions.
46. The method of embodiment 44, wherein 100 or more different sequence mutations are detected by a single capture probe.
47. The method of embodiment 44, wherein the frequency or type of sequence mutations is compared to a known reference.
48. The method of embodiment 4, wherein a methylation status of one or more target nucleic acids in a subject is determined further comprising:

a) performing bisulfite conversion of the nucleic acid sample;

b) sequencing the bisulfite converted amplicons; and

c) determining the number of occurrences of cytosine nucleotides at each corresponding known CpG site within the unique amplicons, wherein the methylation status is determined based on the number of occurrences of cytosine nucleotides at each corresponding known CpG site.

49. The method of embodiment 1, wherein the nucleosomal occupancy at one or more target nucleic acids in a subject is determined further comprising:

a) hybridizing a genome-informed primer to a protein binding sites in one or more target nucleic acids;

b) determining the size of the plurality of amplicons based on the amplicon sequence from step d), thereby determining an amplicon fragmentation pattern, wherein the fragmentation patterns is indicative of nucleosomal occupancy.

50. The method of embodiment 49, wherein the protein binding site is a transcription factor binding site or a nuclease binding site.
51. The method of embodiment 49, further comprising comparing the nucleosomal occupancy to a reference nucleosomal occupancy.
52. The method of embodiment 1, wherein a gene fusion event at one or more target nucleic acids in a subject is detected further comprising:

a) hybridizing a genome-informed primer to a gene-specific sequence in one or more target nucleic acids;

b) determining the presence or absence of a gene fusion event based on the amplicon sequence from step d), wherein the presence of two different gene sequences in a single amplicon is indicative of a gene fusion event.

53. The method of embodiment 50 or 52, wherein two or more different genome-informed primers are used in a single, multiplexed assay.
54. The method of embodiments 1-53, wherein the nucleic acid sample is DNA or RNA.
55. The method of embodiment 54, wherein the nucleic acid sample is genomic DNA.
56. The method of embodiment 55, wherein the genomic DNA is first fragmented.
57. The method of any one of embodiments 1-55, wherein the blood sample is a whole blood sample, a plasma sample, or a serum sample.
58. The method of embodiment 57, wherein the blood sample is a plasma sample.
59. The method of embodiments 1-58, wherein the sequencing information is used in the determination of whether the subject has the predisposition to the disease or condition, or used in diagnosing the disease or condition, or used in detecting the state of the disease or condition, or used in differentiating the nucleic acid species originating from the subject and the one or more additional individuals.
60. A method of detecting copy number variation in a subject comprising:

a) obtaining a nucleic acid sample isolated from a subject;

b) adding an anchor sequence to one of the 3′ or 5′ end of a plurality of nucleic acid sequences from the sample in step a);

c) capturing a plurality of target sequences of interest in the nucleic acid sample obtained in step a) by using one or more populations of molecular inversion probes (MIPs) to produce a plurality of replicons,

wherein each of the MIPs in the population of MIPs comprises in sequence the following components:

anchor arm—polynucleotide linker—genome-informed arm;

wherein the anchor arm in each of the MIPs is substantially complementary to the anchor sequence from step b), and the genome-informed arm in each of the MIPs is substantially complementary to a repeat sequence in the nucleic acid, such that the anchor sequence and the repeat sequence flank a unique gap region in the plurality of target sequences of interest;

d) sequencing a plurality of MIPs amplicons that are amplified from the replicons obtained in step c);

e) determining the number of a first population of amplicons of the plurality of amplicons provided in step d) based on the number of unique amplicon sequences;

f) determining the number of each of a second population of amplicons of the plurality of amplicons provided in step d) based on the number of unique amplicon sequences;

g) determining, for each target sequence of interest from which the first population of amplicons was produced, a site capture metric based at least in part on the number of capture events determined in step e);

h) identifying a first subset of the site capture metrics determined in step g) that satisfy at least one criterion;

i) determining, for each target sequence of interest from which the second population of amplicons was produced, a site capture metric based at least in part on the number of capture events determined in step f);

j) identifying a second subset of the site capture metrics determined in step i) that satisfy the at least one criterion;

k) normalizing a first measure determined from the first subset of site capture metrics identified in step h) by a second measure determined from the second subset of site capture metrics identified in step j) to obtain a test ratio;

l) comparing the test ratio to a plurality of reference ratios that are computed based on reference nucleic acid samples isolated from reference subjects without a copy number variation at the target sequences of interest; and

m) determining, based on the comparing in step 1), whether a copy number variation is present at the target sequences of interest in a subject.

61. The method of embodiment 60, wherein the nucleic acid sample is isolated from a maternal blood sample comprising fetal nucleic acid, and the copy number variation is a fetal aneuploidy determined by comparing the test ratio to a plurality of reference ratios that are computed based on reference nucleic acid samples isolated from reference subjects known to exhibit euploidy or aneuploidy.
62. The method of embodiment 60, wherein the nucleic acid sample is isolated from a maternal blood sample comprising fetal nucleic acid, and a fetal aneuploidy is detected further comprising:

comparing the distribution of maternal and fetal amplicon sequences from the maternal sample to the normal distribution of amplicon sequences from a euploid chromosome from the same sample, whereby a chromosomal copy number variation is indicative of a fetal aneuploidy.

63. The method of embodiment 60 or 61, wherein the size of one or more amplicons from the plurality of target sequences is determined based on the amplicon sequence from step d).
64. The method of any one of embodiments 60-63, wherein the site capture metric is a site capture efficiency index (SCE).
65. The method of any one of embodiments 60-64, wherein the site capture metric is a site capture consistency measure (SCC).
66. The method of any one of embodiments 60-65, wherein each of the MIPs replicons provided in step c) is produced by:

i) the anchor arms and genome-informed arms, respectively, hybridizing to the first and second regions in the nucleic acid sample, respectively, wherein the first and second regions flank a target sequence of interest; and

ii) after the hybridization, using a ligation/extension mixture to extend and ligate the gap region between the two arms to form single-stranded circular nucleic acid molecules.

67. The method of any one of embodiments 60-66, wherein the method comprises, before the sequencing step of d), a PCR reaction to amplify the MIPs replicons for sequencing.
68. The method of embodiment 67, wherein the PCR reaction is an indexing PCR reaction.
69. The method of embodiment 68, wherein the indexing PCR reaction introduces into each of the MIPs amplicons the following components: a pair of indexing primers, a unique sample barcode and a pair of sequencing adaptors.
70. The method of embodiment 69, wherein the barcoded MIPs amplicons comprise in sequence the following components:
a first sequencing adaptor—a first sequencing primer—the first unique targeting molecular tag—the anchor arm arm—captured nucleic acid—the genome-informed arm—the second unique targeting molecular tag—a unique sample barcode—a second sequencing primer—a second sequencing adaptor.
71. The method of any one of embodiments 60-70, wherein the first plurality of target sequences of interest is on a single chromosome.
72. The method of any one of embodiments 60-71, wherein the second plurality of target sequences of interest are on multiple chromosomes.
73. The method of embodiment 63, wherein at least some of the nucleic acids are cell-free, target nucleic acids, further comprising:

a) measuring an amount of the amplicons from the sample corresponding to each of a plurality of sizes, the amount including the cell-free, target nucleic acids and background nucleic acids, thereby measuring amounts of nucleic acids at the plurality of sizes;

b) calculating a first value of a first parameter based on the amounts of nucleic acids at the plurality of sizes, the first parameter providing a statistical measure of a size profile of nucleic acids in the sample;

c) comparing the first value to a reference value; and

d) estimating the fractional concentration of the target nucleic acids among background nucleic acid in the sample based on the comparison of step c).

74. The method of embodiment 73, wherein the cell-free target nucleic acid is of fetal origin, and the background nucleic acids comprise maternal nucleic acids, whereby the concentration of fetal nucleic acids in a maternal sample is determined.
75. The method of embodiment 74, wherein the reference value is from one or more pregnant subjects with known concentrations of fetal nucleic acids.
76. The method of embodiment 73, wherein the cell-free target nucleic acid is from a tumor, and the background nucleic acids comprise non-tumorigenic nucleic acids, whereby the concentration of tumor nucleic acids in a sample is determined.
77. The method of embodiment 76, wherein the reference value is from one or more cancer-free subjects.
78. The method of embodiment 73, wherein the cell-free target nucleic acid is from a donor, and the background nucleic acids comprise host nucleic acids, whereby the concentration of transplanted donor nucleic acids in a sample is determined.
79. A method of determining the methylation status of one or more nucleic acid fragments in a subject comprising:

a) obtaining a nucleic acid sample isolated from a subject;

b) performing bisulfite conversion of the nucleic acid sample;

c) adding an anchor sequence to the bisulfite-converted nucleic acid of step b);

d) capturing a plurality of target sequences of interest in the nucleic acid sample obtained in step a) by using one or more populations of molecular inversion probes (MIPs) to produce a plurality of replicons,

wherein each of the MIPs in the population of MIPs comprises in sequence the following components:

anchor arm—polynucleotide linker—genome-informed arm;

wherein the anchor arm in each of the MIPs is substantially complementary to the anchor sequence from step c), and the genome-informed arm in each of the MIPs is substantially complementary to a repeat sequence in the nucleic acid, such that the anchor sequence and the repeat sequence flank a unique gap region in the plurality of target sequences of interest;

e) sequencing a plurality of MIPs amplicons that are amplified from the replicons obtained in step d);

f) determining the number of occurrences of cytosine nucleotides at each corresponding known CpG site within the MIPs amplicons sequenced at step e), wherein the methylation status is determined based on the number of occurrences of cytosine nucleotides at each corresponding known CpG site.

80. The method of embodiment 79, wherein the size of one or more amplicons from the plurality of target sequences is determined based on the amplicon sequence from step d).
81. The method of embodiment 80, wherein at least some of the nucleic acids are cell-free, target nucleic acids, further comprising:

a) measuring an amount of the amplicons from the sample corresponding to each of a plurality of sizes, the amount including the cell-free, target nucleic acids and background nucleic acids, thereby measuring amounts of nucleic acids at the plurality of sizes;

b) calculating a first value of a first parameter based on the amounts of nucleic acids at the plurality of sizes, the first parameter providing a statistical measure of a size profile of nucleic acids in the sample;

c) comparing the first value to a reference value; and

d) estimating the fractional concentration of the target nucleic acids among background nucleic acid in the sample based on the comparison of step c).

82. The method of embodiment 79, wherein the methylation status of step e), is compared to a reference value.
83. The method of embodiment 82, wherein the reference value is a methylation status from a specific tissue type.
84. The method of embodiment 83, wherein the reference value is a methylation status from a diseased tissue type.
85. A method for characterizing one or nucleic acids of interest from a subject, comprising:

a) obtaining a nucleic acid sample isolated from a subject;

b) adding clip sequences to the 3′ and 5′ ends of each of a plurality of target nucleic acids from the sample in step a) to create a clip product, wherein the two clip sequences flank a gap region in the target nucleic acid sequence of interest;

c) hybridizing a capture probe comprising two clip binding arms to the clip product of step b), wherein the two clip binding arms are on opposite ends of the same capture probe, and wherein each clip binding arm is substantially complementary one of the clip sequences from step b);

d) using a ligation/extension mixture to extend and ligate the gap region between the two clip binding arms to form a single-stranded circular nucleic acid molecule; and

e) analyzing a plurality of amplicons that are amplified from single-stranded circular nucleic acid molecules of step d) to characterize the one or more nucleic acids of interest.

86. The method of embodiment 85, wherein analyzing a plurality of amplicons of step e) comprises determining one or more of size, size distribution, nucleotide sequence, and/or amounts one or more of said plurality of amplicons.
87. The method of embodiment 85 or 86, wherein amplifying the plurality of amplicons from single-stranded circular nucleic acid molecules comprises a polymerase chain reaction.
88. The method of any one of embodiments 85-87, wherein the plurality of target nucleic acids are double-stranded, and wherein one or both clip sequences are added by ligation.
89. The method of embodiment 88, wherein the double-stranded plurality of nucleic acids is subjected to one or more of end-repair, phosphorylation, and A-tailing prior to ligation of said one or both clip sequences.
90. The method of any one of embodiments 85-89, wherein the clip sequences added in step a) are added using target-specific adaptor oligonucleotides, wherein the target-specific adapter oligonucleotides comprise a sequence substantially complementary to a clip arm and a sequence substantially complementary to a 5′ or 3′ terminal portion of a target nucleic acid sequence of interest.
91. The method of any one of embodiments 85-90, further comprising prior to step c) a step of treating the clip product with bisulfite under conditions wherein unmethylated cytosines are converted to uracils.
92. The method of embodiment 91, wherein the clip sequences added in step b) do not comprise cytosines.
93. The method of any one of embodiments 85-92, wherein the method further comprises an exonuclease digestion step that digests non-circular, linear nucleic acids.
94. The method of embodiment 93, wherein, following the exonuclease digestion, the method further comprises a linearizing step wherein the single-stranded circular nucleic acid molecule is cleaved to become linear.
95. The method of any one of embodiments 86-94, wherein nucleotide sequences of at least 50,000 different nucleic acids from the subject are determined using a single capture probe.
96. The method of any one of embodiments 86-95, wherein the sizes of at least 1,000 different nucleic acids in the nucleic acid sample are determined using a single capture probe.

### BRIEF DESCRIPTION OF THE DRAWINGS

FIG. 1 shows the addition of anchor sequences to fragmented, double-stranded target nucleic acid via ligation. In some embodiments, target nucleic acid undergoes end-repair, clean-up and A-tailing to prepare the target for subsequent ligation of the anchor sequences to the target. In some methods of this disclosure, linker sequences and unique molecular identifiers (labeled UMID₁ and UMID₂) are also ligated to the target nucleic acid sequence as depicted in FIG. 1. The unique molecular identifiers (or tags) are generally random polynucleotide sequences. The resulting molecule is referred to as a ligation product, which is a type of anchor product created via a ligation reaction.

FIG. 2 shows an embodiment for capturing and amplifying anchor products using amplification primers. An anchor primer binds to an anchor sequence of the anchor product, while a genome-informed primer binds to a repeat sequence found in the target nucleic acid region of the anchor product. The primers amplify the anchor product, which can be subsequently sequenced.

FIG. 3 shows an embodiment for capturing anchor products using a capture probe. The capture probe depicted in FIG. 3 is a molecular inversion probe (MIP), which comprises in sequence the following components: an anchor arm, a polynucleotide linker (labeled “MIP Backbone,” and genome-informed arm. The anchor arm and genome-informed arm in each of the MIP are substantially complementary to anchor sequences and repeat sequences in the anchor product that, respectively, flank a site of interest. In some embodiments, “substantially complementary” refers to 0 mismatches in both arms, or at most 1 mismatch in only one arm (e.g., when the targeting polynucleotide arms hybridize to the first and second regions in the nucleic acid that, respectively, flank a site of interest). In some embodiments, “substantially complementary” refers to at most a small number of mismatches in both arms, such as 1, 2, 3, 3, 5, 6, 7, or 8. Following hybridization, a polymerase and a ligase are added under extension/ligation conditions, and a circular oligonucleotide (the “replicon”) is produced by DNA synthesis across the target sequence of interest containing the unique gap sequence between the anchor and genome-informed arms. Depending on the location of the repeat sequence in the target nucleic acid, the gap sequence of the replicon will be of varying sizes or lengths. Upon melting of the amplicon and the anchor product, the replicon is ready for amplification.

FIG. 4 shows amplification of the replicon described in FIG. 3 using indexing PCR. Nucleic acid molecules comprising a sequencing adapter and a forward or a reverse PCR primer bind to the backbone of the replicon, and amplify the replicon using PCR. The amplicons are then sequenced using, for example, next generation sequencing (NGS), and the read count for the resulting amplicons is determined by counting the number of occurrences of the unique molecular tags in each amplicon

FIG. 5 shows a population of amplicons ready for sequencing and subsequent analysis, including mutational landscaping and copy number analysis. As illustrated, the amplicons can be of varying size depending on the gap sequence length, which allows for fragment size distribution analysis. The unique molecular identifier can count individual capture event which can be used for copy number analysis, while the sequencing barcode can allow for, inter alia, sample multiplexing.

FIG. 6 shows an exemplary genome-informed arm design, which can bind to fixed regions of repeats found in a genome. FIG. 6 shows the partial structure of an Alu element with the corresponding genome-informed binding location in the fixed region. As shown in FIGS. 5 and 6, the inherent fragmentation size pattern of cell-free DNA (cfDNA) can be captured using the compositions and methods described herein.

FIG. 7 shows an exemplary genome-informed arm design for capturing and detecting target nucleic acids comprising CCCTC-binding factor (CTCF) motifs or other transcription factor binding sites. The fragmentation size pattern of cfDNA from the binding motif site can be used to detect level of nucleosome occupancy at these sites across the genome. An exemplary CTCF consensus sequence is provided in the Figure. [Move this to the later Example: Example probe for Nucleosomal Occupancy Analysis: 5′-/SPHOS/CTT CAG CTT CCC GAT TAC GGA TCT CGT ATG TGT AGA TCT CGG TGG TCG CCG CAC GAT CCG ACG GTA GTG TCT GCC NCC NCG CGG-3′]

FIG. 8 shows an exemplary genome-informed arm design for capturing and detecting V(D)J recombination, transcription and splicing events, which can be useful for genome-wide immune repertoire analysis (i.e., immune diversity and maturity analysis of antibodies). More specifically, the variable region of immunoglobulin (Ig) heavy chain is encoded by three separate genes: variable (VH), diversity (DH) and joining (JH) genes on the germ-line genome, which can be detected and quantified using the compositions and methods described herein.

FIG. 9 shows a genome-informed arm design for capturing and detecting gene fusion events, which can be useful for cancer diagnosis, prognosis and treatment. The Figure shows a TMPRSS2-ERG (ETV1, ETV4, ETV5) gene fusion event that can be found in solid tumors such as prostate cancer. Gene fusions typically occur with a diversity of break points, leaving targeted molecular detection difficult. The compositions and methods described herein allow for the detection of said gene fusion events using modified genome-informed arm sequences that target genes known to form chimeric gene fusions. As shown in FIG. 9, the genome-informed arms can be designed to tile across fusion gene breakpoints, and the resulting amplicons can be sequenced to detect target nucleic acids comprising multiple genes, and the unique molecular identifiers in the amplicon can be used to count the individual capture events. For gene fusion events, MIPs may need to be multiplexed, wherein the genome-informed arms are designed to capture areas of the genome known to be susceptible to breakage or rearrangement (e.g., hot spot recombination sites).

FIGS. 10A-B show the distribution of amplicons across different nucleic acid fragments lengths. The library of replicons retains the fidelity of the starting, sheared genomic DNA pattern—though shifted in size due to the addition of the adaptors; thereby confirming the assay's performance.

FIG. 11 shows an embodiment for determining the tissue-of-origin of cfDNA using the compositions and methods described herein. In one embodiment, a FireMIP can be designed to target transcription factor binding sites (See FIG. 7). By capturing and sequencing amplicons as described herein, cfDNA of different size lengths from plasma DNA or other sources can be used to generate fragmentation patterns. A typical Gaussian size distribution across the target nucleic acids is expected if no other forces are influencing the target nucleic acid (i.e., the target transcription factor binding site). Perturbations to the Gaussian can be detected, and indicate the presence of non-random influences on the target nucleic acids (e.g., the presence of nucleosomal occupancy). In samples from differing tissues, the nucleosomal occupancy of these sites can be different and is dependent on a host of binding proteins. Therefore, after determining the nucleosomal occupancy of these sites by looking for perturbations from the Gaussian distributions, one can compare an unknown sample to the known distribution patterns and match the pattern of the unknown sample to the known references. As tissue type has orders of magnitude greater effects on this occupancy than population variations, the difference found among individuals will be minimal.

FIG. 12 shows an embodiment for determining the methylation status of a nucleic acid sample using the compositions and methods described herein. In one embodiment, bisulfite conversion of target nucleic acids is followed by the addition of anchor sequences through random priming. Bisulfite conversion can be done using known methods and reagents, such as the Zymo EZ-Methylation Gold Kit. The resulting bisulfite converted single-stranded DNA is subjected to random priming, which incorporates anchor sequences into the resulting amplification product. In some embodiments outside of methylation analysis, random priming represents another method to incorporate anchor sequences into target nucleic acids to create anchor products. As used herein, “anchor products” can be interchanged with “ligation products” to capture other ways for adding anchor sequences to nucleic acids.

FIG. 13 is an illustrative embodiment of a computing device for performing any of the processes as described in accordance with the methods of the invention.

FIG. 14 is a representative process flow diagram for designing and selecting a probe according to the methods of the invention.

FIG. 15 is a representative process flow diagram for predicting a methylation state in a test subject according to the methods of the invention.

FIG. 16 is another representative and more detailed process flow diagram for predicting a disease state of a test subject according to the methods of the invention.

FIG. 17 shows the addition of Clip sequences to fragmented, double-stranded target nucleic acid via hybridization and ligation. In some embodiments, the target nucleic acid undergoes end-repair, clean-up. In some embodiments, linker sequences and unique molecular identifiers are also included as part of the Clip sequences. For subsequent methylation analysis, the Clip sequences are designed not to contain cytosines to enable bisulfite treatment in later steps.

FIG. 18 shows an embodiment for capturing Clip products using a capture probe. The capture probe depicted in the Figure is a MIP, which hybridizes to the target nucleic acid containing Clip sequences. Following hybridization, a polymerase and a ligase are added under extension/ligation conditions, and a circular oligonucleotide (the “replicon”) is produced by DNA synthesis across the target sequence of interest containing the unique gap sequence.

FIG. 19 shows exemplary Clip sequences that can be incorporated into a target nucleic acid from an Alu repeat region.

FIG. 20 shows an exemplary ClipMIP sequence for capturing target nucleic acid modified to include Clip sequences.

### DETAILED DESCRIPTION OF THE INVENTION

The invention provides a system and method for detecting diseases or conditions. There is a need for informative, non-invasive tools facilitating improved diagnosis, prognosis, and surveillance of human disease. Several complex diseases including carcinogenesis display altered genomic profiles, including different methylation patterns, genomic instability, altered genomic landscapes or genomic rearrangements. To this end, the inventors have developed a single-probe capture method for sequencing ready libraries from input of DNA as low as 200 pg of tissue or circulating genetic material. This method simultaneously assesses>200,000 sites across the genome, which can be analyzed simultaneously to generate a genomic profile while also capturing nucleic acid size information which is useful for differentiating nucleic acid species.

This disclosure provides a system and method, herein referred to as Fixed Fractional Repeat Element Sequencing (FireMIP), for generating a comprehensive genomic profile in a single assay. The genomic profile may comprise one or more of nucleic acid sequence information, nucleic acid size distribution, the presence or absence of nucleic acid copy number variations, the presence or absence of genomic rearrangements including gene fusion events, mutational landscape analysis and methylation analysis—all of which can be detected simultaneously in a single assay or a multiplexed assay in the case of gene fusion events. The methods and compositions disclosed herein may be useful for the detection, diagnosis, or prognosis of a wide range of diseases and conditions including, but not limited to, cancer, pregnancy-related disorders, neurodegenerative and neuromuscular diseases, autoimmune and inflammatory conditions, chromosomal abnormalities and metabolic disorders, and detecting the recurrence or minimum residual risk of any of the above. The methods and compositions disclosed herein may be also be useful for characterizing the genome, for example, analyzing an immune repertoire.

The following detailed description is set forth as an aid to understanding various embodiments.

Unless otherwise defined herein, scientific and technical terms used in this application shall have the meanings that are commonly understood by those of ordinary skill in the art to which this invention belongs. Generally, nomenclature used in connection with, and techniques of, cell and tissue culture, molecular biology, cell biology, cancer biology, neurobiology, neurochemistry, virology, immunology, microbiology, genetics, protein and nucleic acid chemistry, chemistry, and pharmacology described herein, are those well-known and commonly used in the art. Each embodiment of the inventions described herein may be taken alone or in combination with one or more other embodiments of the inventions.

The methods and techniques of the various embodiments are generally performed, unless otherwise indicated, according to methods of molecular biology, cell biology, biochemistry, microarray and sequencing technology well known in the art and as described in various general and more specific references that are cited and discussed throughout this specification. See, e.g. Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); Gilbert et al., “Developmental Biology, 6th ed.”, Sinauer Associates, Inc., Sunderland, Mass. (2000).

Chemistry terms used herein are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to”. “Including” and “including but not limited to” are used interchangeably.

The following terms and definitions are provided herein.

### Definitions

The term “DNA methylation” as used herein refers to the addition of a methyl group to the 5′ carbon of cytosine residues (i.e. 5-methylcytosines) among CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported.

The term “methylation state” or “methylation status” as used herein, refers to the state of a nucleic acid molecule or population of nucleic acid molecules with respect to the methylation of certain nucleotides. For example, genomic DNA is methylated at certain sites (e.g., CpG sites) at cytosine nucleotides. Thus, the methylation state of a nucleic acid may refer to the ratio of CpG sites in a genome that is methylated, or the ratio of CpG sites in a genome that is unmethylated.

The term “methylation score” as used herein refers to a ratio calculated from the number of cytosine sites (C's) observed at CpG sites. It may also be referred to as a “test ratio” or “reference ratio”. The methylation score provides the ratio of unconverted (i.e., methylated) cytosine nucleotides at one or more CpG sites usually across a region or the entire genome. The methylation score can be calculated using the following ratio: Methylated C's at CpG site/(Methylated C's at CpG site+Unmethylated C's at CpG site)

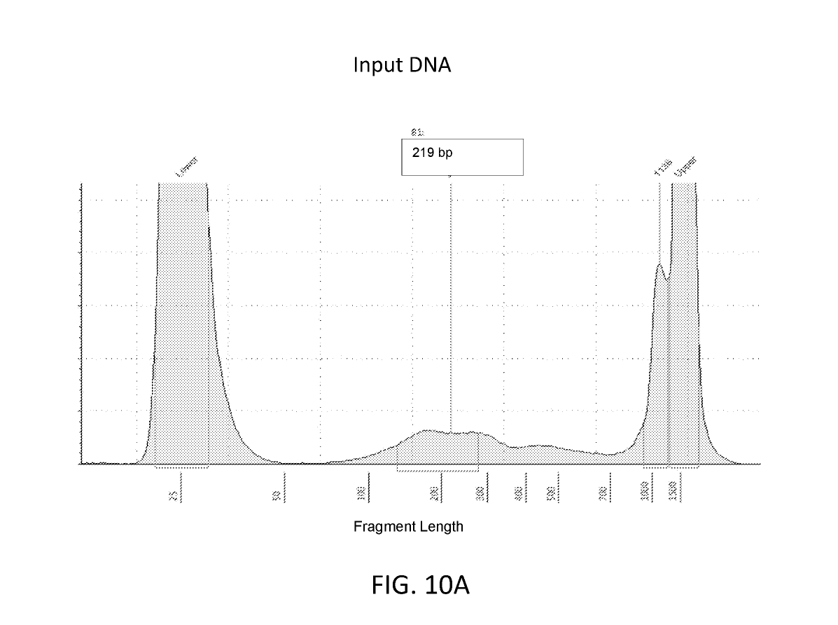

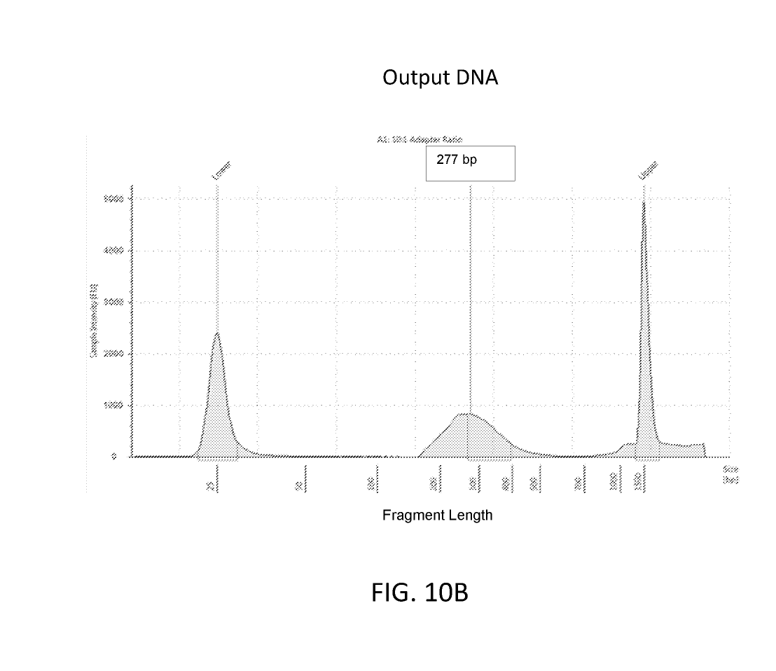

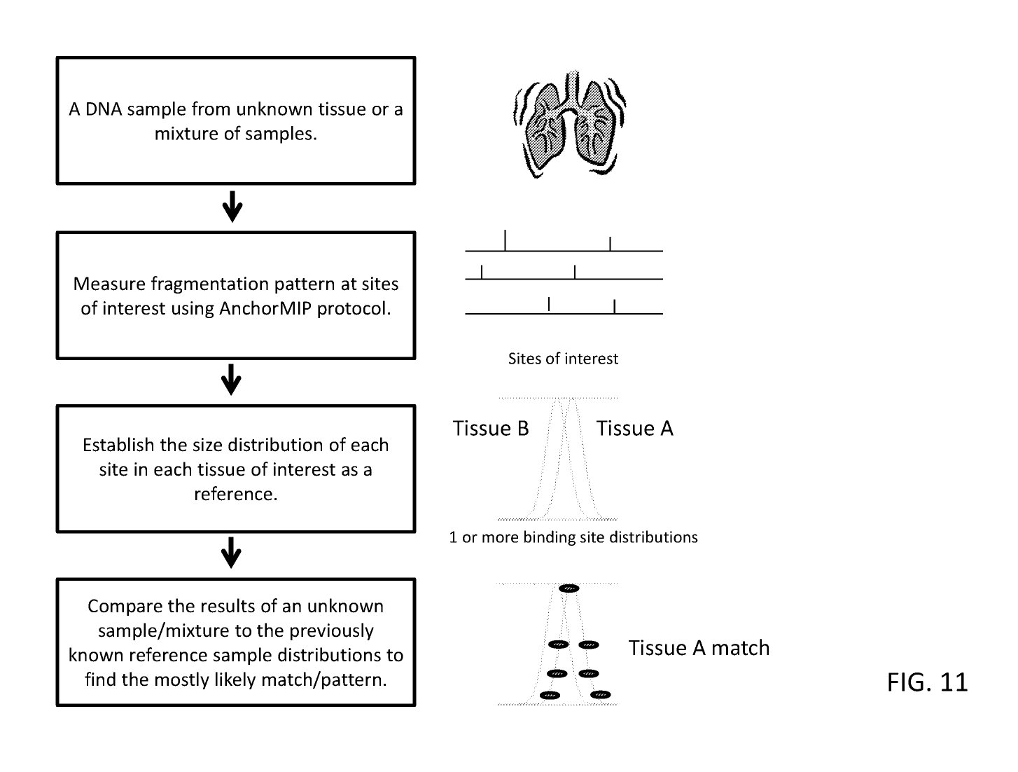

While a single CpG site in a nucleic acid molecule can be methylated or unmethylated, it is more common, and clinically useful, to determine the methylation status of a population of cells with each cell containing a unique diploid genome. In some embodiments, the compositions and methods described herein provide a methylation score for a subset of the total diploid genomes within a selected sample. Thus, the binary methylation status of a collection of single CpG sites is being summed over the population of cells to give a methylation score across many CpG sites in a sample. In some embodiments, the methylation score of a region (e.g., block, gene, chromosome, or globally) can be calculated as a median, mean or average of the individual site ratios. The methylation score can also be expressed as a ratio or a percentage. In some embodiments that employ a sequencing readout such as next generation sequencing, the methylation score is calculated from the nucleic acid sequence information contained in sequencing reads comprising CpG sites. Thus, a methylation score can be thought of as the proportion of sequence reads showing methylation at CpG sites over the total number of reads covering the CpG sites —whether methylated or not. In some embodiments, a single read can generate multiple counts if it comprises multiple CpG sites. For example in FIG. 10, the number of CpG sites covered by the assay does not necessarily dictate the methylation score. If that was the case, as illustrated in FIG. 10, the three CpG sites in both Samples 1 and 2 would be methylated and the methylation index would be 100% for both samples. In some embodiments, the methylation score can be used to determine the methylation state of a single CpG site, or a series of individual CpG sites. In some embodiments, CpG sites can be selectively filtered out of the analysis or the CpG sites can be grouped and the methylation density can be calculated.

In some embodiments, the methylation score can be expressed as the “methylation density”, which is the methylation score for the CpG sites in a defined region (e.g., a particular CpG site, CpG sites within a CpG island, or a larger region such as a block). For example, the methylation density for a 1 Mb bin in the human genome can be determined from the number of counts showing CpG methylation divided by the total number of counts covering CpG sites in the 1 Mb region. This analysis can also be performed for other bin sizes, e.g. 50 kb or 100 kb, etc.

In some embodiments, the methylation score can be expressed as the “proportion of methylated cytosines”, which includes cytosines outside of the CpG context in the region.

In some embodiments, the methylation score can be expressed as a “global methylation score” or “global methylation index”. The global methylation index refers to the methylation score for all of the CpG sites interrogated by the compositions and methods described herein, which includes CpG sites across the genome (e.g., greater than 50,000, 60,000, 70,000, 80,000, 100,000, 150,000, 200,000, 300,000, 400,000, 500,000 or more CpG sites distributed throughout the genome); thus allowing one to generate a global methylation index with a single assay. The skilled worker will appreciate that not every CpG site in a genome needs to be studied to determine the global methylation index. For example, the methylation score of a subset of CpG sites may be determined as an indication for the global methylation state of the entire genome, and given as a “global methylation index”.

In some embodiments, a methylation score is determined for a test subject, sample, tissue or portion thereof, in which case it is referred to as a “test methylation score” or “test ratio”. A test ratio can be compared to a “reference methylation score” or “reference ratio” from a corresponding known (reference) subject, sample or tissue. For example, the methylation score from a population of cells (e.g., from a tumor, or from a particular tissue type), multiple or mixed populations of cells (e.g., maternal and fetal cells), or multiple subjects (e.g., smoker vs. non-smoker) can be determined and compared to corresponding well-characterized, reference samples or subjects. As used herein, the term “maternal” in reference to a subject or a sample refers to female subject who is or who has been pregnant. It is contemplated that, in some embodiments, fetal cells and/or fetal nucleic acid may be detected in a maternal subject or sample after the end of pregnancy.

In some embodiments, the regions with methylation differences between test samples and reference samples are referred to as “differentially methylated regions” (DMRs), which are regions or blocks having different methylation scores. A differentially methylated region (e.g., block, chromosome, gene, island, etc.) is identified by a difference in the methylation score between a test and reference sample across a sufficient number of samples to be significant.

The term “site” as used herein refers to a single site, which may be a single base position or a group of correlated base positions, e.g., a CpG site; whereas a “block” or “region” refers to a portion of the genome that includes multiple sites. A block may include one or more CpG islands, genes, chromatin regions such as large organized chromatin lysine-modifications, or nuclear organization regions such as lamin-associated domains. A block may contain one or more repeat elements. The compositions and methods described herein offer improved methods for identifying large scale phenomenon of methylation dysregulation in diseases such as cancer. Rather than a specific targeting of methylation change at particular sites, the compositions and methods described are able to assay the regions of the genome believed to be pathologically important for cell differentiation and disease. Furthermore, in the case of cancer, there is evidence this epigenetic dysregulation is occurring early in cancer—even before full cancer development (see Timp et al. Genome Medicine 2014, 6:61) and is more likely to occur at CpG sites that reside in Alu repeat elements (see Luo et al. BioMed research international 2014); thereby adding to the clinical utility of the methods and compositions described herein.

The term “methylome” as used herein refers to the amount or pattern of methylation at different sites or regions within a population of cells. Thus, methylome can be thought of as the methylation score for a particular population of cells. For example, a disease state may have a methylome, such as the healthy liver methylome versus the necrotic liver. A tissue type may have a methylome, such as a liver methylome versus a blood methylome. A cellular phenotype may have a methylome, such as senescent cells versus dividing cells. The methylome may correspond to all of the genome, a subset of the genome (e.g., repeat elements in the genome), or a portion of the subset (e.g., those areas found to be associated with disease). A “fetal methylome” corresponds to a methylome of a fetus of a pregnant female. The fetal methylome can be determined using a variety of fetal tissues or sources of fetal DNA, including placental tissues and cell-free fetal DNA in maternal plasma. A “tumor methylome” corresponds to a methylome of a tumor of an organism (e.g., a human). The tumor methylome can be determined using tumor tissue or cell-free tumor DNA in maternal plasma. The fetal methylome and the tumor methylome are examples of a methylome of interest. Other examples of methylomes of interest are the methylomes of organs (e.g. methylomes of the liver, lungs, prostate, gastrointestinal tract, bladder etc.) that can contribute DNA into a bodily fluid (e.g. plasma, serum, sweat, saliva, urine, genital secretions, semen, stools fluid, diarrheal fluid, cerebrospinal fluid, secretions of the gastrointestinal tract, ascitic fluid, pleural fluid, intraocular fluid, fluid from a hydrocele (e.g. of the testis), fluid from a cyst, pancreatic secretions, intestinal secretions, sputum, tears, aspiration fluids from breast and thyroid, etc.). The organs may be transplanted organs. A methylome from plasma may be referred to a “plasma methylome”. The plasma methylome is an example of a cell-free methylome since plasma and serum include cell-free DNA (cfDNA). The plasma methylome is also an example of a mixed population methylome since it is a mixture of fetal/maternal methylome or tumor/non-tumor methylome or DNA derived from different tissues or organs.

The term “read” as used herein refers to the raw or processed output of sequencing systems, such as massively parallel sequencing. In some embodiments, the output of the methods and compositions described herein is reads. In some embodiments, these reads may need to be trimmed, filtered, and aligned resulting in raw reads, trimmed reads, aligned reads. The term “count” as used herein refers to a uniquely aligned read within a target sequence of interest. In the context of the methylation score, a count will correspond to the information retrieved from the reads (methylated or unmethylated) at the CpG sites. Therefore, if a read encompassed multiple CpG site, this read can produce multiple counts.

In certain embodiments, the methods may be used to detect copy number variations. As used herein a “copy number variation” (CNV) generally is a class or type of genetic variation or chromosomal aberration. In some contexts, copy number variations refer to changes in copy number in germline cells, while copy number alterations/aberrations (CNAs) refer changes in copy number that have arisen in somatic tissue (e.g., in tumor cells). As used herein, copy number variations include copy number alterations/aberrations. A copy number variation can be a deletion (e.g. micro-deletion), duplication (e.g., a micro-duplication), or insertion (e.g., a micro-insertion). In certain embodiments, the prefix “micro” as used herein may refer to a segment of a nucleic acid less than 5 base pairs in length. A copy number variation can include one or more deletions (e.g. micro-deletion), duplications and/or insertions (e.g., a micro-duplication, micro-insertion) of a segment of a chromosome. In certain embodiments a duplication comprises an insertion. In certain embodiments an insertion is a duplication. In certain embodiments an insertion is not a duplication. For example, a duplication of a sequence in a portion increases the counts for a portion in which the duplication is found. Often a duplication of a sequence in a portion increases the elevation or level. In certain embodiments, a duplication present in portions making up a first elevation or level increases the elevation or level relative to a second elevation or level where a duplication is absent. In certain embodiments an insertion increases the counts of a portion and a sequence representing the insertion is present (i.e., duplicated) at another location within the same portion. In certain embodiments an insertion does not significantly increase the counts of a portion or elevation or level and the sequence that is inserted is not a duplication of a sequence within the same portion. In certain embodiments an insertion is not detected or represented as a duplication and a duplicate sequence representing the insertion is not present in the same portion. In some embodiments a copy number variation is a fetal copy number variation. Often, a fetal copy number variation is a copy number variation in the genome of a fetus. In some embodiments a copy number variation is a maternal and/or fetal copy number variation. In certain embodiments a maternal and/or fetal copy number variation is a copy number variation within the genome of a pregnant female (e.g., a female subject bearing a fetus), a female subject that gave birth or a female capable of bearing a fetus. A copy number variation can be a heterozygous copy number variation where the variation (e.g., a duplication or deletion) is present on one allele of a genome. A copy number variation can be a homozygous copy number variation where the variation is present on both alleles of a genome. In some embodiments a copy number variation is a heterozygous or homozygous fetal copy number variation. In some embodiments a copy number variation is a heterozygous or homozygous maternal and/or fetal copy number variation. A copy number variation sometimes is present in a maternal genome and a fetal genome, a maternal genome and not a fetal genome, or a fetal genome and not a maternal genome.

The term “genomic instability” as used herein, refers to a high frequency of mutations within the genome of a cellular lineage. For example, there is often greater genomic instability in cancers versus adenomas. Genomic instability is often the result of DNA damage, for example as caused by faulty DNA repair genes, and can lead to aneuploidy, chromosomal translocations, chromosomal inversions, chromosome deletions, single-strand breaks in DNA, double-strand breaks in DNA, the intercalation of foreign substances into the DNA double helix, or any abnormal changes in DNA tertiary structure that can cause either the loss of DNA, or the misexpression of genes. In some embodiments, the presence or absence of copy number variations is an indication of genomic instability.

The term “aneuploidy,” as used herein, refers to a chromosomal abnormality characterized by an abnormal variation in chromosome number, e.g., a number of chromosomes that is not an exact multiple of the haploid number of chromosomes. For example, a euploid individual will have a number of chromosomes equaling 2n, where n is the number of chromosomes in the haploid individual. In humans, the haploid number is 23. Thus, a diploid individual will have 46 chromosomes. An aneuploid individual may contain an extra copy of a chromosome (trisomy of that chromosome) or lack a copy of the chromosome (monosomy of that chromosome). The abnormal variation is with respect to each individual chromosome. Thus, an individual with both a trisomy and a monosomy is aneuploid despite having 46 chromosomes. Examples of aneuploidy diseases or conditions include, but are not limited to, Down syndrome (trisomy of chromosome 21), Edwards syndrome (trisomy of chromosome 18), Patau syndrome (trisomy of chromosome 13), Turner syndrome (monosomy of the X chromosome in a female), and Klinefelter syndrome (an extra copy of the X chromosome in a male). Other, non-aneuploid chromosomal abnormalities include translocation (wherein a segment of a chromosome has been transferred to another chromosome), deletion (wherein a piece of a chromosome has been lost), and other types of chromosomal damage (e.g., Fragile X syndrome, which is caused by an X chromosome that is abnormally susceptible to damage).

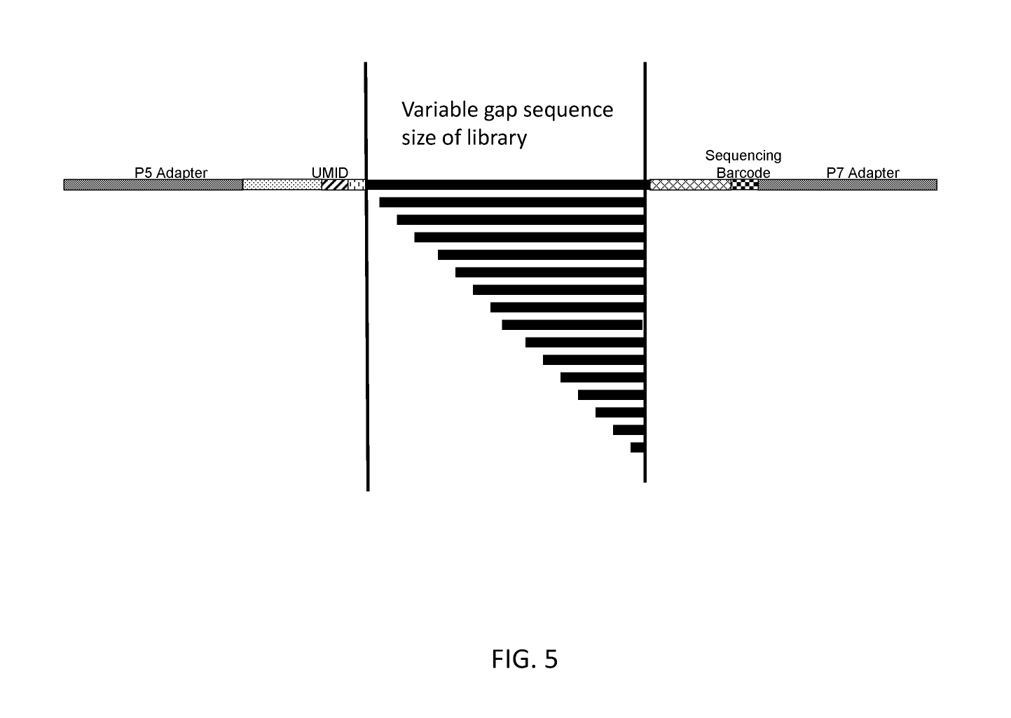

The term “nucleic acid fragment size” as used herein refers to the length of a continuous nucleic acid fragment. The length can be determined by sequencing the fragment to determine the total number of nucleotide bases present in the fragment. Other means for determining the nucleic acid fragment size, such as determining the fragments mass, can also be used. In some embodiments, the size of a population of nucleic acid fragments is determined using the compositions and methods described herein. As used herein the size of a population of nucleic acid fragments is referred to as a “size profile of nucleic acids”, “size distribution of nucleic acid fragments”, “size distribution of amplicons”, or “amplicon fragmentation pattern” wherein “amplicons” is defined herein to refer to a nucleic acid generated via capturing reactions or amplification reactions. Determining the “size profile” or “size distribution” of nucleic acid fragments accounts for both the size of the fragments as well as the relative or absolute concentration of one or more of the fragment sizes. See FIG. 5. Generally, cell-free nucleic acid fragments are created when a cell undergoes necrosis or apoptosis, and the cellular nucleic acids are digested or cleaved to create fragmented, cell-free DNA. In some embodiments, cellular or genomic DNA may be fragmented ex vivo for analysis using the compositions and methods described herein.

The terms “gene fusion event”, or simply “fusion” as used herein refer to a genomic rearrangement in which genomic sequences merge to form a new hybrid genomic sequence. As used herein, “gene fusion event” can result in a fusion gene, a chimeric gene or any other new combinations of genomic sequences. In some embodiments, the gene fusion event results in one or more fusion genes, which are generally considered a combination of whole gene sequences into a single reading frame that usually retain their original functions. In other embodiments, the gene fusion event results in one or more chimeric genes, which are generally considered a combination of portions of one or more coding sequences to produce new genes. Gene fusion events can be the result of a translocation, interstitial deletion, or chromosomal inversion.

The term “nucleosomal occupancy analysis” as used herein refers to the analysis of how DNA is organized or packaged in certain regions of the genome (e.g., the organization of chromatin around transcription factor binding sites or nucleases). This organization around specific sites differs in DNA obtained from different origins (e.g., DNA from different tissues will have different patterns of organization). Thus, DNA organization around specific sites can be used to determine the origin of the DNA. Moreover, because DNA organization can be a function of protein binding to DNA, differential protein binding between DNA molecules from differing origins of interest can result in different DNA fragment patterns, which can be used to determine the origin of those molecules. Thus, in some embodiments, the compositions and methods described herein can be used to determine the nucleosomal occupancy of DNA.

The term “mutational landscape”, as used herein, is the cumulative frequency of a collection of mutations that generally span the genome. The types of mutations that make up a mutational landscape, include but are not limited to, single nucleotide variations, deletions, insertions, translocations, fusions, and repeat expansions, and the type of mutations also inform a given mutational landscape or pattern. Examples of specific mutational landscapes associated with diseases or conditions include an increased frequency of C>A transversions associated with cigarette smoke exposure (Ding, L. et al. Somatic mutations affect key pathways in lung adenocarcinoma. Nature 455, 1069-1075 (2008)), and an increased frequency of C>T transitions and C>G transversions associated with 12 cancer types (Kandoth, Cyriac, et al. “Mutational landscape and significance across 12 major cancer types.” Nature 502.7471 (2013): 333-339).

The term “tissue-of-origin” as used herein refers to the tissue source of nucleic acids in a sample, where “tissue” is used to describe a group or population of cells of a same type. Some tissue may have multiple cell types, for example hepatocytes, alveolar cells or blood cells, while other tissue may originate from different organisms, for example, mother and fetus, or from healthy vs. disease tissue. Likewise, “reference tissues” may be used to determine methylation-specific or size-specific tissue patterns or levels. For example, reference tissues from multiple subjects may be used to determine the tissue components of a test sample. In some embodiments, the DNA molecules of differing origin are DNA molecules of maternal origin and DNA molecules of fetal origin. In some embodiments, the DNA molecules of differing origin are DNA molecules of a first tissue origin and DNA molecules of a second tissue origin or of leukocyte origin. In some embodiments, determining the tissue-of-origin may be indicative of the presence of disease (e.g., cancer), or may be used to determine the relative or absolute amount of DNA from a particular tissue (e.g., fetal cfDNA in a maternal sample). Thus, the compositions and methods described herein can be used to differentiate and identify the tissue source of DNA. Given an unknown source of tissue, DNA can be extracted using standard methods, the compositions and methods described herein can be used to generate tissue-specific data, and the data can be fit into the most likely tissue reference bin as described further in the Examples.

The terms “subject” and “patient”, as used herein, refer to any animal, such as a dog, a cat, a bird, livestock, and particularly a mammal, and preferably a human. The term “test subject” and “test patients” refer to any subject or patient with an unknown genetic or methylation status. In some embodiments, the genetic or methylation status of a test subject is determined using the compositions and methods described herein. In some embodiments, the genetic or methylation status of a test subject is compared to a reference subject or reference patient. The term “reference subject” and “reference patients” refer to any subject or patient that exhibit known genotypes (e.g., known euploidy or aneuploidy), phenotypes, ages, or is otherwise well characterized. Reference subjects may also be known to have a disease or condition, or known to have a particular state of a disease or condition, or known to have a predisposition to a disease or condition, or known to have been exposed to drugs, toxins, a particular diet, or an agent or conditions suspected of causing methylation changes. The genetic or methylation status of a test subject can be expressed as a ratio, score or index, wherein one or more of the multimodal metrics (e.g., nucleic acid size profile, methylation status, genomic instability status, mutational landscape status, genomic rearrangement status) determined using the compositions and methods described herein is compared to a reference. The skilled worker will appreciate that the subject can be any human. In certain embodiments, the subject is a pregnant female. In these embodiments, the blood sample may be a maternal serum plasma or serum sample. In certain embodiments, the subject is an organ transplant recipient, and the subject's methylation state may be indicative of organ rejection. In certain embodiments, the methylation state of a population of target sequences of interest (e.g., of fetal, tumor, or disease origin) may be determined among a background of target sequences of interest (e.g., maternal, non-tumor, or disease-free origin). The background target sequences of interest may serve as a reference, wherein differences from the reference are indicative of a disease or condition, or to identify a nucleic acid species.

The terms “polynucleotide”, “nucleic acid” and “nucleic acid molecules”, as used herein, are used interchangeably and refer to DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNAs), microRNA (miRNAs), small nucleolar RNA (snoRNAs), ribosomal RNA (rRNA), transfer RNA (tRNA), small interfering RNA (siRNA), heterogeneous nuclear RNAs (hnRNA), or small hairpin RNA (shRNA). In certain embodiments, the methods can be performed on a nucleic acid sample such as DNA or RNA, e.g., genomic DNA. In some embodiments the nucleic acid molecule may be cell-free DNA (cfDNA). Cell-free DNA is thought to result from cellular necrosis or apoptosis, wherein genomic cellular DNA is digested and becomes fragmented, extracellular DNA. Cell-free DNA of apoptotic origin may be from a non-host (e.g., transplanted organ or tissue), fetus (e.g., from the placenta resulting in cell-free fetal DNA), or a diseased tissue (e.g., from a tumor resulting in circulating tumor DNA). Cell-free DNA can be detected in a range of samples including, but not limited to, blood, plasma and urine. In some embodiments, the nucleic acid molecules are associated with exosomes, which are microvesicles released from a variety of different cells, including cancer cells. In some embodiments, the compositions and methods described herein may be able to differentiate cfDNA of necrotic and apoptotic origin based on its methylation status or size profile. A nucleic acid sample may be isolated in any manner known to a person of ordinary skill in the art (e.g., by centrifugation).

The term “sample”, as used herein, refers to a sample typically derived from a biological fluid, cell, tissue, organ, or organism, comprising a nucleic acid or a mixture of nucleic acids comprising at least one nucleic acid sequence that is to be screened for, e.g., cancer or aneuploidy. In some embodiments, a sample is a blood sample such as a whole blood sample, a serum sample, or a plasma sample. In some embodiments the sample comprises at least one nucleic acid sequence whose genome is suspected of having undergone variation. Such samples include, but are not limited to sputum/oral fluid, amniotic fluid, blood, a blood fraction, or fine needle biopsy samples (e.g., surgical biopsy, core needle biopsy, fine needle biopsy, etc.) urine, stool, peritoneal fluid, pleural fluid, cerebro-spinal fluid, gastrointestinal fluid, cell lines, tissue embedded in paraffin, fresh frozen tissue, and the like. Although the sample is often taken from a human subject (e.g., patient), the assays can be used to detect a disease or condition, or detect the state of a disease or condition, or determine whether a subject has a predisposition to a disease or condition, in samples from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, bisulfate conversion, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, preferably at a concentration proportional to that in an untreated test sample (e.g., namely, a sample that is not subjected to any such pretreatment method(s)). Depending on the type of sample used, additional processing and/or purification steps may be performed to obtain nucleic acid fragments of a desired purity or size, using processing methods including but not limited to sonication, nebulization, gel purification, PCR purification systems, nuclease cleavage, size-specific capture or exclusion, targeted capture or a combination of these methods. Optionally, cell-free DNA may be isolated from the sample prior to further analysis. In some embodiments, the sample is from the subject whose disease or condition is to be determined by the systems and methods of the invention, also referred as “a test sample.”

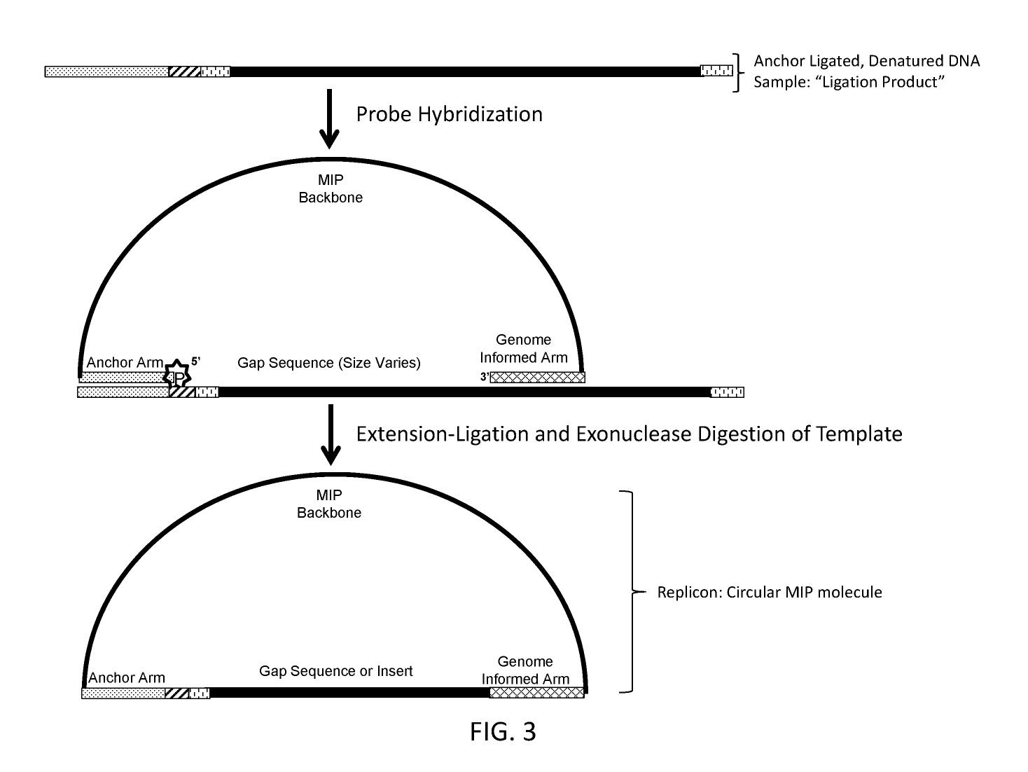

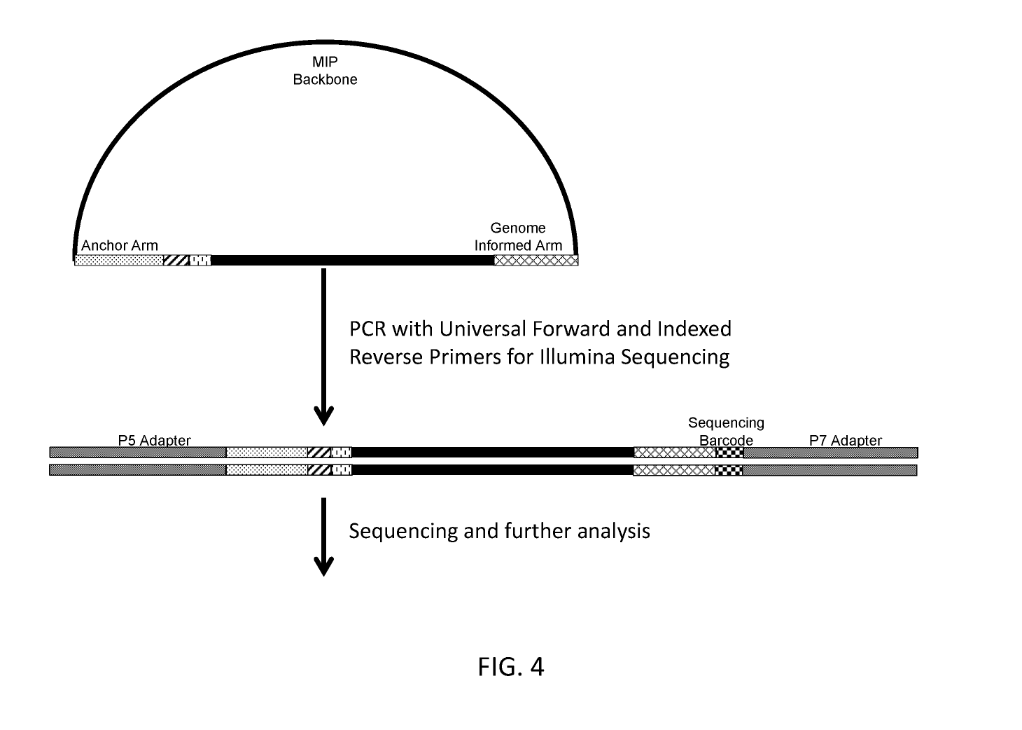

The term “MIP,” as used herein, refers to a molecular inversion probe (also known as a circular capture probe). As used herein, the terms “primer”, “probe”, or “capture probe” also may refer to a MIP in the context of their ability to selectively bind to nucleic acid molecules. Molecular inversion probes are nucleic acid molecules that contain two targeting polynucleotide arms (e.g., an anchor arm and a genome-informed arm), one or more unique molecular tags (also known as unique molecular identifiers (UMID's)), and a polynucleotide linker (e.g., a universal backbone linker). A polynucleotide linker can range from 10, 20, 30, 40, 50, 60, 70, 80, 90, 100, 125, 150, 175, 200, 225, 250, 275, 300, 400, 500, 1000, 1500, 2000 or more bases. See, for example, FIG. 3 and FIG. 4. In some embodiments, a MIP may comprise more than one unique molecular tags, such as, two unique molecular tags, three unique molecular tags, or more. In some embodiments, the polynucleotide arms in each MIP are located at the 5′ and 3′ ends of the MIP, while the unique molecular tag(s) and the polynucleotide linker are located in the middle. For example, in some embodiments, the MIPs comprise in sequence the following components: anchor arm—first unique molecular tag—polynucleotide linker—second unique molecular tag—genome-informed arm. In some embodiments, the polynucleotide linker (or the backbone linker) in the MIPs are universal in all the MIPs used in a method of the invention. In some embodiments, the MIPs may not comprise any unique molecular tags.

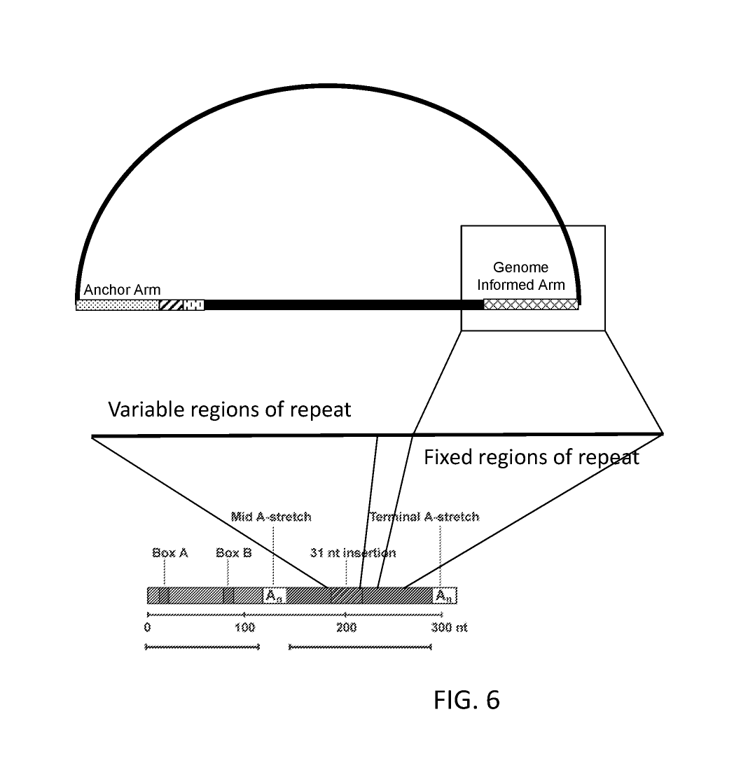

In the MIPs, the polynucleotide arms, which consist of an “anchor arm” and a “genome-informed arm” are designed to hybridize upstream and downstream of target sequences (or sites) in a genomic nucleic acid sample. More specifically, the “anchor arms” are designed to bind to “anchor sequences” and the “genome-informed arms” are designed to bind to a repeat sequence found across the genome, wherein the anchor sequences and repeat sequences flank a target sequence. The target sequences comprise a “gap sequence” or a “unique gap sequence” that is used to uniquely align the target sequence back to the genome. In some embodiments, the gap sequences are 1, 2, 3, 4, 5, 6, 7, 8, 9, 10, 11, 12, 13, 14, 15, 16, 17, 18, 19, 20, 25, 30, 40, 50, 60, 70, 80, 90, 100, 100, 125, 150, 175, 200, 225, 250, 275, 300, 400, 500, 1000, 1500, 2000 bases or greater in length. When interrogating cell-free DNA, the gap sequences are generally less than 150 or 200 bases in length. A MIP may comprise an anchor arm that is substantially complementary to an anchor sequence that is introduced to the target sequences via a ligation reaction. A MIP may comprise a genome-informed arm that is substantially complementary to a plurality of repeat sequences in a DNA sample. In some embodiments, the genome-informed arm binds to the fixed regions of repeat elements such as Alu repeat elements. See FIG. 6. In some embodiments, a MIP can hybridize to tens, hundreds, thousands, hundreds of thousands, or millions of target sequences of interest in a DNA sample (e.g., a sample comprising a human genome). In some embodiments, a MIP targets, for example, greater than 1,000, greater than 10,000, greater than 20,000, greater than 30,000, greater than 40,000, greater than 50,000, greater than 60,000, greater than 70,000, greater than 80,000, greater than 90,000, greater than 100,000, greater than 200,000, greater than 300,000, greater than 400,000, greater than 500,000, greater than 600,000, greater than 700,000, greater than 800,000, greater than 900,000, and/or greater than 1,000,000 target sequences of interest. In some embodiments, “substantially complementary” refers to 0 mismatches in both arms, or at most 1 mismatch in only one arm (e.g., when the targeting polynucleotide arms hybridize to the first and second regions in the nucleic acid that, respectively, flank a site of interest). In some embodiments, “substantially complementary” refers to at most a small number of mismatches in both arms, such as 1, 2, 3, 3, 5, 6, 7, or 8.

The terms “target sequence”, “sequence of interest”, and “target sequence of interest” are used interchangeably to refer to the sequence bound or captured by the primes or probes of the invention. These target sequences of interest may include a repeat sequence to which the genome-informed arm hybridizes. In certain embodiments, the repeat sequences have 0, 1, 2, 3, 4, or more mismatches in hybridizing with the genome-informed arm. In specific embodiments, the repeat sequences have 0 or 1 mismatches in hybridizing with the genome-informed arms. In some embodiments, a capture probe binds to Alu repeats. In some embodiments, a capture probe does not bind long interspersed nucleotide elements (LINE) in the genome.

The term “random priming” as used herein refers to a process whereby anchor sequences (or any sequence) can be added to single-stranded nucleic acids, whether bisulfite-treated or not, using random primers that include an anchor sequence. Random priming was first described by Feinberg and Vogelstein (See “A technique for radiolabeling DNA restriction endonuclease fragments to high specific activity”, Ann. Biochem. 132, 6-13 (1983)).

In some embodiments, the unique molecular tags are short nucleotide sequences that are randomly generated. In certain embodiments, the unique molecular tags are not designed to hybridize to any sequence or site located on a genomic nucleic acid fragment or in a genomic nucleic acid sample. In certain embodiments, the unique molecular tag is any tag with a suitable detectable label that can be incorporated into or attached to a nucleic acid (e.g., a polynucleotide) that allows detection and/or identification of nucleic acids that comprise or attach to the tag. In certain embodiments unique molecular tags of sufficient length are introduced at concentrations to ensure each MIP comprises a unique combination of molecular tags, thereby making each capture event distinct. By tracking individual capture events, one is able to identify duplicates and reduce capture bias. Although the invention described herein already allows for nearly uniform capture efficiencies since the same capture probe is used to interrogate many sites across the genome, the ability to account for capture bias (i.e., normalizing for differences in capture efficiency), further improves the quantitative aspects of the assay, for example, when doing CNV analysis or determining the size distribution of nucleic acid fragments. In some embodiments the tag is incorporated into or attached to a nucleic acid during a sequencing method (e.g., by a polymerase). Non-limiting examples of tags include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments the tag (e.g., a nucleic acid index or barcode) is a unique, known and/or identifiable sequence of nucleotides or nucleotide analogues. In some embodiments the tags or UMID's help reduce or remove amplification errors and sequencing errors by allowing for the identification of unique molecules during bioinformatics analysis. In some embodiments tags are four, five, or six or more contiguous nucleotides. Unique molecular identifiers comprising oligonucleotides are described in U.S. patent application Ser. No. 11/186,636, which published as US20070020640A1, and the use of oligonucleotide unique molecular identifiers with MIPs is described in U.S. patent application Ser. No. 12/027,039, which published as US20080269068A1. A multitude of fluorophores are available with a variety of different excitation and emission spectra. Any suitable type and/or number of fluorophores can be used as a tag. In some embodiments 1 or more, 2 or more, 3 or more, 4 or more, 5 or more, 6 or more, 7 or more, 8 or more, 9 or more, 10 or more, 20 or more, 30 or more, 50 or more, 100 or more, 500 or more, 1000 or more, 10,000 or more, 100,000 or more, 10\hat{ }7 or more, 10\hat{ }8 or more, 10\hat{ }9 or more, 10\hat{ }10 or more, 10\hat{ }11 or more, 10\hat{ }12 or more different tags are utilized in a method described herein (e.g., a nucleic acid detection and/or sequencing method). In some embodiments, one or two types of tags (e.g., fluorescent labels) are linked to each nucleic acid in a library. In some embodiments, chromosome-specific tags are used to make chromosomal counting faster or easier. Detection and/or quantification of a tag can be performed by a suitable method, machine or apparatus, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof. In particular embodiments, the tag is suitable for use with microarray analysis.

In certain alternative embodiments, the MIPs of the invention may not comprise any unique molecular tags. It is possible to determine the methylation status, copy number variation, mutational landscape, etc. using MIPs that do not contain unique molecular tags, according to the methods of the disclosure.

In certain alternative embodiments, a single oligonucleotide MIP of the invention ranging in size between 70-110 bases has polynucleotide arms, which consist of Clip arms designed to capture target sequences (or sites) in a genomic nucleic acid sample. More specifically, the “Clip binding arms” are designed to bind to both ends of a fragmented target sequence (e.g., cell-free nucleic acid). A single capture probe is created that binds to the Clip sequences added to the 5′ and 3′ ends of a DNA fragment. Thus, a ClipMIP is an alternative form of a FireMIP which has anchor arms on both ends of the target sequence. In some embodiments, the Clip sequences are designed to hybridize and ligate at sites where genomic DNA is commonly cleaved, for example, following cell apoptosis. In another embodiment, cleavage sites are introduced in genomic DNA during a first step that introduces cleavage sites, for example via a restriction endonuclease. In some embodiments the portion of the Clip sequences that binds to the ClipMIP Arms were randomly-generated, exogenous sequences that do not appear in the genome. The Clip sequences can be designed to hybridize to a range of targets depending on the intended use. For example, the Clip sequences can target repeat regions (such as but not limited to Alu repeats), specific loci, restriction sites, transcription factor binding sites, or randomly fragmented ends (e.g., by using 4-6 degenerate bases on the Clip sequence).

The MIPs are introduced to nucleic acids (e.g., nucleic acid fragments) to perform capture of target sequences or sites located on a nucleic acid sample (e.g., a genomic DNA). In some embodiments, for example, if genomic DNA is present in a sample, fragmenting may aid in capture of target nucleic acid by molecular inversion probes. As described in greater detail herein, after capture of the target sequence (e.g., locus) of interest, the captured target may further be subjected to an enzymatic gap-filling and ligation step, such that a copy of the target sequence is incorporated into a circle, which is herein referred to as a replicon. Capture efficiency of the MIP to the target sequence on the nucleic acid fragment can be improved by lengthening the hybridization and gap-filing incubation periods. (See, e.g., Turner E H, et al., Nat Methods. 2009 Apr. 6:1-2.).

MIP technology may be used to detect or amplify particular nucleic acid sequences in complex mixtures. One of the advantages of using the MIP technology is in its capacity for a high degree of multiplexing, which allows thousands of target sequences to be captured in a single reaction containing thousands of MIPs. Various aspects of MIP technology are described in, for example, Hardenbol et al., “Multiplexed genotyping with sequence-tagged molecular inversion probes,” Nature Biotechnology, 21(6): 673-678 (2003); Hardenbol et al., “Highly multiplexed molecular inversion probe genotyping: Over 10,000 targeted SNPs genotyped in a single tube assay,” Genome Research, 15: 269-275 (2005); Burmester et al., “DMET microarray technology for pharmacogenomics-based personalized medicine,” Methods in Molecular Biology, 632: 99-124 (2010); Sissung et al., “Clinical pharmacology and pharmacogenetics in a genomics era: the DMET platform,” Pharmacogenomics, 11(1): 89-103 (2010); Deeken, “The Affymetrix DMET platform and pharmacogenetics in drug development,” Current Opinion in Molecular Therapeutics, 11(3): 260-268 (2009); Wang et al., “High quality copy number and genotype data from FFPE samples using Molecular Inversion Probe (MIP) microarrays,” BMC Medical Genomics, 2:8 (2009); Wang et al., “Analysis of molecular inversion probe performance for allele copy number determination,” Genome Biology, 8(11): R246 (2007); Ji et al., “Molecular inversion probe analysis of gene copy alternations reveals distinct categories of colorectal carcinoma,” Cancer Research, 66(16): 7910-7919 (2006); and Wang et al., “Allele quantification using molecular inversion probes (MIP),” Nucleic Acids Research, 33(21): e183 (2005), each of which is hereby incorporated by reference in its entirety for all purposes. See also in U.S. Pat. Nos. 6,858,412; 5,817,921; 6,558,928; 7,320,860; 7,351,528; 5,866,337; 6,027,889 and 6,852,487, each of which is hereby incorporated by reference in its entirety for all purposes.

MIP technology has previously been successfully applied to other areas of research, including the novel identification and subclassification of biomarkers in cancers. See, e.g., Brewster et al., “Copy number imbalances between screen- and symptom-detected breast cancers and impact on disease-free survival,” Cancer Prevention Research, 4(10): 1609-1616 (2011); Geiersbach et al., “Unknown partner for USP6 and unusual SS18 rearrangement detected by fluorescence in situ hybridization in a solid aneurysmal bone cyst,” Cancer Genetics, 204(4): 195-202 (2011); Schiffman et al., “Oncogenic BRAF mutation with CDKN2A inactivation is characteristic of a subset of pediatric malignant astrocytomas,” Cancer Research, 70(2): 512-519 (2010); Schiffman et al., “Molecular inversion probes reveal patterns of 9p21 deletion and copy number aberrations in childhood leukemia,” Cancer Genetics and Cytogenetics, 193(1): 9-18 (2009); Press et al., “Ovarian carcinomas with genetic and epigenetic BRCA1 loss have distinct molecular abnormalities,” BMC Cancer, 8:17 (2008); and Deeken et al., “A pharmacogenetic study of docetaxel and thalidomide in patients with castration-resistant prostate cancer using the DMET genotyping platform,” Pharmacogenomics, 10(3): 191-199 (2009), each of which is hereby incorporated by reference in its entirety for all purposes.

MIP technology has also been applied to the identification of new drug-related biomarkers. See, e.g., Caldwell et al., “CYP4F2 genetic variant alters required warfarin dose,” Blood, 111(8): 4106-4112 (2008); and McDonald et al., “CYP4F2 Is a Vitamin K1 Oxidase: An Explanation for Altered Warfarin Dose in Carriers of the V433M Variant,” Molecular Pharmacology, 75: 1337-1346 (2009), each of which is hereby incorporated by reference in its entirety for all purposes. Other MIP applications include drug development and safety research. See, e.g., Mega et al., “Cytochrome P-450 Polymorphisms and Response to Clopidogrel,” New England Journal of Medicine, 360(4): 354-362 (2009); Dumaual et al., “Comprehensive assessment of metabolic enzyme and transporter genes using the Affymetrix Targeted Genotyping System,” Pharmacogenomics, 8(3): 293-305 (2007); and Daly et al., “Multiplex assay for comprehensive genotyping of genes involved in drug metabolism, excretion, and transport,” Clinical Chemistry, 53(7): 1222-1230 (2007), each of which is hereby incorporated by reference in its entirety for all purposes. Further applications of MIP technology include genotype and phenotype databasing. See, e.g., Man et al., “Genetic Variation in Metabolizing Enzyme and Transporter Genes: Comprehensive Assessment in 3 Major East Asian Subpopulations With Comparison to Caucasians and Africans,” Journal of Clinical Pharmacology, 50(8): 929-940 (2010), which is hereby incorporated by reference in its entirety for all purposes.

The term “capture” or “capturing”, as used herein, refers to the binding or hybridization reaction between a primer or probe (e.g., molecular inversion probe) and the corresponding targeting site.

The term “sensitivity”, as used herein, refers to a statistical measure of performance of an assay (e.g., method, test), calculated by dividing the number of true positives by the sum of the true positives and the false negatives.

The term “specificity”, as used herein, refers to a statistical measure of performance of an assay (e.g., method, test), calculated by dividing the number of true negatives by the sum of true negatives and false positives.

The term “A tailing” as used herein refers to a step in the process of adding anchor sequences to fragmented, double-stranded target nucleic acid via ligation. In some methods of this disclosure, target nucleic acid undergoes end-repair, clean-up and A-tailing. A-tailing refers to the enzymatic addition of non-templated nucleotides (in this case adenosines) to the 3′ end of a blunt, double-stranded DNA molecule.

The term “amplicon”, as used herein, refers to a nucleic acid generated via capturing reactions or amplification reactions. In some embodiments, the amplicon is a single-stranded nucleic acid molecule. In some embodiments, the amplicon is a single-stranded circular nucleic acid molecule. In some embodiments, the amplicon is a double-stranded nucleic acid molecule. For example, a MIP captures or hybridizes to a target sequence or site. After the capturing reaction or hybridization, a ligation/extension mixture is introduced to extend and ligate the gap region between the two targeting polynucleotide arms to form a single-stranded circular nucleotide molecule, i.e., a MIP replicon. The gap-filled sequence in the replicon can be thought of as an “insert” or “insert sequence”. The MIP replicon may be amplified through a polymerase chain reaction (PCR) to produce a plurality of MIP amplicons, which are double-stranded nucleotide molecules. MIP replicons and amplicons can be produced from a first plurality of target sequences of interest (e.g., sequences containing known or suspected CpG sites) and a second plurality of target sequences of interest (e.g., target sequences distributed throughout the genome).

The term “sequencing”, as used herein, is used in a broad sense and may refer to any technique known in the art that allows the order of at least some consecutive nucleotides in at least part of a nucleic acid to be identified, including without limitation at least part of an extension product or a sequence insert. Sequencing also may refer to a technique that allows the detection of differences between nucleotide bases in a nucleic acid sequence. Exemplary sequencing techniques include targeted sequencing, single molecule real-time sequencing, electron microscopy-based sequencing, transistor-mediated sequencing, direct sequencing, random shotgun sequencing, Sanger dideoxy termination sequencing, targeted sequencing, exon sequencing, whole-genome sequencing, sequencing by hybridization (e.g., in an array such as a microarray), pyrosequencing, capillary electrophoresis, gel electrophoresis, duplex sequencing, cycle sequencing, single-base extension sequencing, solid-phase sequencing, high-throughput sequencing, massively parallel shotgun sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, ion semiconductor sequencing, nanoball sequencing, nanopore sequencing, 454 sequencing, Solexa Genome Analyzer sequencing, miSeq (Illumina), HiSeq 2000 (Illumina), HiSeq 2500 (Illumina), Illumina Genome Analyzer (Illumina), Ion Torrent PGM™ (Life Technologies), MinION™ (Oxford Nanopore Technologies), real-time SMRT™ technology (Pacific Biosciences), the Probe-Anchor Ligation (cPAL™) (Complete Genomics/BGI), SOLID® sequencing, MS-PET sequencing, mass spectrometry, and a combination thereof. In some embodiments, sequencing comprises detecting the sequencing product using an instrument, for example but not limited to an ABI PRISM® 377 DNA Sequencer, an ABI PRISM® 310, 3100, 3100-Avant, 3730, or 373OxI Genetic Analyzer, an ABI PRISM® 3700 DNA Analyzer, or an Applied Biosystems SOLiD™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel sequencing (MPS).

The methods and compositions described herein may alternatively employ microarray technology to quantify MIPs products. “Microarray” or “array” refers to a solid phase support having a surface, preferably but not exclusively a planar or substantially planar surface, which carries an array of sites containing nucleic acids such that each site of the array comprises substantially identical or identical copies of oligonucleotides or polynucleotides and is spatially defined and not overlapping with other member sites of the array; that is, the sites are spatially discrete. The array or microarray can also comprise a non-planar interrogatable structure with a surface such as a bead or a well. The oligonucleotides or polynucleotides of the array may be covalently bound to the solid support, or may be non-covalently bound. Conventional microarray technology is reviewed in, e.g., Schena, Ed., Microarrays: A Practical Approach, IRL Press, Oxford (2000). “Array analysis”, “analysis by array” or “analysis by microarray” refers to analysis, such as, e.g., sequence analysis, of one or more biological molecules using a microarray. In some embodiments each sample is hybridized individually to a single microarray. In other embodiments, processing through-put can be enhanced by physically connecting multiple microarrays onto a single multi-microarray plate for convenient high-throughput handling. In certain embodiments, custom DNA microarrays, for example from Affymetrix Inc. (Santa Clara, Calif., USA), can be manufactured to specifically quantify products of the MIPs assay.

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof.

This invention will be better understood from the Experimental Details which follow. However, one skilled in the art will readily appreciate that the specific methods and results discussed are merely illustrative of the invention as described more fully in the embodiments that follow thereafter.

### Methods for Analyzing Nucleic Acids

Existing methods for determining nucleic acid sequences generally employ different techniques that are either incomplete, cumbersome or relatively expensive. For example, whole genome sequencing techniques inherently require a relatively expensive library preparation process and large numbers of reads to achieve sufficient coverage. On the other hand, targeted sequencing approaches require thousands of multiplexed PCR primers to achieve partial coverage across the genome; while shotgun sequencing approaches are random and generate incomplete sequence information that require time-consuming bioinformatics to process sometimes redundant, non-informative sequence information. Further, the above techniques also require paired-end sequencing to get nucleic acid size information. This adds complexity, time and cost. The compositions and methods described herein offer a lower cost, simplified assay with a single capture probe that generates a clinically-useful, multimodal genetic and epigenetic landscape, and works on a range of nucleic acid analytes, including circulating, cell-free DNA. The easy workflow and low cost is driven by, among other things, high throughput library preparation methods for massively parallel sequencing and relatively low read depth requirements vis-à-vis other methods that offer similar nucleic acid information.

The use of repeat sequences in the optimized capture method allows dense tiling of a target area with little or no interference of similar sequences in the production of barcoded targets for single molecule kinetics during library preparation. The use of fixed anchor sequences allows for size information to be gleaned from the sequencing data, which can be used to determine clinically-useful information like tissue-of-origin for a population or subset population of nucleic acids. In some embodiments, the method has economic benefits over previous methods. In particular, these methods provide savings from the use of a small number of capture reagents (primers or probes) that still are capable of surveying genome-wide indices. Moreover, the capture reagents can provide information not only about methylation status, but also more generally about the sequences of the target sites. This information can be used to determine, for example, copy number variation, nucleosomal occupancy or mutational landscape. This information also can be used to detect chromosomal abnormalities, e.g., aneuploidies such as trisomy, or tissue-specific methylation scores and patterns along with disease or condition-specific mutational landscapes or patterns, e.g., the presence of tissue-specific circulating tumor DNA (ctDNA) in blood. In some embodiments, the compositions and methods described herein are useful for identifying subsets of the target nucleic acids indicative of a disease or condition, or useful for differentiating or enriching species of nucleic acids. The subsets of target nucleic acids may be regions found to be differentially-methylated in diseased subjects or between tissue types, susceptible to genomic instability, or containing high frequency mutations or genomic rearrangements.

In some embodiments, the methods also provide a rapid analysis with a low read count in an assay that is easily multiplexed. For example, multiple layers of unique molecular tags and/or barcodes can be used within the methods to identify specific primer species as well as to deconvolute multiplex data to trace signals back to individual samples. For example, a first population of MIPs can be used to obtain methylation status (and optionally, sequence information), while a second population of MIPs provides sequence information. Moreover, the methods can be used in ultra-low coverage applications such as detecting trisomies in a 100% fetal sample, such as a product of conception, or a non-fetal diagnostic sample. A sample can be mixed (e.g., fetal vs. maternal or diseased vs. non-diseased) or not mixed (e.g., an individual suspected of having a disease or condition), in which case the “coverage” or read depth can be lower because the signal will be strong. In some embodiments, the methods also are fast as compared to whole genome sequencing, whole exome sequencing, and targeted sequencing. The methods described herein also offer the advantage of requiring relatively small amounts of input DNA as compared to whole genome bisulfite sequencing, which suffers from input DNA loss during the harsh bisulfite conversion process, whereas the methods described herein allow for the capture of bisulfite converted DNA after the conversion step thereby preserving input DNA and reducing bias. More specifically, most library prep kits require double-stranded input DNA for an adapter ligation step. Since bisulfite conversion denatures the DNA, the bisulfite conversion step needs to be performed after the adapter ligation step, but before PCR. The harsh bisulfite conversion can compromise some of the ligated molecules, and thereby make them unusable. Also, using conventional methods, the ligation adapters need to be methylated, otherwise the cytosines will be converted, which adds additional cost.

In some embodiments, the methods are related to the field of genetic analysis. In general, these methods can be used as a rapid and economical means to detect and quantify one or more of genomic instability, CNV status, mutational landscape, tissue-of-origin, or methylation status. Because the methylation status can be determined by sequencing, the sequence information obtained by the methods described herein allows for detection of mutations as well as detection of deletions and duplications of genetic features in a range extending from complete chromosomes and arms of chromosomes to microscopic deletions and duplications, submicroscopic deletions and deletions, and even single nucleotide features including single nucleotide polymorphisms, deletions, and insertions. In certain embodiments, these methods can be used to detect sub-chromosomal genetic lesions, e.g., microdeletions. Moreover, the methods can be used to determine mutations or other sequence elements that correlate to a disease or condition (e.g., by detecting a SNP or SNPs). Because the methods provide different types of information in a single assay, they are simpler, more efficient, and less expensive than current methods. In certain embodiments, the methods also provide a maximum likelihood estimate (k) which will allow for increased accuracy and an estimation of the probe capture efficiency and reduces need for extraneous sequencing during copy number variation (CNV) detection. This may result in a low coefficient of variation (CV) due to probe uniformity because a small number of probes (e.g., one, two or more) is used. These capture probes allow the combination of additional probes with no interference or cross assay reactions. Combining the information from several probes and their unique reads greatly reduces error in the system. Indeed, targeted probe addition can greatly enhance assay utility while reducing cost.

The compositions and methods described herein can be used to assemble methylomes through the sequence analysis of plasma DNA. The ability to determine the placental or fetal methylome from maternal plasma provides a noninvasive method to determine, detect and monitor the aberrant methylation profiles associated with pregnancy-related conditions such as preeclampsia, intrauterine growth restriction, preterm labor and others. In addition to the direct applications on the investigation of pregnancy-associated conditions, the approach can also be applied to other areas of medicine where plasma DNA analysis is of interest. For example, the methylomes of cancers can be determined from plasma DNA of cancer patients. Cancer methylome analysis from plasma, as described herein, is potentially a synergistic technology to cancer genomic analysis from plasma (e.g., the detection of well-known cancer-associated somatic mutations, or of genome wide mutational landscape).

For earlier cancer detection, the determination of one or more of genomic instability, CNV status, mutational landscape, tissue-of-origin, or methylation status can be used to screen for cancer. For example, when the mutational landscape of a plasma sample shows aberrant levels (test ratio) compared with healthy controls (reference ratio), cancer may be suspected, and the compositions and methods described herein may be able to further deduce the tissue-of-origin of the aberrant levels. Using the compositions and methods described herein, further confirmation and assessment of the type of cancer or tissue-of-origin of the cancer may be performed. The compositions and methods described herein also allow for the detection of tumor-associated copy number aberrations (often associated with genomic instability), chromosomal translocations and single nucleotide variants across the genome (mutational landscape). In some embodiments, radiological and imaging investigations (e.g. computed tomography, magnetic resonance imaging, positron emission tomography) or endoscopy (e.g. upper gastrointestinal endoscopy or colonoscopy) can be used to further investigate individuals who are suspected of having cancer based on the genetic and epigenetic profiles generated using the compositions and methods described herein.

For cancer screening or detection, the determination of one or more of genomic instability, CNV status, mutational landscape, tissue-of-origin, or methylation status of a plasma (or other biologic) sample can be used in conjunction with other modalities for cancer screening or detection such as prostate specific antigen measurement (e.g. for prostate cancer), carcinoembryonic antigen (e.g. for colorectal carcinoma, gastric carcinoma, pancreatic carcinoma, lung carcinoma, breast carcinoma, medullary thyroid carcinoma), alpha fetoprotein (e.g. for liver cancer or germ cell tumors), CA125 (e.g. for ovarian and breast cancer) and CA19-9 (e.g. for pancreatic carcinoma).

Additionally, other tissues may be sequenced to generate genetic and epigenetic profiles. For example, liver tissue can be analyzed to determine a methylation or size pattern specific to the liver, which may be used to identify liver pathologies. Other tissues which can also be analyzed include brain cells, bones, the lungs, the heart, the muscles and the kidneys, etc. The methylation or size profiles of various tissues may change from time to time, e.g. as a result of development, aging, disease processes (e.g. inflammation or cirrhosis or autoimmune processes (such as in systemic lupus erythematosus)) or treatment (e.g. treatment with demethylating agents such as 5-azacytidine and 5-azadeoxycytidine). The dynamic nature of DNA methylation makes such analysis potentially valuable for monitoring of physiological and pathological processes. For example, if one detects a change in the plasma methylome of an individual compared to a baseline value obtained when they were healthy, one could then detect disease processes in organs that contribute plasma DNA.

The methods provided by some embodiments have particular advantages as compared to targeted sequencing. In certain embodiments, the methods described herein use a simultaneous recognition of two sequence elements at the point of capture, and the two arms are limited by proximity. By contrast, a typical targeted sequencing method will allow a polymerase to initiate at a single site. The run on-product created by typical sequencing produces inefficiency, but may also produce internal or “off-target priming” with the second primer. The inherent “dual recognition” of the nucleic acids of some embodiments increases stringency, an effect which carries over into the quantitation by the molecular identifier element in the MIP structure. A unique molecular tag may be placed at one site in the MIP backbone, but in standard targeted sequencing using a molecular identifier, a random sequence is used in both primers. Also, the methods allow for lower reagent costs since coverage across the genome can be achieved with very few MIPs compared to the hundreds or thousands of multiplexed, PCR primers required for targeted sequencing. Nevertheless, the methods enjoy most, if not all, of the economic and performance advantages that targeted sequencing displays over shotgun or whole genome sequencing methods. To this end, the inventors have developed a single-probe capture method for sequencing ready libraries from input of DNA as low as 200 pg of tissue or circulating genetic material. This method simultaneously assesses>200,000 sites across the genome. Further, the inventors have developed an analysis pipeline to identify methylated regions and patterns that are significantly different between sample types.

In sum, the methods and nucleic acids of some embodiments offer clear advantages over previously described genetic methods. For example, whole genome sequencing and massively parallel signature sequencing generally require costly analysis of large, non-informative portions of the genome; whereas the present methods can produce similar answers using a fraction of the genome, thereby reducing assay costs and time. Other approaches rely on selectively assaying informative portions of the genome. While certain aspects share some similarity, the methods, in some embodiments, use a novel, comprehensive approach for identifying repeat, primer-binding sites that allow for greater assay design parameters (sequence agnostic—for example, not limited to repeat line elements), more candidate primers (e.g., because all potential primers are enumerated), simple, lower cost assays that are specific and sensitive enough for clinical utility, and a greater ability to multiplex.

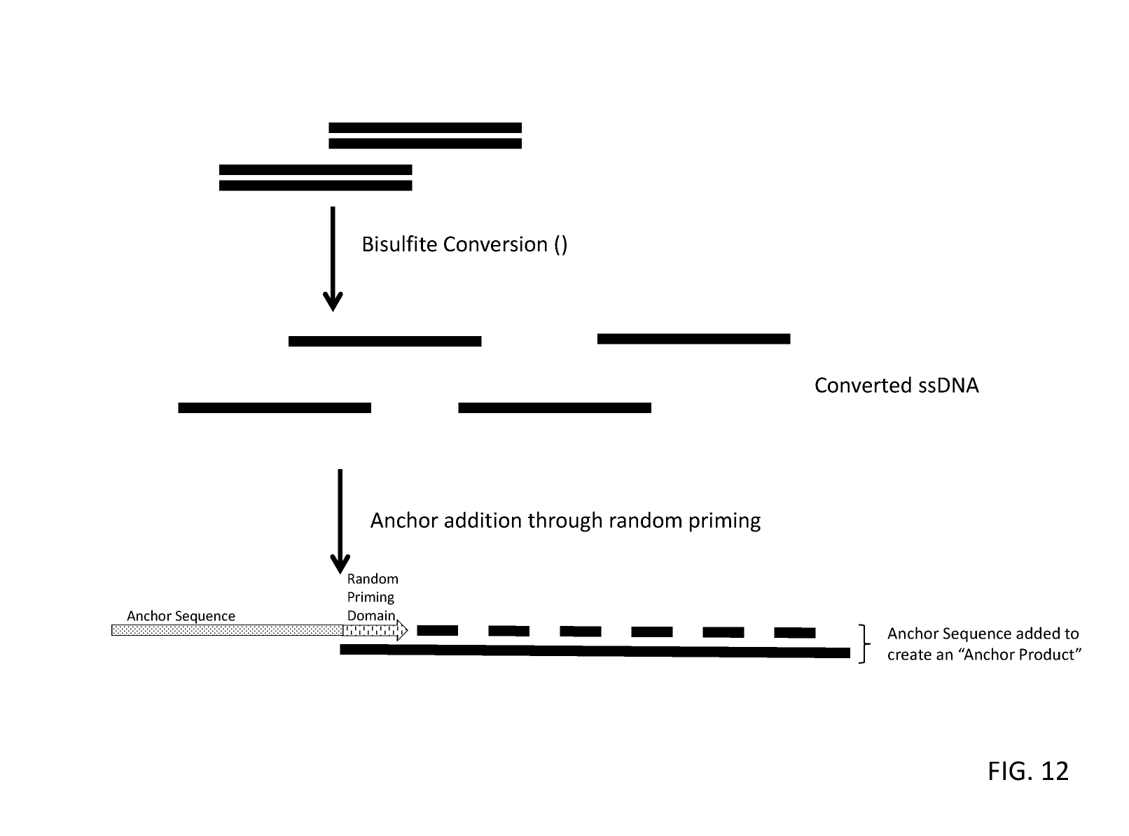

In order to get genome wide methylation information, whole genome bisulfite sequencing techniques are usually employed, which inherently require a large amount of input DNA and large numbers of reads to achieve coverage of desired methylation sites. Embodiments of the present invention provide a solution to the problems of existing methylation detection methods. These embodiments replace previous library preparations with a capture method using a small number of oligonucleotide MIPs comprising targeting polynucleotide arms that hybridize to repeat sequences, said arms being arms attached to high performance universal backbone structures. In some embodiments, these MIPs are designed to flank and incorporate uniquely aligning sequences over the entire human genome, but are enriched for targets pertinent to methylation (i.e., targets containing CpG sites). CpG sites are sites located throughout the genome where methylation occurs at the cytosine nucleotide of the site. By performing sulfonation, hydrolytic deamination and desulfonation (e.g., bisulfite conversion, or simply deamination), unmethylated cytosines are converted to uracils. By contrast, methylated cytosines are protected from this reaction, and, thus, remain cytosines. Therefore, by performing a bisulfite conversion or an alternative procedure that preserves methylated cytosines, and subsequently sequencing CpG sites, the embodiments described herein provide a method of detecting whether a CpG site was methylated or not. Contemplated methods of selecting capture molecules enable the selection of unique sequences in a desired area for quantitation, and do not rely on the presence of some unique sequences in the amplification of convenient repeat sequences. FIG. 12 shows the addition of anchor sequences to single-stranded, bisulfite converted DNA. Rather than adding the anchor sequence via ligation as described previously herein, the anchor sequence can be added to single-stranded nucleic acids, whether bisulfite-treated or not, using random primers that include an anchor sequence. After the anchor sequence is added to the target, the FireMIP assay proceeds as described herein (for example, see FIGS. 3 and 4).

Exemplary applications of the methods include the detection, diagnosis, prognosis, recurrence, minimum residual risk assessment of genetic and epigenetic-associated diseases and conditions. For example, applications might include a method of determining whether a subject has a predisposition to a disease or condition that is associated with the methylation state, mutational landscape, CNV status or fragmentation pattern of a nucleic acid; a method of diagnosing a disease or condition in a subject, said disease or condition being associated with the methylation state, mutational landscape, CNV status or fragmentation pattern of a nucleic acid; a method of detecting the state of a disease or condition in a subject, said disease or condition being associated with the methylation state, mutational landscape, CNV status or fragmentation pattern of a nucleic acid. Particular diseases and conditions include, for example, cancers. A hallmark of cancer cells is that they divide more rapidly than non-cancer cells. Thus, cancer cells and non-cancer cells will have different methylation patterns. The embodiments and methods described herein provide an assay for determining methylation state, mutational landscape, CNV status or fragmentation patterns in tumor biopsy or circulating tumor DNA. In particular, the embodiments and methods can be used to provide a diagnosis, prognosis, staging, and/or likelihood of developing cancers such as, for example, prostate cancer, colorectal cancer, lung cancer, breast cancer, liver cancer, or bladder cancer. Certain embodiments provide a diagnosis, or staging or prognostic information about a cancer, or to inform a treatment decision, or to assess minimum residual risk and recurrence. Conditions known to be affected by methylation, or known to affect methylation, include but are not limited to, aging, diet, lifestyle, ethnicity, development, bipolar disorder, multiple sclerosis, diabetes, schizophrenia, cancer, neurodegenerative diseases, inflammation, lesion, infection, immune response, exposure to: drugs, alcohol, tobacco, pesticides, heavy metals, radiation, UV other environmental factors. In some embodiments, the methods provided herein may provide the methylation status and sequence information of circulating cell-free fetal DNA, for example, as a noninvasive prenatal test. A noninvasive prenatal test using the methods described herein can be used, for example, to determine a risk for preeclampsia or preterm parturition. Additional tests using the methods described herein include pediatric diagnosis of aneuploidy, testing for product of conception or risk of premature abortion, noninvasive prenatal testing (both qualitative and quantitative genetic testing, such as detecting Mendelian disorders, insertions/deletions, and chromosomal imbalances), testing preimplantation genetics, tumor characterization, postnatal testing including cytogenetics, and mutagen effect monitoring.

Another exemplary application of the methods includes a method of differentiating nucleic acid species originating from a subject and one or more additional individuals, said subject and one or more additional individuals having differing methylation states and/or fragmentation patterns of a nucleic acid. For example, the subject may be a pregnant female and the one or more additional individuals may be an unborn fetus. In these embodiments, the blood sample may be maternal plasma or maternal serum. Alternatively, the subject may be a tissue transplant recipient, and the one or more additional individuals may be a tissue transplant donor.

Another exemplary application of the methods includes a method of determining the age or “bio-age” of a subject or group of subjects. More specifically, an individual's genetic material is known to change over time, and the methods described herein allow for the methylation-based age determination of genetic material from an individual by determining the methylation status of thousands or hundreds of thousands of CpG sites in a single assay. This has utility both for forensic purposes and for age-related pathologies such as Alzheimer's disease. As described further in the Examples, the methods are also useful for determining the “bio-age” for specific tissues such as colorectal tissue or the gestational age of a fetus. Combined with the nucleosomal occupancy and/or fragment size analysis, the origin of differentially methylated nucleic acids can be established.

The capture primers and probes (e.g., MIPs) in some embodiments also have the benefit of increased binding stability as compared to conventional PCR primer pairs that are not part of the same molecule. In certain embodiments, the exact targeting arm sequences are somewhat short for PCR primers, and hence will have very low melting temperatures in a PCR context. However, in a MIPs configuration, the primers will enhance binding specificity by cooperating to stabilize the interaction. If one arm has a high binding efficiency, the capture is enhanced even if the opposite arm has a lower efficiency. The additive length of the pair improves the “on/off” equilibrium for capture because the lower efficiency arm is more often in proximity of its target in a MIP than it would be as a free PCR primer.

In some embodiments, a method is provided for determining whether a subject has a predisposition to a disease or condition that is associated with the sequence of a nucleic acid or a population of nucleic acids. In some embodiments, the invention provides a method for diagnosing a disease or condition in a subject, said disease or condition being associated with the genetic or epigenetic profile of a nucleic acid or population of nucleic acids. In some embodiments, the invention provides a method for detecting the state of a disease or condition in a subject, said disease or condition being associated with the genetic or epigenetic profile of a nucleic acid or population of nucleic acids. In certain embodiments, these methods comprise:

a) obtaining a nucleic acid sample isolated from a subject;

b) adding an anchor sequence to one of the 3′ or 5′ end of a plurality of nucleic acids from the sample in step a) to create an anchor product;

c) hybridizing an anchor primer to the ligation product of step b), wherein the anchor primer is substantially complementary to the anchor sequence from step b), and hybridizing a genome-informed primer, which is substantially complementary to a repeat sequence in the nucleic acid, to produce a plurality of replicons, wherein the anchor sequence and the repeat sequence flank a gap region in the plurality of target nucleic acid sequences of interest;

d) sequencing a plurality of amplicons that are amplified from the replicons in step c) to determine the nucleotide sequence of one or more target nucleic acids.

In some embodiments, the size profile of nucleic acids can be determined from the sequence of the different nucleic acid fragments in a population of nucleic acids. In some embodiments, this includes determining the number of capture events (e.g., using unique molecular identifiers) and counting the number of sequences for each uniquely-captured target nucleic acid.

In some embodiments, the mutational landscape of nucleic acids can be determined from the sequence of a population of nucleic acids. In some embodiments, this includes determining the amount or frequency of genetic mutations, which might include single nucleotide variations, deletions, and insertions.

In some embodiments, the presence or absence of gene fusion events can be determined from the sequence of a population of nucleic acids. In some embodiments, this includes determining whether nucleic acids from two different genes are present in a single amplicon.

In some embodiments, the nucleosomal occupancy can be determined from the sequence of a population of nucleic acids, wherein the genome-informed arms bind to protein binding sites and the resulting size and sequence patterns may reveal tissue-specific nucleosomal fragmentation patterns.

In some embodiments, the relative or absolute amount of the different nucleic acid fragments in a population of nucleic acids is determined, thereby informing copy number variant status. In some embodiments, the method further comprises measuring an amount of the amplicons from a sample corresponding to each of a plurality of sizes such that the fractional concentration of different-sized nucleic acids can be determined. Likewise, the fractional concentration of differentially methylated nucleic acids can be determined, or nucleic acids with different mutational landscapes or nucleosomal occupancy patterns. The fractional concentration of nucleic acids can be compared to a reference value to aid in the detection of aberrant nucleic acids.

In some embodiments, a method is provided for determining the methylation status of nucleic acid, wherein a bisulfate conversion step is introduced after step a) and the methylation score of the resulting bisulfate-converted nucleic acid is determined as described herein. In other embodiments, a method is provided of differentiating nucleic acid species originating from a subject and one or more additional individuals, said subject and one or more additional individuals having differing methylation states of a nucleic acid, the method comprising:

a) obtaining a nucleic acid sample isolated from a subject;

b) performing bisulfite conversion of the nucleic acid sample;

c) adding an anchor sequence to the bisulfite-converted nucleic acid of step b);

d) capturing a plurality of target sequences of interest in the nucleic acid sample obtained in step a) by using one or more populations of molecular inversion probes (MIPs) to produce a plurality of replicons,

wherein each of the MIPs in the population of MIPs comprises in sequence the following components:

anchor arm—first unique molecular tag—polynucleotide linker—second unique molecular tag—genome-informed arm;

wherein the anchor arm in each of the MIPs is substantially complementary to the anchor sequence from step c), and the genome-informed arm in each of the MIPs is substantially complementary to a repeat sequence in the nucleic acid, such that the anchor sequence and the repeat sequence flank a unique gap region in the plurality of target sequences of interest;

wherein the first and second unique targeting molecular tags in each of the MIPs in combination are distinct in each of the MIPs;

e) sequencing a plurality of MIPs amplicons that are amplified from the replicons obtained in step d);

f) determining the number of occurrences of cytosine nucleotides at each corresponding known CpG site within the MIPs amplicons sequenced at step e), wherein a methylation status is determined based on the number of occurrences of cytosine nucleotides at each corresponding known CpG site;

g) comparing the methylation status of step f) to the methylation status of one or more other subjects, or a background nucleic acid, to differentiate nucleic acid species.

In alternative embodiments, the bisulfite conversion of any of the methods described herein may be replaced by another type of deamination reaction.

The above methods may also be used to detect aneuploidy in a fetal or non-fetal subject. In some embodiments, fetal aneuploidy and fetal nucleic acid concentration are simultaneously detected in a maternal sample using a combination of CNV status, methylation status, nucleosomal occupancy and size determination, wherein fetal nucleic acid can be differentiated from maternal nucleic acid by any one of methylation status, nucleosomal occupancy or nucleic acid size differences—alone or in combination. In certain embodiments, as an alternative to detecting aneuploidy, the methods can be used to detect and quantify deletions and duplications of genetic features in arms of chromosomes, as well as microscopic deletions and duplications, submicroscopic deletions and deletions, and single nucleotide features including single nucleotide polymorphisms, deletions, and insertions.

In some embodiments, a target nucleic acid species is enriched relative to a background nucleic acid, wherein the target nucleic acid species is differentiated by any one of methylation status, nucleosomal occupancy or nucleic acid size differences, alone or in combination, and thereafter enriched.

In some embodiments, the methods of the invention use a single species of MIP. In alternative embodiments, the methods are useful with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more species of MIPs. For example, multiple species of MIPs can be used to detect different diseases or conditions (e.g., cancer, pregnancy-related conditions such as preeclampsia or preterm parturition, or chromosomal abnormalities such as aneuploidy) in a single sample. In certain embodiments, a single MIP can be used to detect different diseases or conditions (e.g., cancer, pregnancy-related conditions such as preeclampsia or preterm parturition, or chromosomal abnormalities such as aneuploidy) in a single sample.

The skilled worker will appreciate that the lengths and characteristics of the adaptor sequence can be varied as appropriate to ensure it is ligated to the target nucleic acid prior to its capture by the capture probe. For example, the adaptor sequence can be between 20 and 70 bases, e.g., 50-60 bases. In certain embodiments, the anchor sequence, which is included as part of adaptor sequence, is 14, 15, 16, 17, 18, 19, 20, 21, 22, 23, 24, 25, 26, 27, 28, 29, 30, 35, 40, 45 or 50 or more bases. In certain embodiments, the anchor sequence has a melting temperature (T_{M}) between 45° C. and 80° C. (e.g., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C.) and/or a GC content between 10% and 80% (e.g., approximately 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80%). In some embodiments, the sequence of the adaptor sequence is 5′-CATACGAGATCCGTAATCGGGAAGcTGAAGNNNNNNNNNNNGTGAGCTAGTG CT-3′ (SEQ ID NO: 1); and the sequence of the anchor sequence is

(SEQ ID NO: 2)
CATACGAGATCCGTAATCGGGAAGCTGAAG.

In some embodiments, the anchor sequence is added to single-stranded DNA, for example, when the DNA has been bisulfite-converted for subsequent methylation analysis. In certain embodiments, the anchor sequence is added by random priming, and the random primer is a sequence consisting of 4-8 degenerate bases at the 3′ end, for example: NNNN-NNNNNNNN; and the anchor sequence is

(SEQ ID NO: 2)
CATACGAGATCCGTAATCGGGAAGCTGAAG.

Below are examples of the combined random primer sequences with the anchor sequence:

(SEQ ID NO: 3)
CATACGAGATCCGTAATCGGGAAGCTGAAGNNNN
(SEQ ID NO: 4)
CATACGAGATCCGTAATCGGGAAGCTGAAGNNNNNN
(SEQ ID NO: 5)
CATACGAGATCCGTAATCGGGAAGCTGAAGNNNNNNNN

The skilled worker will appreciate that the lengths of the anchor arm and genome-informed arm can be varied as appropriate to provide efficient hybridization between the targeting polynucleotide and the nucleic acid sample. In certain embodiments, the anchor arm or genome-informed arm have a T_{M} between 45° C. and 80° C. and/or GC content between 10% and 80% (e.g., approximately 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80%. In some embodiments, the sequence of the anchor arm is 5′/Phos/CTTCAGCTTCCCGATTACGGATCTCGTATG (SEQ ID NO: 6); and may be hybridized with the below sequence to form a double-stranded adaptor sequence capable of ligating to the target nucleic acid:

(SEQ ID NO: 7)
5′-GCACTAGCTCAC/3PHOS/-3′.

In some embodiments, the sequence of the genome-informed arm is any one of the below sequences, or a sequence that substantially binds to the same genome-informed sites:

(SEQ ID NO: 8)
GAGGCTGAGGCAGGAGAA,
(SEQ ID NO: 9)
AAAACTAAAACAAAAAAA,
(SEQ ID NO: 10)
CTACCNCCNCGCCGA,
(SEQ ID NO: 11)
CTACCNCCNCACAA,
(SEQ ID NO: 12)
GGCCATCTTGGCTCCTCCCCC,
(SEQ ID NO: 13)
AGAAGAATGTATAACTAGAATAACC,
or
(SEQ ID NO: 14)
CCGCGTNGGNGGCAG.

In some embodiments, the sequence of the MIP is any of the following sequences:
m206F—a MIP that binds to the anchor arm with the genome-informed arm being targeted to a ALU element after bisulfite conversion:

(SEQ ID NO: 15)
/5Phos/CTTCAGCTTCCCGATTACGGATCTCGTATGTGTAGATCTCGGT
GGTCGCCGCACGATCCGACGGTAGTGTNNNNNNAAAACTAAAACAAAAAA
A;

or
mCTCF—a MIP that bind to the anchor arm with the genome informed arm being targeted to CTCF consensus sites after bisulfite conversion if the site is methylated:

(SEQ ID NO: 16)
/5Phos/CTTCAGCTTCCCGATTACGGATCTCGTATGTGTAGATCTCGGT
GGTCGCCGCACGATCCGACGGTAGTGTNNNNNNCTACCNCCNCGCCGA;

or
hypomCTCF—a MIP that binds to the anchor arm with the genome-informed arm being targeted to CTCF consensus sites after bisulfite conversion if the site is hypomethylated:

(SEQ ID NO: 17)
/5Phos/CTTCAGCTTCCCGATTACGGATCTCGTATGTGTAGATCTCGGT
GGTCGCCGCACGATCCGACGGTAGTGNNNNNNCTACCNCCNCACAA;

or
mROP208-F—a MIP that binds to the anchor arm with the genome-informed arm being targeted to a ALU element:

(SEQ ID NO: 18)
TCCTACCTCAACCTCCTA(6N)BB(6N)CCAAACTAAAATACAATA,
(SEQ ID NO: 19)
/5Phos/TCCTACCTCAACCTCCTANNNNNNCTTCAGCTTCCCGATTACG
GGCACGATCCGACGGTAGTGTNNNNNNCCAAACTAAAATACAATA;

or
mROP208-R—a MIP that binds to the anchor arm with the genome-informed arm being targeted to a ALU element:

(SEQ ID NO: 20)
CACTACACTCCAACCTAA(6N)BB(6N)CAAAAAACTAAAACAAAA,
(SEQ ID NO: 21)
/5Phos/CACTACACTCCAACCTAANNNNNNCTTCAGCTTCCCGATTACG
GGCACGATCCGACGGTAGTGTNNNNNNCAAAAAACTAAAACAAAA;

or
mROP206-F—a MIP that binds to the anchor arm with the genome-informed arm being targeted to a ALU element:

(SEQ ID NO: 22)
TTCTCCTACCTCAACCTC(6N)BB(6N)CCAAACTAAAATACAATA,
(SEQ ID NO: 23)
/5Phos/TTCTCCTACCTCAACCTCNNNNNNCTTCAGCTTCCCGATTACG
GGCACGATCCGACGGTAGTGTNNNNNNCCAAACTAAAATACAATA;

or
mROP206-R—a MIP that binds to the anchor arm with the genome-informed arm being targeted to a ALU element:

(SEQ ID NO: 24)
CACTACACTCCAACCTAA(6N)BB(6N)AAAACTAAAACAAAAAAA,
(SEQ ID NO: 25)
/5Phos/CACTACACTCCAACCTAANNNNNNCTTCAGCTTCCCGATTACG
GGCACGATCCGACGGTAGTGTNNNNNNAAAACTAAAACAAAAAAA,

wherein “BB” in the above sequences stands for “backbone” and can be a generic backbone or linker sequence.

In some embodiments, the genome-informed arm targets, for example, greater than 1,000, greater than 10,000, greater than 20,000, greater than 30,000, greater than 40,000, greater than 50,000, greater than 60,000, greater than 70,000, greater than 80,000, greater than 90,000, greater than 100,000, greater than 200,000, greater than 300,000, greater than 400,000, greater than 500,000, greater than 600,000, greater than 700,000, greater than 800,000, greater than 900,000, and/or greater than 1,000,000 sequences of interest. In some embodiments, a genome-informed arm does not bind long interspersed nucleotide elements (LINE) in the genome.

Unique molecular tags provide a way to determine the number of capture events for a given amplicon. A MIP may comprise one or more unique molecular tags, e.g., 1, 2, 3, 4, or 5 unique molecular tags. In certain embodiments, the length of the first and/or second unique molecular tag is between 4 and 15 bases, e.g., 4, 5, 6, 7, 8, 9, 10, 11, 12, 13, 14, or 15 bases.

A polynucleotide linker bridges the gap between the two targeting polynucleotide arms (i.e., the anchor arm and the genome-informed arm). In some embodiments, the polynucleotide linker is located directly between the first and second unique molecular tags. In certain embodiments, the polynucleotide linker is not substantially complementary to any genomic region of the subject. In certain embodiments, the polynucleotide linker has a length of between 20 and 1,000 bases (e.g., 20, 25, 30, 35, 40, 45, 50, 55, 60 or 65 bases) and/or a melting temperature of between 45° C. and 85° C. (e.g., 45° C., 50° C., 55° C., 60° C., 65° C., 70° C., 75° C., 80° C., or 85° C.) and/or a GC content between 10% and 80% (e.g., approximately 10%, 15%, 20%, 30%, 35%, 40%, 45%, 50%, 55%, 60%, 65%, 70%, 75%, or 80%). In certain embodiments, the polynucleotide linker comprises at least one amplification primer binding site, e.g., a forward amplification primer binding site. In some embodiments, the linker includes a reverse amplification primer binding site, but the reverse amplification. For example, the sequence of the forward amplification primer can comprise the nucleotide sequence of CCGTAATCGGGAAGCTGAAG (SEQ ID NO: 26) and/or the sequence of the reverse amplification primer can comprise the nucleotide sequence of GCACGATCCGACGGTAGTGT (SEQ ID NO: 27). Thus, the nucleotide sequence of the polynucleotide linker can comprise the nucleotide sequence of

(SEQ ID NO: 28)
CTTCAGCTTCCCGATTACGGGCACGATCCGACGGTAGTGT.

In certain embodiments, the MIP comprises the nucleotide sequence of 5′/Phos/CTTCAGCTTCCCGATTACGGATCTCGTATGTGTAGATCTCGGTGGTCGCCG CACGATCCGACGGTAGTGNNNNNNNGAGGCTGAGGCAGGAGAA-3′ (SEQ ID NO: 29), wherein N represents a randomly generated nucleotide of A, T, C, or Gin each molecular probe. In some embodiments the MIP comprises a 5′ phosphate to facilitate ligation.

In certain embodiments, the MIP is designed with a genome-informed arm to bind particular genomic features, including but not limited to, repeat sites comprising, or in close proximity to, CpG sites, protein binding sites, Alu repeats, gene fusion break points, class switch recombination sites, VDJ recombination sites, D4Z4 repeats, centromeric SAT-alpha repeats, NBL2 repeats, or LINE1 sites.

In some embodiments, the population of MIPs has a concentration between 10 fM and 100 nM, for example, 0.5 nM. In certain embodiments, the concentration of MIPs used will vary with the number of sequences being targeted, e.g., as calculated by multiplying the number of target sequences of interest by the number of genomic equivalents in a reaction (the “total target number”). In particular embodiments, the approximate ratio of the number of MIP molecules to the total target number is 1:50, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1,000. In certain embodiments, each of the MIPs replicons and/or amplicons is a single-stranded circular nucleic acid molecule.

In some embodiments, the MIPs replicons are produced by: i) the genome-informed arm, hybridizing to a first region in the nucleic acid sample, wherein the first region is in proximity to a target sequence of interest; and ii) after the hybridization of the anchor arm and the genome-informed arm, using a ligation/extension mixture to extend and ligate the gap region between the two targeting polynucleotide arms to form single-stranded circular nucleic acid molecules. In certain embodiments, a MIP amplicon is produced by amplifying a MIP replicon, e.g., through PCR.

In some embodiments, the sequencing step comprises a next generation sequencing method, for example, a massively parallel sequencing method, or a short read sequencing method. In some embodiments, sequencing may be by any method known in the art, for example, targeted sequencing, single molecule real-time sequencing, electron microscopy-based sequencing, transistor-mediated sequencing, direct sequencing, random shotgun sequencing, Sanger dideoxy termination sequencing, targeted sequencing, exon sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID® sequencing, MS-PET sequencing, mass spectrometry, and a combination thereof. In some embodiments, sequencing comprises an detecting the sequencing product using an instrument, for example but not limited to an ABI PRISM® 377 DNA Sequencer, an ABI PRISM® 310, 3100, 3100-Avant, 3730, or 373OxI Genetic Analyzer, an ABI PRISM® 3700 DNA Analyzer, or an Applied Biosystems SOLiD™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

A sequencing technique that can be used in various embodiments includes, for example, Illumina® sequencing. Illumina® sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Some embodiments comprise, before sequencing (e.g., the sequencing step of d) as described above), a PCR reaction to amplify the MIPs amplicons for sequencing. This PCR reaction may be an indexing PCR reaction. In certain embodiments, the indexing PCR reaction introduces into each of the MIPs amplicons the following components: a pair of indexing primers comprising a unique sample barcode and a pair of sequencing adaptors. In particular embodiments, the barcoded targeting MIPs amplicons comprise in sequence the following components in a 5′ to 3′ orientation:

a first sequencing adaptor—a first sequencing primer binding site—the first unique targeting molecular tag—the first targeting polynucleotide arm—captured nucleic acid—the second targeting polynucleotide arm—the second unique targeting molecular tag—a second sequencing primer binding site—a unique sample barcode—a second sequencing adaptor. In some embodiments, the sample barcode allows for the testing of multiple samples simultaneously (i.e., multiplexing).

In some embodiments, the target sequences of interest are on a single chromosome. In alternative embodiments, the target sequences of interest are on multiple chromosomes. In particular embodiments, the target sequences of interest are selected at particular sites where methylation status correlates with a disease or condition. In particular embodiments, the target sequences of interest are selected at particular sites where mutations correlate with a disease or condition. In particular embodiments, the target sequences of interest are selected at particular sites where copy number variations correlate with a genomic instability, disease or condition. In particular embodiments, the target sequences of interest are selected at particular sites where protein binding sites correlate with transcription regulation. In particular embodiments, the target sequences of interest are selected at particular sites where gene fusion sites correlate with reactivation of transposons, disease or condition. Because a single MIP sequence can be used to target sequences of interest across an entire genome, in certain embodiments the methods of the invention provide the benefit of being able to detect methylation status of more than one chromosome at a time. By selecting MIPs that provide ample coverage across the genome, the methylation status of the sequences of interest may serve as a proxy for the methylation status of a genome. Moreover, because the MIPs provide sequence information as well as methylation status, the MIPs may be used to detect 1, 2, 3, 4, 5, 6, 7, 8, 9, 10, or more conditions associated with genomic instability, CNV status, mutational landscape, tissue-of-origin, or methylation status and/or chromosomal or other sequence abnormalities.

In some embodiments, the disclosure provides a method of selecting a molecular inversion probe (MIP) from a plurality of candidate MIPs for using to detect CNVs or aneuploidy in a subject, the method comprising:

a) receiving nucleic acid sequences of the plurality of candidate MIPs;

b) for each respective MIP in the plurality of candidate MIPs,
i) computing a first number (A) of unique sites predicted, with no mismatch, to be captured by the respective MIP on a chromosome of interest;
ii) computing a second number (C) of unique sites predicted, with one mismatch, to be captured by the respective MIP on the chromosome of interest;
iii) computing a third number (E) of unique sites predicted, with no mismatch, to be captured by the respective MIP across a genome;
iv) computing a fourth number (G) of unique sites predicted, with one mismatch, to be captured by the respective MIP across the genome;
v) computing a fifth number (F) of non-unique sites predicted, with no mismatch, to be captured by the respective MIP across the genome;
vi) computing a sixth number (H) of non-unique sites predicted, with one mismatch, to be captured by the respective MIP across the genome;
vii) computing a performance metric for the respective MIP based at least in part on the first, second, third, fourth, fifth, and sixth numbers;

c) selecting a MIP, based at least in part on the performance metric computed in step b) vii) for each MIP in the plurality of candidate MIPs.

In some embodiments, the methods provided include a method of selecting a molecular inversion probe (MIP) from a plurality of candidate MIPs for using to detect methylation in a subject, the method comprising:

a) receiving nucleic acid sequences of the plurality of candidate MIPs, wherein each of the MIPs in the plurality of candidate MIPs comprises in sequence the following components:
first targeting polynucleotide arm—first unique molecular tag—polynucleotide linker—second unique molecular tag—second targeting polynucleotide arm;

b) for each respective MIP in the plurality of candidate MIPs,
i) computing a first number (A) of unique CpG sites predicted, with no mismatch on the binding arm sequence, to be captured by the respective MIP;
ii) computing a second number (C) of unique CpG sites predicted, with one mismatch on the binding arm sequence to be captured by the respective MIP;
iii) computing a third number (E) of unique sites predicted, with no mismatch on the binding arm sequence, to be captured by the respective MIP across a genome;
iv) computing a fourth number (G) of unique sites predicted, with one mismatch on the binding arm sequence, to be captured by the respective MIP across the genome;
v) computing a fifth number (F) of non-unique sites predicted, with no mismatch on the binding arm sequence, to be captured by the respective MIP across the genome;
vi) computing a sixth number (H) of non-unique sites predicted, with one mismatch on the binding arm sequence, to be captured by the respective MIP across the genome;
vii) computing a seventh number (I) of CpG sites present on the first targeting polynucleotide arm;
viii) computing an eighth number (J) of CpG sites present on the second targeting polynucleotide arm;
ix) computing a performance metric for the respective MIP based at least in part on the first, second, third, fourth, fifth, sixth, seventh, and eighth numbers;

c) selecting a MIP, based at least in part on the performance metric computed in step b) ix) for each MIP in the plurality of candidate MIPs.

For example, in the methods above, the MIP at step c) may be selected such that a sum of the seventh number (I) and the eighth number (J) is smaller than the corresponding sum for a remaining set of the candidate MIPs. In certain embodiments, a first sum is a sum of the first number (A) and the second number (C), a second sum is a sum of the third number (E), the fourth number (G), the fifth number (F), and the sixth number (H); and the MIP at step c) is selected such that a ratio between the first sum and the second sum is larger than the ratio for a remaining set of the candidate MIPs. In certain embodiments, a third sum is a sum of the third number (E) and the fourth number (G); a fourth sum is a sum of the third number (E), the fourth number (G), the fifth number (F), and the sixth number (H); and the MIP at step c) is selected such that a ratio between the third sum and the fourth sum is larger than the ratio for a remaining set of the candidate MIPs. In certain embodiments, the MIP at step c) is selected based on a ratio (Kₑ) of an average capture coefficient of one mismatch sites (K₁) on the binding arm sequence and an average capture coefficient of zero mismatch sites (K₀):

$${K}_{e} = \frac{{K}_{1}}{{K}_{0}}$$

and wherein the ratio (Kₑ) is experimentally estimated. In certain embodiments, the performance metric at step b) includes a factor corresponding to a weighted sum of the first number (A) and the second number (C). In certain embodiments, the weighted sum corresponds to A+Kₑ× C. In certain embodiments, the performance metric at step b) includes a factor corresponding to a weighted sum of the third number (E) and the fourth number (G). In certain embodiments, the weighted sum corresponds to E+Kₑ×G. In certain embodiments, the MIP at step c) is selected such that a product between a first weighted sum of A+Kₑ×C and a second weighted sum of E+Kₑ×G is larger than the product for a remaining set of the candidate MIPs.

In some embodiments, a nucleic acid molecule comprising a nucleotide sequence of CACTACACTCCAACCTAA (N₁₋₁₀) CTTCAGCTTCCCGATTACGGGCACGATCCGACGGTAGTGT (N₁₁₋₂₀), CAAAAAACTAAAACAAAA (SEQ ID NO: 30), wherein (N₁₋₁₀) represents a first unique molecular tag and (N₁₁₋₂₀) represents a second unique molecular tag, is provided. Additional MIP molecules of the invention include the following:

(SEQ ID NO: 31)
5′/PHOS/CTTCAGCTTCCCGATTACGGATCTCGTATGTGTAGATCTCGG
TGGTCGCCGCACGATCCGTACGGTAGTGTGGCCATCTTGGCTCCTCCCC
C-3′,
(SEQ ID NO: 32)
5′/PHOS/CTTCAGCTTCCCGATTACGGATCTCGTATGTGTAGATCTCGG
TGGTCGCCGCACGATCCGTACGGTAGTGTAGAAGAATGTATAACTAGAAT
AACC-3′,
(SEQ ID NO: 33)
5′/PHOS/CTTCAGCTTCCCGATTACGGATCTCGTATGTGTAGATCTCGG
TGGTCGCCGCACGATCCGTACGGTAGTGTCCGCGTNGGNGGCAG-3′,
(SEQ ID NO: 34)
5′/Phos/CTTCAGCTTCCCGATTACGGATCTCGTATGTGTAGATCTCGG
TGGTCGCCGCACGATCCGACGGTAGTGTNNNNNNAAAACTAAAACAAAAA
AA-3′,
(SEQ ID NO: 35)
5′/Phos/CTTCAGCTTCCCGATTACGGATCTCGTATGTGTAGATCTCGG
TGGTCGCCGCACGATCCGACGGTAGTGTNNNNNNCTACCNCCNCGCCGA-
3′,
(SEQ ID NO: 36)
5′-/Phos/CTTCAGCTTCCCGATTACGGATCTCGTATGTGTAGATCTCG
GTGGTCGCCGCACGATCCGACGGTAGTGTNNNNNNCTACCNCCNCACAA-
3′,
(SEQ ID NO: 37)
5′/Phos/CTTCAGCTTCCCGATTACGGATCTCGTATGTGTAGATCTCGG
TGGTCGCCGCACGATCCGACGGTAGTGTGAGGCTGAGGCAGGAGAA-3′

As described above, in particular embodiments, a) the length of the first unique molecular tag is between 4 and 15 bases; and/or b) the length of the second unique molecular tag is between 4 and 15 bases.

### Methods for Identifying MIPs

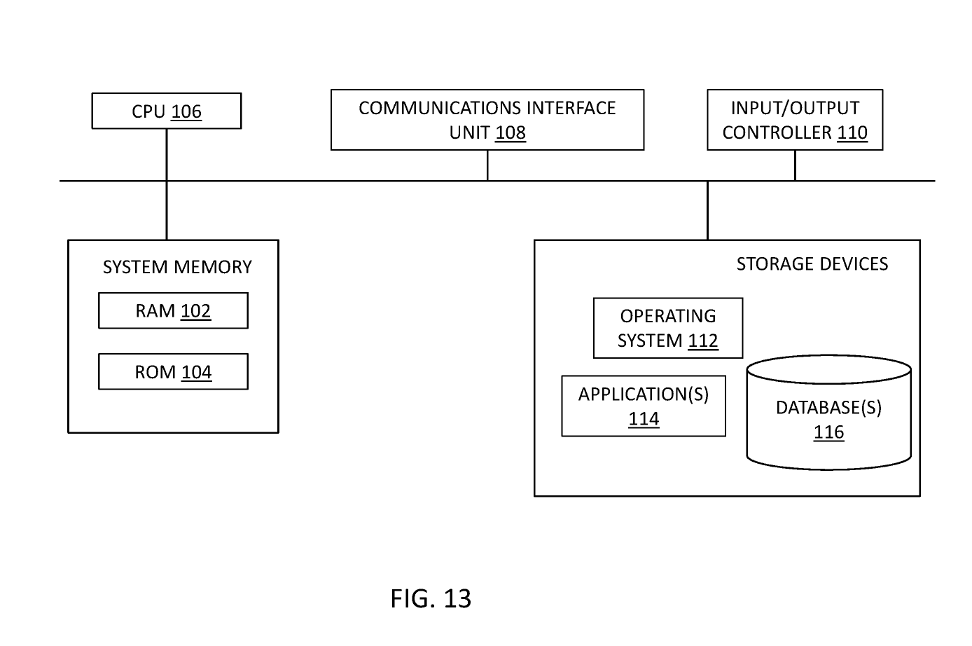

FIG. 13 is a block diagram of a computing device 100 for performing any of the processes described herein, including processes 200, 300, and 500. As used herein, the term “processor” or “computing device” refers to one or more computers, microprocessors, logic devices, servers, or other devices configured with hardware, firmware, and software to carry out one or more of the computerized techniques described herein. Processors and processing devices may also include one or more memory devices for storing inputs, outputs, and data which is currently being processed. The computing device 100 may include a “user interface,” which may include, without limitation, any suitable combination of one or more input devices (e.g., keypads, touch screens, trackballs, voice recognition systems, etc.) and/or one or more output devices (e.g., visual displays, speakers, tactile displays, printing devices, etc.). The computing device 100 may include, without limitation, any suitable combination of one or more devices configured with hardware, firmware, and software to carry out one or more of the computerized techniques described herein. Each of the components described herein may be implemented on one or more computing devices 100. In certain aspects, a plurality of the components of these systems may be included within one computing device 100. In certain embodiments, a component and a storage device may be implemented across several computing devices 100.

The computing device 100 comprises at least one communications interface unit 108, an input/output controller 110, system memory, and one or more data storage devices. The system memory includes at least one random access memory (RAM 102) and at least one read-only memory (ROM 104). All of these elements are in communication with a central processing unit (CPU 106) to facilitate the operation of the computing device 100. The computing device 100 may be configured in many different ways. For example, the computing device 100 may be a conventional standalone computer or alternatively, the functions of computing device 100 may be distributed across multiple computer systems and architectures. In FIG. 13, the computing device 100 is linked, via network or local network, to other servers or systems.

The computing device 100 may be configured in a distributed architecture, wherein databases and processors are housed in separate units or locations. Some units perform primary processing functions and contain at a minimum a general controller or a processor and a system memory. In distributed architecture embodiments, each of these units may be attached via the communications interface unit 108 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 106 comprises a processor, such as one or more conventional microprocessors and one or more supplementary co-processors such as math co-processors for offloading workload from the CPU 106. The CPU 106 is in communication with the communications interface unit 108 and the input/output controller 110, through which the CPU 106 communicates with other devices such as other servers, user terminals, or devices. The communications interface unit 108 and the input/output controller 110 may include multiple communication channels for simultaneous communication with, for example, other processors, servers or client terminals.

The CPU 106 is also in communication with the data storage device. The data storage device may comprise an appropriate combination of magnetic, optical or semiconductor memory, and may include, for example, RAM 102, ROM 104, flash drive, an optical disc such as a compact disc or a hard disk or drive. The CPU 106 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 106 may be connected to the data storage device via the communications interface unit 108. The CPU 106 may be configured to perform one or more particular processing functions.

The data storage device may store, for example, (i) an operating system 112 for the computing device 100; (ii) one or more applications 114 (e.g., computer program code or a computer program product) adapted to direct the CPU 106 in accordance with the systems and methods described here, and particularly in accordance with the processes described in detail with regard to the CPU 106; or (iii) database(s) 116 adapted to store information that may be utilized to store information required by the program.

The operating system 112 and applications 114 may be stored, for example, in a compressed, an uncompiled and an encrypted format, and may include computer program code. The instructions of the program may be read into a main memory of the processor from a computer-readable medium other than the data storage device, such as from the ROM 104 or from the RAM 102. While execution of sequences of instructions in the program causes the CPU 106 to perform the process steps described herein, hard-wired circuitry may be used in place of, or in combination with, software instructions for embodiment of the processes of the present invention. Thus, the systems and methods described are not limited to any specific combination of hardware and software.

Suitable computer program code may be provided for performing one or more functions as described herein. The program also may include program elements such as an operating system 112, a database management system and “device drivers” that allow the processor to interface with computer peripheral devices (e.g., a video display, a keyboard, a computer mouse, etc.) via the input/output controller 110.

The term “computer-readable medium” as used herein refers to any non-transitory medium that provides or participates in providing instructions to the processor of the computing device 100 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 106 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 100 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

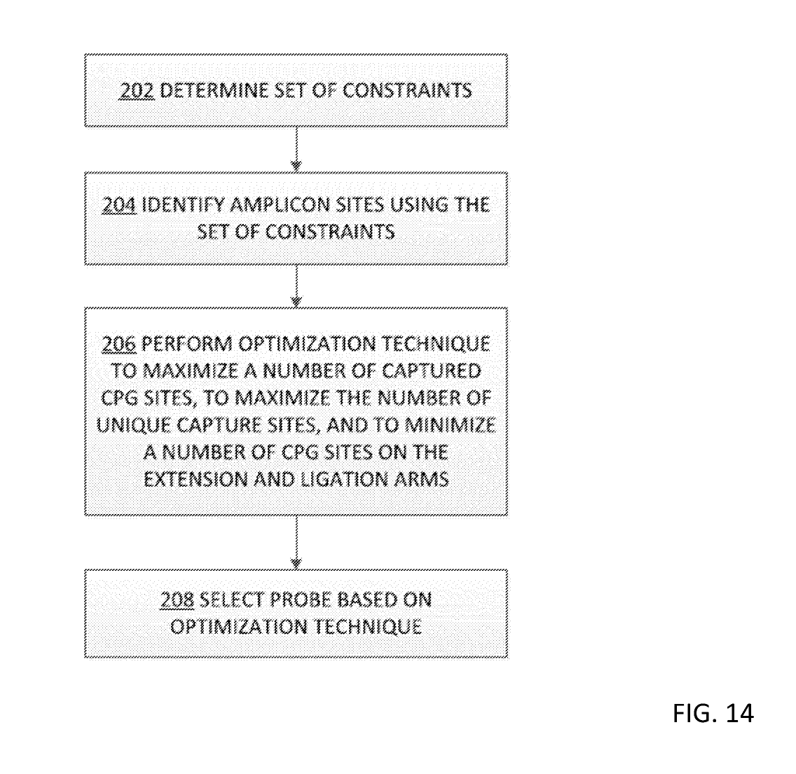

FIG. 14 is a flowchart of a process 200 for designing and selecting a probe (e.g., a MIP), according to an illustrative embodiment for determining methylation status. The process 200 includes the steps of determining a set of constraints (step 202), identifying genome-informed arms using the set of constraints (step 204), performing an optimization technique to minimize a number of CpG sites on the genome-informed arms of the MIP, maximize a total number of captured CpG sites, and maximize a number of uniquely mappable sites (step 206), and selecting a probe based on the optimization technique (step 208). As used herein, “primer” can refer to the hybridizing portion of a capture probe such as a molecular inversion probe. For example, “primer” can refer to the genome-informed arm of a MIP.

At step 202, a set of constraints is determined. The set of constraints may be determined, for example, by CPU 106 using software or application(s) implemented thereon. In some embodiments, the software or application(s) may also be used by CPU 106 to perform any one or more of the subsequent steps in process 200. For example, the software and application(s) may be used by CPU 106 to find abundant repeat sites that bind to the genome-informed arm in a given reference genome (e.g., HG19) based on the determined constraints, and to automatically create suffix-array-based index for the genome file.

In some embodiments, the set of constraints may alternatively be referred to as algorithm flags. For example, the constraints (or algorithm flags) may include a length of the anchor primer or arm and/or the genome-informed primer or arm, a minimum frequency of the primer-pair, a maximum distance between primers (e.g., amplicon length), a minimum and/or maximum total frequency of the primer, a minimum GC-content per primer in percent, a minimum amount of non-identical amplicons in percent, a distribution of primers, or just the genome-informed arm in the genome, or any suitable combination thereof. In an illustrative embodiment, the following constraints may be used in designing primers or probes (e.g., the genome-informed arm):
Length of the genome-informed arm: 10, 11, 12, 13, 14, 15, 16, 17, 18, 19, 20, 21, 22, 23, 24, 25, 26, 27, 28, 29, 30, 31, 32, 33, 34, 35 bases
Frequency of the genome-informed arm: 100, 250, 500, 2500, 5000, 10,000, 100,000, 500,000, 1,000,000
Amplicon Length: 50-150 base pairs, e.g., less than 85 base pairs
Minimum GC content per primer: 10%, 15%, 20%, 30%, 40%
Amplicon uniqueness (percent of target sequences of interest that are unique): greater than about 40%
Distribution of genome-informed arm in genome: iteratively ran, with each bucket size (bs) ranging from 1 to 50%, and bucket-fill (bf) ranging from 1 to bs-1, wherein bucket size (bs) refers to bs % of genome long, and each bucket must contain bf % of all hits.

At step 204, a set of primers are identified using the set of constraints determined at step 202. In particular, for each primer design, any combination of the following parameters may be provided: the genome-informed primer sequences (e.g., as well as the number of their occurrences on the positive and negative strands of the genome), the frequency of the genome-informed primer, the frequency and percentage of the uniquely occurring amplicons, and the amplicon sequences from unique and non-unique pairs. In some embodiments, the anchor primer and the genome-informed primer may be able to amplify multiple regions on the genome (e.g., more than hundreds, more than thousands, more than tens of thousands, more than hundreds of thousands, or more than millions). In some embodiments, multiple MIPs comprising different genome-informed arms maybe used in multiplexed assays to interrogate different parts of the genome (e.g., regions susceptible to gene fusion events, protein binding sites, regions with a high frequency of mutations).

In some methylation-based embodiments, the predicted primer pairs are converted to target bisulfate converted genome. The generated primer pairs may identify or predict amplicon sites without allowing for any mismatches to occur in either the left primer sequence or in the right primer sequence (i.e., the left or right arms) on bisulfite converted genome. Alternatively, in order for additional amplicon sites to be identified or predicted, a small number of mismatches may be allowed, such as allowing for:
0 mismatches in the genome-informed arm
1 mismatch in the genome-informed arm
2 mismatches in the genome-informed arm, or
0 mismatches in the genome-informed arm.

In some embodiments, the amplicon prediction scheme described above provides the genomic coordinates of the predicted amplicons in the genome or the bisulfite converted genome. However, in some embodiments, it may be computationally intensive for the scheme that identifies the amplicon sites without allowing for any mismatches to occur to also provide the genomic coordinates of the predicted amplicons. In this case, the scheme may be divided into two parts. In a first part, the amplicon sites are identified without allowing for any mismatches to occur, and the genomic coordinates of the identified amplicon sites are not provided. In a second part, the amplicon sites that include a small number of mismatches (e.g., the set of mismatches enumerated above) are identified, and the genomic coordinates of these amplicon sites are provided, as well as the genomic coordinate of the no-mismatch amplicon sites. Splitting up the scheme into these two modular parts may save computational complexity. However, in general, it will be understood that the two parts may be combined to provide the set of no-mismatch amplicon sites, mismatch amplicon sites, and their genomic coordinates in a single function.

In some embodiments, one or more of the amplicon sites identified at step 204 may be removed (e.g., by a filtering operation). For example, in some embodiments, arm sequences containing CG dinucleotides are removed. The amplicon sites of those primers that passed the filtering operation (hereinafter referred to as “candidate primers”) should target multiple regions of the reference genome (e.g., typically 2500 or more). Additionally, in some embodiments, both the left and right arm sequences of the candidate primers should have melting temperatures (T_{M}) ranging from 40° C. to high 60° s C as computed by the nearest neighbor model of DNA binding stability, wherein empirical stability parameters are summed according to the nucleic acid sequence. See, e.g., Santa Lucia and Hicks 2004.

After the removal (or filtering) operation, the remaining amplicon sites will be further processed in order to generate a set of parameter values for each candidate primer. In some embodiments, the proportion of the number of amplicon sites coming from a region of interest (e.g., the number of CpGs, the number protein binding sites, the frequency of mutations, the number of gene fusion break points) and the total number of amplicon sites that have passed the filtering operation will be calculated. For each candidate primer, the enrichment information (e.g., the calculated proportion), the associated amplicon sites information, and any other parameter values may be saved in a database, such as database 116.

At step 206, an optimization technique is performed to identify a primer with an optimal predicted performance. The optimization technique involves evaluating an objective function for each candidate primer. In particular, it may be desirable to use an objective function that minimizes a number of CpG sites on the extension and ligation arms of the MIP, maximizes a total number of captured CpG sites, maximizes a number of uniquely mappable sites, or any suitable combination thereof.

The objective function for each candidate MIP may, in some embodiments, be established based on the following matrices:

TABLE 1
Predicted CpG counts from predicted sites

# CpG counts

| | Unique | Non-unique |
|---|---|---|
| 0 mismatch | A | B |
| 1 mismatch | C | D |

TABLE 2
Predicted site count across the genome

Site Counts

| | Unique | Non-unique |
|---|---|---|
| 0 mismatch | E | F |
| 1 mismatch | G | H |

TABLE 3
Number of CpG counts on each arm

# CpG counts

| | Extension arm | Ligation Arm |
|---|---|---|
| # CpG counts | I | J |

In the probe matrices above, rows labeled as “0 mismatch” indicates MIPs with perfect matches in both arms, and rows labeled as “1 mismatch” indicates primers that tolerates at most 1 mismatch in one of its arms in reference to bisulfate converted genome. Several intuitive objective functions can be readily deduced from these probe matrices. For example, an objective function that minimizes I+J (e.g., the sum of I and J is 0) would ensure that probe performance is not a function of an individual's methylation status (e.g., because there are CpG sites present in the arm sequence binding sites. In a second example, an objective function that maximizes (A+C)/(E+F+G+H) may produce reads that specifically target CpG sites. As a third example, an objective function that maximizes (E+G)/(E+F+G+H) selects primers that have significantly more unique capture sites than non-unique capture sites in the genome, or the bisulfate-converted genome. To further illustrate this concept, three exemplary objective functions are explained in detail below.

### A. Total Number of CpG Sites on the Extension and Ligation Arms (P1)

An exemplary objective function for each candidate primer or probe may be defined as the total number CpG sites on the extension arm and ligation arm of the probe:

P1=I+J (1)

### B. Total Number of Useful CpG Sites (P2)

Another exemplary objective function for each candidate primer or probe may be defined as the total number of useful CpG sites that are captured:

P2=g(A,B,C . . . H;K₀,K₁) (2)

where K₀ is the average capture coefficient of 0 mismatch sites and K₁ is the average capture coefficient of 1 mismatch sites. More specifically:

P2=A+KₑC (3)

where Kₑ can be estimated from experimental data, and:

$${K}_{e} = \frac{{K}_{1}}{{K}_{0}} , where 0 < {K}_{e} < 1 \qquad ( 4 )$$

### C. Total Number of Uniquely Mappable Reads (P3)

Another exemplary objective function for each candidate primer or probe may be defined as the total number of uniquely mappable reads across the genome or bisulfite-converted genome:

P3=g(A,B,C . . . H;K₀,K₁) (5)

where K₀ and K₁ are defined above. More specifically, P3 may be defined as:

P3=E+KₑG (6)

where Kₑ is defined in Equation (4).
D. Comprehensive Probe Performance Function A comprehensive way to evaluate an objective function for each candidate primer or probe is to first remove any candidate primer or probe where P1 is not equal to zero. In other words, only candidate primers or probes where P1=0 may be considered, such that no CpG sites on the extension or ligation arms exist. In a second step, a performance function may correspond to:

P=P2×P3 (7)

Incorporating Equations (3) and (6), Equation (7) can be rewritten as:

P=((A+KₑC)×(E+KₑG))/((E+F)+Kₑ(G+H)) (8)

Note that, as described above in relation to Equation (4), the value of Kₑ can be estimated using experimental data. More particularly:

$${K}_{e} = \frac{{K}_{1}}{{K}_{0}} = \frac{\frac{molecular tag counts on 1 mismatch sites}{1 mismatch site count}}{\frac{molecular tag counts on 0 mismatch sites}{0 mismatch site count}} \qquad ( 10 )$$

At step 208, a primer is selected from the set of candidate primers based on the optimization technique performed at step 206. For example, the selected primer may correspond to the primer with the optimal predicted performance, i.e., the primer that had PI=0 and that maximized the objective function as described in relation to step 206.

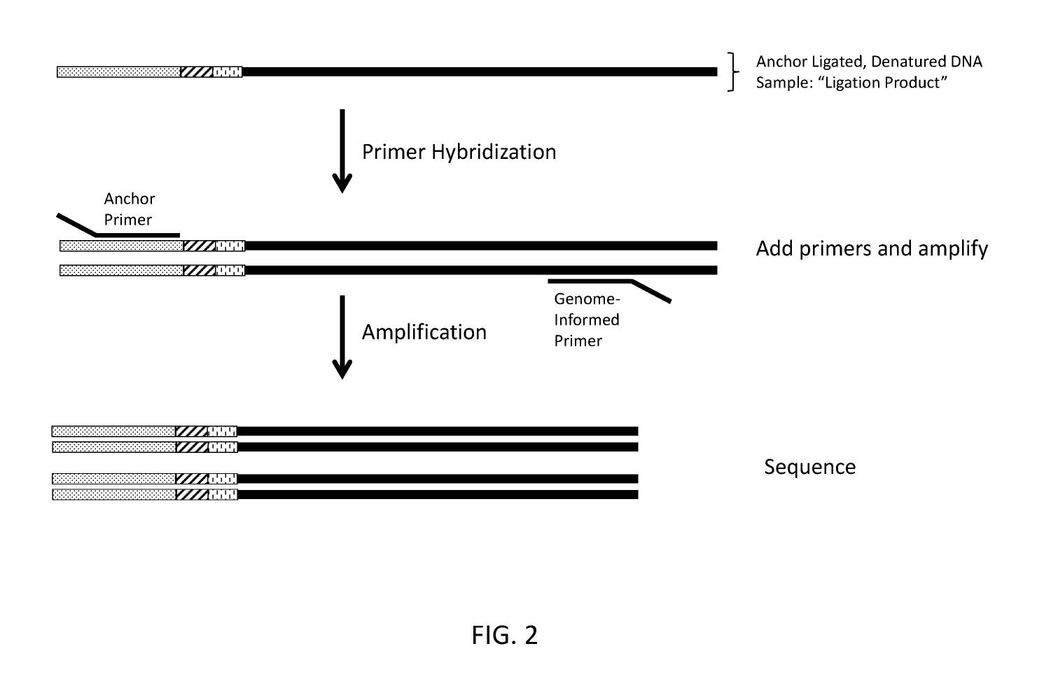

It is contemplated that the steps or descriptions of Process 200 may be used with any other embodiment of this disclosure. In addition, the steps and descriptions described in relation to FIG. 2 may be done in alternative orders or in parallel to further the purpose of this disclosure. For example, each of these steps may be performed in any order or in parallel or substantially simultaneously to reduce lag or increase the speed of the system or method. Furthermore, it should be noted that Process 200 may be carried out using computing device 100, and more particularly, CPU 106 of computing device 100.

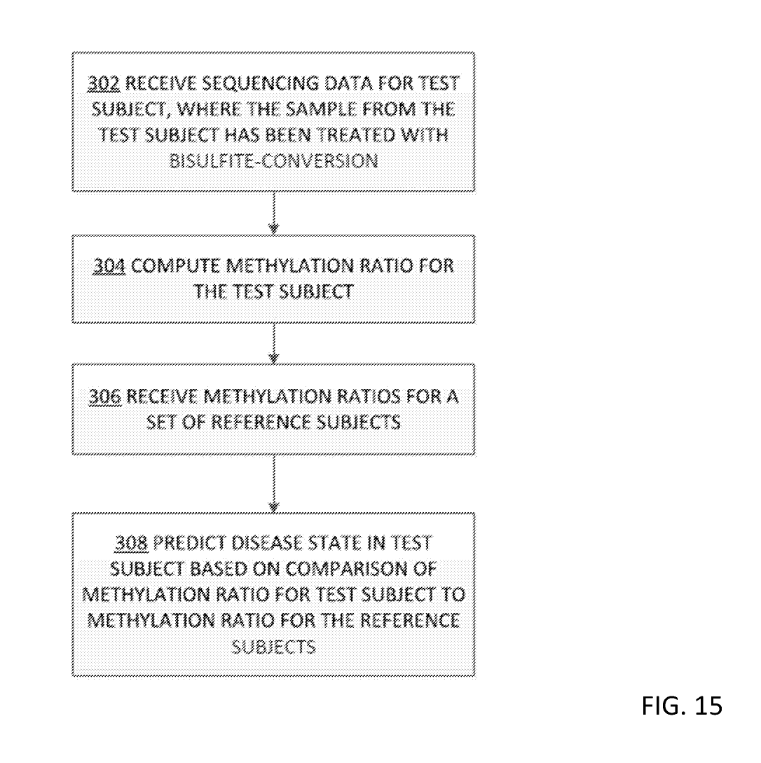

FIG. 15 is a flowchart of a process 300 for predicting a disease state in a test subject, according to an illustrative embodiment. The process 300 includes the steps of receiving sequencing data for a test subject (step 302), computing a methylation ratio for the test subject (step 304), receiving methylation ratios for a set of reference subjects (step 306), and predicting a disease state in the test subject based on comparison of the methylation ratio for the test subject to methylation ratios for the reference subjects (step 308).

The methylation score is calculated from the information contained in the sequencing reads encompassing CpG sites. Every time a CpG site is covered by a read, the information retrieved is considered as a count (methylated or unmethylated) in the formula below. A single read can generate multiple counts if it encompasses multiple CpG sites. Programs like the Bismark Methylation Extractor calculate methylation ratios as follows:

% methylation at CpG sites=100*[Methylated Cs at CpG/(Methylated Cs at CpG+Unmethylated Cs at CpG)]

At step 302, sequencing data for a test subject is received. In particular, the test subject may have a disease state that is unknown, or a predisposition to a particular disease state. The received sequencing data is obtained by obtaining a nucleic acid sample from the test subject, treating the sample with bisulfate conversion, and using a population of primers, such as molecular inversion probes (MIPs), to capture a set of sites in the nucleic acid sample. As is described in detail in relation to FIG. 5, each MIP includes in sequence a first targeting polynucleotide arm, a first unique targeting molecular tag, a polynucleotide linker, a second unique targeting molecular tag, and a second targeting polynucleotide arm. The first and second targeting polynucleotide arms are the same across the MIPs in the population, while the first and second unique targeting molecular tags are distinct across the MIPs in the population. MIPs amplicons result from the capture of the sites, and the amplicons are sequenced to obtain the sequencing data.

At step 304, a methylation ratio is computed for the test subject by evaluating a ratio between a number of methylated cytosine nucleotides within the target regions and a total number of known CpG sites. As is described in detail in relation to FIG. 6, the process of bisulfate-conversion converts un-methylated cytosine nucleotides to uracil nucleotides (which are subsequently converted to thymine nucleotides during PCR), and does not have an effect on methylated cytosine nucleotides. Accordingly, after a sample has been treated with bisulfate-conversion, the presence of remaining cytosine nucleotides at a CpG site indicates that those cytosine nucleotides are methylated. The methylation ratio provides a proportional measure of the methylated cytosine nucleotides, compared to a total number of CpG sites.

At step 306, a set of methylation ratios for a set of reference subjects is received. In particular, the reference subjects may correspond to a group of people that exhibit a known disease state or a known predisposition to have a disease. The methylation ratios for the reference subjects are computed in the same manner as was described in relation to step 304, but for each reference subject.

At step 308, the methylation ratio for the test subject (computed at step 304) is compared to the methylation ratios for the reference subjects (obtained at step 306), and the disease state or predisposition for a particular disease of the test subject is predicted based on this comparison. In particular, a statistical test may be used to compare the test methylation ratio to the population of reference methylation ratios, and determine whether the test methylation ratio belongs in any cluster of reference methylation ratios associated with the same disease state or predisposition.

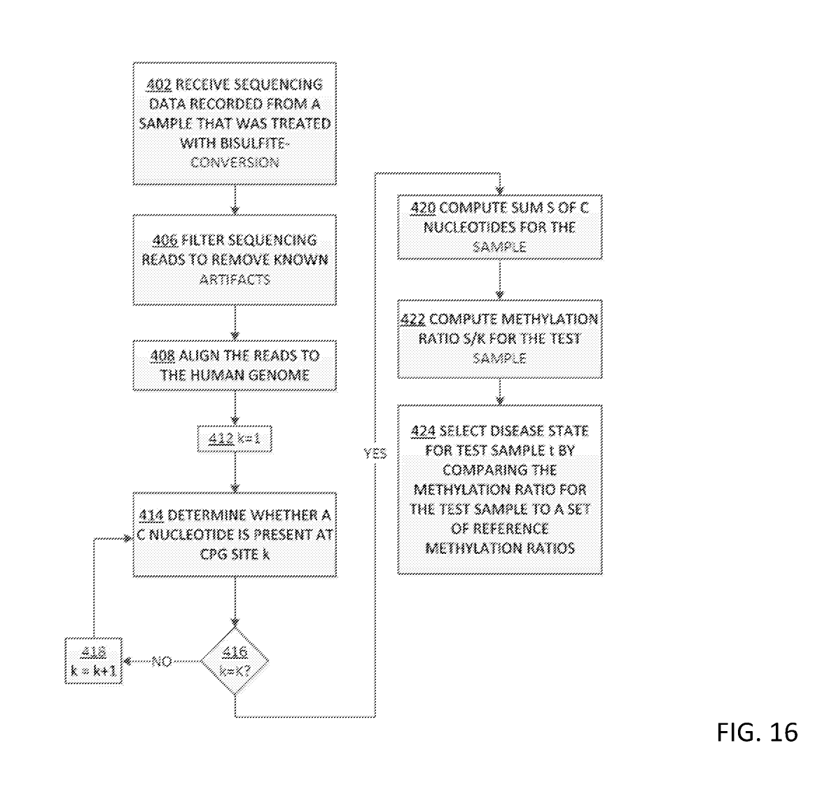

FIG. 16 is a flowchart of a process 400 for predicting a disease state of a test subject, according to an illustrative embodiment. In an example, the process 400 may be used to implement the steps 304 and 308 of the process 300 shown and described in relation to FIG. 15. As was described in relation to FIG. 15, a methylation ratio may be used to predict a disease state in a test subject that has an unknown disease state or a predisposition for a disease.

The process 400 includes the steps of receiving sequencing data recorded from a sample that was treated with bisulfite-conversion (step 402), filtering the sequencing reads to remove known artifacts (step 406), aligning the reads to the bisulfate converted human genome (step 408), setting a CpG site iteration parameter k to 1 (step 412), and determining whether a cytosine nucleotide is present a the k-th CpG site (step 414). When all K CpG sites have been considered, the process 400 further includes the steps of computing a sum S of the numbers of cytosine nucleotides determined at step 414 (step 420), computing a methylation ratio S/K for the test sample (step 422, where K corresponds to a total number of CpG sites), and selecting a disease state for the test sample by comparing the methylation ratio for the test sample to a set of reference methylation ratios (step 424).

At step 402, data recorded from a test sample is received. The test subject has an unknown disease state. The sample may be a nucleic acid sample isolated from the test subject and treated with bisulfate-conversion. The data may include sequencing data obtained from the nucleic acid samples. In an example, the sequencing data is obtained by using a population of MIPs to amplify a set of sites in the nucleic acid sample to produce a set of MIPs amplicons. The MIPs amplicons may then be sequenced to obtain the sequencing data received at step 402.

At step 406, the sequencing reads for the test sample are filtered to remove known artifacts. In one example, the data received at step 402 may be processed to remove an effect of probe-to-probe interaction. In some embodiments, the ligation and extension targeting arms of all MIPs are matched to the paired-end sequence reads. Reads that failed to match both arms of the MIPs are determined to be invalid and discarded. In some implementations, at most one base pair mismatch in each arm is allowed, but any reads that have more mismatches may be discarded. The arm sequences for the remaining valid reads are removed, and the molecular tags from both ligation and extension ends may be also removed from the reads.

At step 408, the resulting trimmed reads are aligned to the human genome. In some embodiments, an alignment tool may be used to align the reads to a reference human genome. In particular, an alignment score may be assessed for representing how well does a specific read align to the reference. Reads with alignment scores above a threshold may be referred to herein as primary alignments, and are retained. In contrast, reads with alignment scores below the threshold may be referred to herein as secondary alignments, and are discarded. Any reads that aligned to multiple locations along the reference genome may be referred to herein as multi-alignments, and are discarded.

At step 412, a CpG site iteration parameter k is initialized to one. The numbers and positions of CpG sites are known.

At step 414, the k-th CpG site is examined to determine whether a cytosine nucleotide is present. As is described in detail in relation to FIG. 6, the process of bisulfate-conversion converts un-methylated cytosine nucleotides to uracil nucleotides (which are later converted to thymine nucleotides during PCR), but does not have an effect on methylated cytosine nucleotides. Accordingly, after a sample has been treated with bisulfate-conversion, the presence of remaining cytosine nucleotides at a CpG site indicates that those cytosine nucleotides are methylated. After the k-th CpG site is examined at step 414, the CpG site iteration parameter k is incremented at step 418 until all K CpG sites have been considered. When all K CpG sites have been considered, the process 400 proceeds to step 420 to compute a sum S of the cytosine nucleotides for the test sample.

At step 422, a methylation ratio S/K is computed for the test sample. The methylation ratio corresponds to the total number of cytosine nucleotides present at the K CpG sites, normalized by K, and provides a proportional measure of the methylated cytosine nucleotides, compared to a total number of CpG sites.

At step 424, the methylation ratio for the test sample is then compared to a set of reference methylation ratios (that have been computed from reference subjects that have known disease states), and a statistical test is performed to select a predicted disease state for the test subject.

The order of the steps in FIG. 16 is shown for illustrative purposes only, and are not limiting.

Since methylation changes are not randomly distributed among the genome, the methylation ratio can be calculated, in some embodiments, by filtering out or isolating targets close to key elements of the genome. For example, to increase the sensitivity of detection of hypomethylation in cancer samples, the targets in proximity to CpG islands can be filtered out since they tend to become hypermethylated. In a second instance, the methylation ratio maybe calculated with targets contained in the intergenic regions since they are known to show higher levels of hypomethylation.

In some embodiments, the level of hypomethylation of a test sample can be determined by comparing its methylation density to a set of control samples (5, 10, 50, 100, 500, 1000, 10,000 or more control samples). The methylation density is defined as the average percentage of methylated C in a CpG context for a defined region or for a defined bin size (1,000, 10,000, 100,000, 1,000,000, 10,000,000 or more bases). For each bin, a Z-score is calculated as follow and the percentage of Zₘₑₜₕ over a defined threshold is determined.

Zₘₑₜₕ=MDₜₑₛₜ−MD_{controls}

MD_{SD-controls}

### Where:

MDₜₑₛₜ is the methylation density for a defined bin for a test sample;
MD_{controls} is the average of the methylation density for a defined bin for a set of control samples; and
MD_{SD-controls} is the standard deviation of the methylation density for a set of control samples.

Also, CNVs, including CNAs, can be calculated the same way by replacing methylation density by read density.

A comparative analysis can also be performed to detect differential methylated CpG sites in a test sample group versus a control group. Methylkit is a R package for DNA methylation analysis (Altuna Akalin, Matthias Kormaksson, Sheng Li, Francine E. Garrett-Bakelman, Maria E. Figueroa, Ari Melnick, Christopher E. Mason. (2012). “methylKit: A comprehensive R package for the analysis of genome-wide DNA methylation profiles.” Genome Biology, 13:R87.) Methylkit can be used to perform sample correlation and clustering, as well as, differential methylation analysis. CpG sites with differential methylation between the test group and the control group can be identified. Some CpG sites may show differential methylation status only in a subset of the test samples. Therefore, identifying a combination of CpG sites with a defined “weight” may be more appropriate to generate an algorithm allowing to evaluate if an unknown samples belong to the tested groups.

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof.

The embodiments referred to above will be better understood from the Experimental Details which follow. However, one skilled in the art will readily appreciate that the specific methods and results discussed are merely illustrative them.

### EXAMPLES

### Example 1: MIP Design and Method for Capturing Target Sequences of Interest

### Probe Design

A single capture probe is created for a semi-redundant site in the genome pertaining to any repeat regions. Additional criteria are designed to target >150,000 sites across the genome with either exact primer match or 1 mismatch. The probe arm melting temperatures is between 45° C. and 75° C.

### Probe Construction

A single oligonucleotide MIP ranging in size between 70-110 bases (depending on the length of the repeat-targeting sequences) is synthesized as shown in FIG. 6. In certain embodiments the single oligonucleotide MIP is between 84-96 bases.

### DNA Preparation

DNA can be extracted from a variety of sources depending on the downstream use, including genomic DNA from whole blood, fragmented plasma DNA or DNA extracted from formalin-fixed paraffin embedded (FFPE) tissues.

### Target Preparation, Capture, and Amplification

### Addition of Anchor Sequence to Fragmented, Double-Stranded DNA:

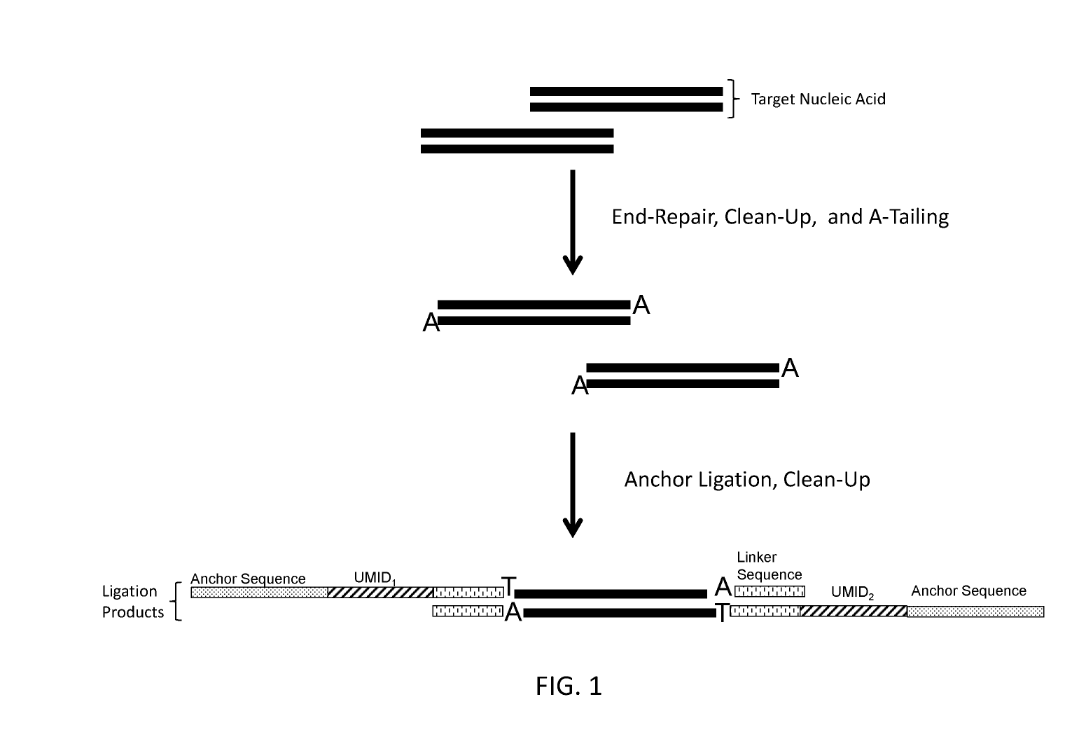

1. End Repair of sheared DNA/plasma DNA for 30 minutes at 30° C. using an End Repair Mix. Bead-based cleanup post reaction (See FIG. 1)
2. A-Tailing of the sheared, end-repaired DNA for 40 minutes using an A-tailing mix. (See FIG. 1)
3. Adapter Ligation using at least a 10 fold molar excess of adapter and an A-T ligation mix for 10 minutes. Bead-based Cleanup post reaction. (See FIG. 1)
   Capture with Molecular Inversion Probe (MIP):
4. FireMIP Hybridization (See FIG. 2)
5. Extension Ligation reaction (See Figure. 2)
6. Exonuclease digestion with Exo I and Exo III (optional)

### Indexing Amplification:

7. Indexing PCR for 25 cycles, which includes P5 adapter, P7 adapter and Sequencing Barcode (See FIG. 3)

### Clean-up, Quantify, Pool and Sequence:

8. Bead-based cleanup
9. Quantify DNA, pool, sequence, for example, using a next generation sequencing method, as described below.

### Sequencing the Captured Sites

Next, the purified PCR products are pooled into a library. The library is sequenced using either single-end or paired-end sequencing, using 75-100 cycles in order to determine the full sequence of the site-specific gap. If single-end sequencing is used, the read will consist of the anchor arm followed by the molecular tag and the unique gap sequence that was filled in during the extension/ligation step. Sequencing into the genome-informed arm is unnecessary because the sequence is known from the probe.

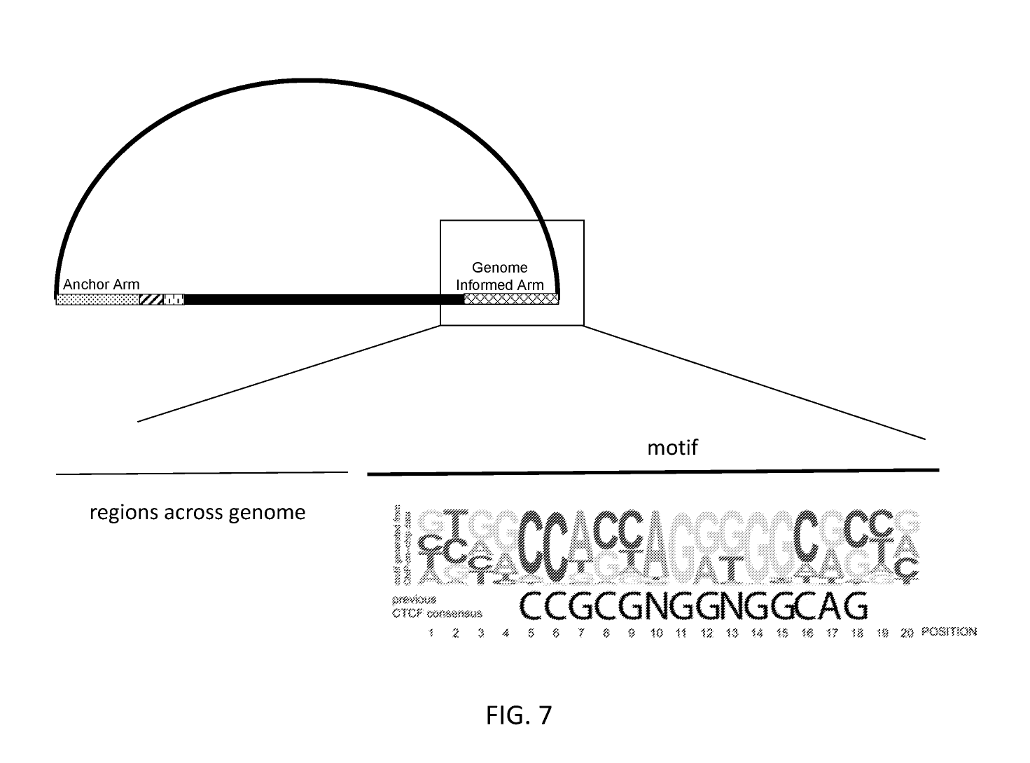

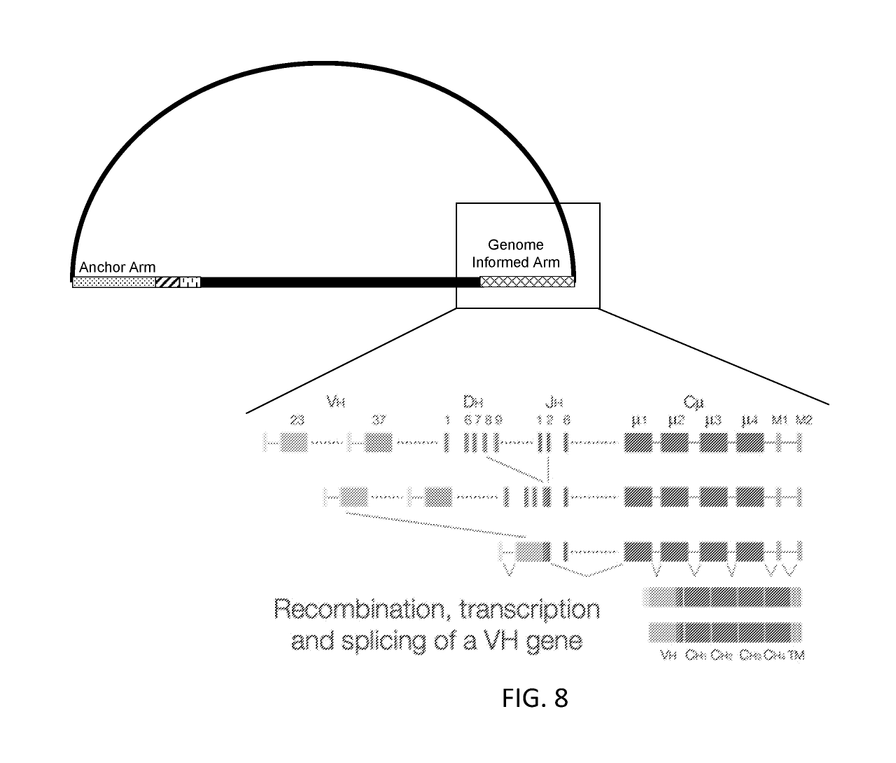

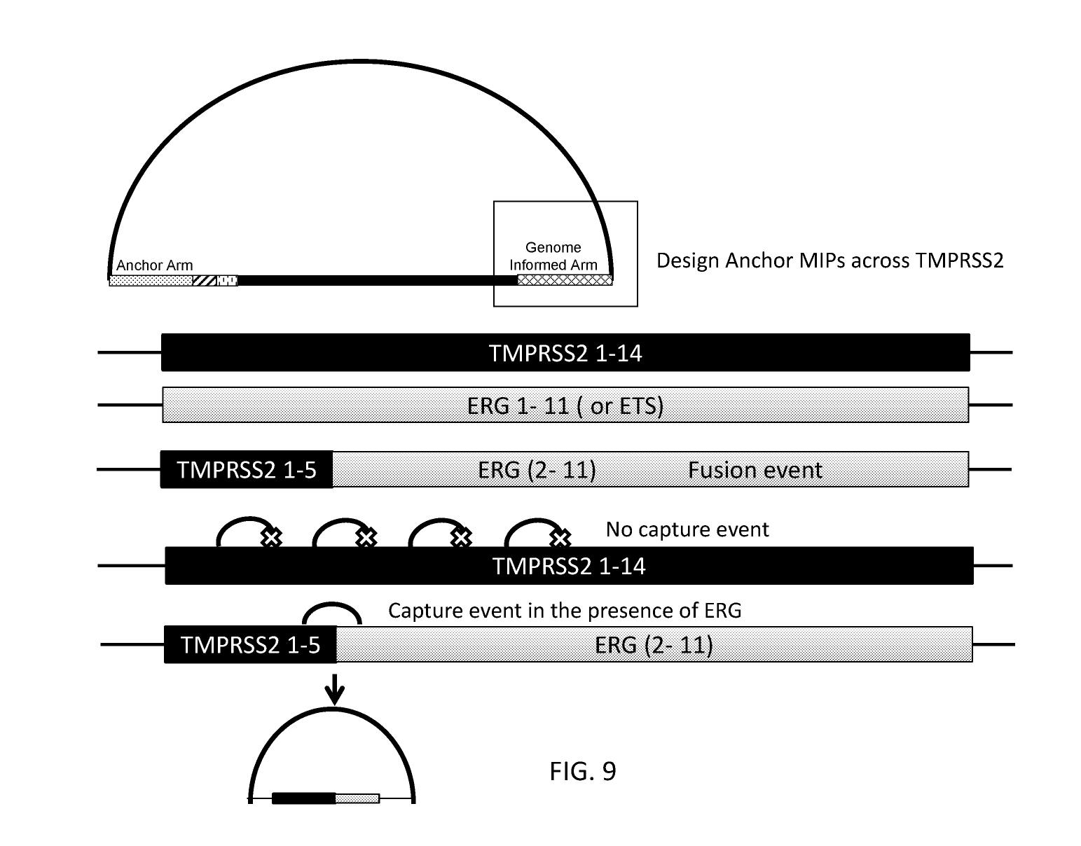

The sequence information can be used to determine the genetic and epigenetic profile of one or more samples. For example, massively parallel sequencing is used to determine the nucleic acid fragment lengths or size profile (see FIG. 5 and FIG. 10), and to identify one or more the methylated pattern in this area (hypo or hyper), the nucleosomal occupancy (see FIG. 7), the immune repertoire (see FIG. 8), the presence or absence of genomic rearrangements like gene fusion events (see FIG. 9), the type and amount of DNA damage (e.g., mutational landscape) incurred, and the count of the sites to assay for large chromosomal abnormalities or genomic instability. As a proof of concept, in FIG. 10 a selected amount of genomic DNA was fragmented via sonication, and the technique described was used. The fragment size of the DNA was measured prior to the method and after. As expected the DNA shows the expected size profile after library preparation, but with a shift of ˜60 bp to reflect the addition of the adaptors.

For methylation analysis, the reads from the sequencer are aligned to an in silico-converted genome to determine positions where C nucleotides are observed instead of the expected T (the bisulfite-conversion produces a U nucleotide, which is read out as a T nucleotide by the sequencing methods). The methylation ratio is calculated as the number of C's observed in CpG sites, divided by the total number CpG dinucleotides in the target sequences of interest. This identifies the ratio of unconverted (i.e., methylated) cytosine nucleotides in the target region. The average methylation ratio of the sample is then reported.

### Removing PCR Duplicates

For improved analysis, PCR duplicates can be removed prior to analysis. The use of unique molecular identifiers allows for each capture event to be characterized. More specifically, these identifiers are used to bin reads resulting from the same capture event, remove duplicates and report a single consensus read.

### Mutational Landscape

As described above, the PCR products generated using the compositions and methods described herein can be sequenced, and the sequence information can be used to measure methylation status, genomic instability, size distribution and also provide a mutational landscape of the genome. All of these measures can be hallmarks of different diseases and conditions, including cancer.

Cancer is a disease of deregulated cell growth caused by damage or alteration to a cell's DNA. As a cell evolves away from a state of regulated homeostasis, it acquires DNA alterations that disrupt key control pathways such as cell cycle regulation, cell death, and energy metabolism.

A more recently appreciated hallmark of cancer is the deregulation of genome stability and DNA repair processes. Deregulation of genome stability can occur via multiple pathways with different cancers having distinctive patterns of instability termed “mutational landscape”. For example, a colorectal tumor in one individual may have a different mutational landscape than a colorectal tumor from someone else. This landscape includes the summation of all single nucleotide substitutions or variations, small insertions and deletions and larger aneuploidies and chromosomal rearrangements. This pattern of mutational landscape has been proposed to differentiate treatment effectiveness in response to immunotherapies. See Rizvi et al. “Mutational landscape determines sensitivity to PD-1 blockade in non-small cell lung cancer” Science; 3 Apr. 2015: Vol. 348, Issue 6230, pp. 124-128.

The ability to detect and categorize the mutational landscape of tumors has clinical value, especially for improved prognosis. The compositions and methods described herein are particularly useful for determining mutational landscape. For example, after capturing and sequencing the DNA of interest, one can align the DNA to the genome using standard or custom methods, and proceed to apply a general variant caller. After application of the variant caller and additional methods to filter variants, the number of transitions, transversions, deletions and insertions can be binned into respective categories and enumerated per megabase of DNA analyzed. Since the location of DNA damage is spread across the genome, one does not need to focus on predetermined, targeted locations. Instead, a technology like that described herein that assays many repeat regions across the genome allows one to elucidate the mutational landscape in a single assay, while also gathering nucleic acid fragment size information to help determine clinical features like tissue-of-origin.

### Example 2: Bioinformatics Workflow

Raw sequencing data must be processed in order for it to be useful in measuring genetic and epigenetic status. To start, sequencing reads are filtered to remove known artifacts such as probe-to-probe interaction, backbone sequences or adapter sequences. The anchor and genome-informed arms of the MIP (i.e., the first and second targeting polynucleotide arms) are then matched to the sequence reads, allowing a maximum of one base pair mismatch in each arm. Reads that fail to meet this criterion are treated as invalid and discarded. At the same time, the molecular tags from both the anchor and genome-informed ends are kept separately for counting of the capture events in a later step—although in some embodiments the tags are kept together. The trimmed reads are aligned to the human genome, or the bisulfite-converted human genome for methylation analysis. The uniquely aligned reads (in sam/bam format files) are examined to count the unique molecular tags for each targeted site with a unique gap sequence. These counts are the initial number of probe-to-target hybridization events that are sequenced in the Next Generation Sequencing (NGS) platform (e.g., an Illumina HiSeq 2500 flowcell). Alternatively or additionally, massively parallel sequencing or sequencing by synthesis may be used. For methylation analysis, the uniquely aligned reads (in bam format files) can be run through the Bismark's Methylation Extractor to determine the methylation status of the sample.

Prophetically, targets or regions that display an aberrantly high level of either technical variation or population baseline variation are screened depending on the disease or condition to give a lower coefficient of variation than could be obtained by random methods of capture and sequencing.

### Example 3: Genomic Instability Analysis in Colorectal Samples

This example describes the use of compositions and methods of the invention to measure the methylation status and genomic instability of adenoma and adenocarcinoma isolated from the colon or the rectum.

Materials Target nucleic acids from colorectal samples are captured and amplified using the anchor arm and genome-informed MIPs and methods described herein.

### Methods

Human genomic DNA (hgDNA) is extracted from fresh frozen tissues from adenomas and adenocarcinomas as well as their corresponding normal adjacent tissues. Allprep DNA/RNA/Protein Mini Kit from Qiagen is used to extract hgDNA following the vendor's manual. The extracted hgDNA is quantified and undergoes bisulfite conversion.

The bisulfite-converted DNA is added to a MIP designed to capture repetitive elements rich in CpG sites from bisulfite converted genome as described herein. First, the MIP anneals to its targets on the bisulfite DNA. After the capture, the annealed probe is extended at its 3′ end by a high fidelity DNA polymerase (see FIG. 3). The extension is stopped when the newly synthetized DNA meets with the anchor arm of the MIP since the DNA polymerase lacks strand displacement activity. The new 3′ end is ligated to the 5′ end of the probe using the energy of the phosphate modification thereby creating a single-stranded circular molecule (or replicon).

After the extension/ligation step, the unligated probes and the gDNA are digested by exonucleases enzymes to remove undesired products in the subsequent PCR amplification reaction.

The PCR reaction is assembled in a final volume of 50ul and PCR performed. The PCR product is cleaned-up and the amplified libraries are quantified. The libraries are pooled at an equimolar ratio at a final concentration of 4 nM.

The libraries are sequenced using a sequencing platform, such as a HiSeq2500. For the HiSeq2500 a fast run mode is used, and custom primers for read 1 and 2, as well as for indexing read, are used. In some embodiments, paired-end reads are generated.

The following exemplary steps are used for sequence analysis: Demultiplexing (retrieving sequencing data from each multiplexed indexed libraries) and FASTQ file generation are performed using Casava (Illumina). Illumina sequencing adapters as well as MIP backbone are trimmed using Trimmomatic (Bolger, A. M., Lohse, M., & Usadel, B. (2014). Trimmomatic: A flexible trimmer for Illumina Sequence Data. Bioinformatics). The reads with more than one mismatch on the anchor arm or the genome-informed arm can be filtered out. Alignment is performed using Bismark, a three letter aligner, (Felix Krueger, Babraham institute) with the bowtie2 option to generate SAM files, then BAM files using Samtools (Li H.*, Handsaker B.*, Wysoker A., Fennell T., Ruan J., Homer N., Marth G., Abecasis G., Durbin R. and 1000 Genome Project Data Processing Subgroup (2009) The Sequence alignment/map (SAM) format and SAMtools. Bioinformatics, 25, 2078-9). The bowtie2 output file contains uniquely aligned reads. A read (or read pair) align uniquely if the alignment has a unique best alignment score. In other words, the reads with multiple best alignment scores are discarded.

### Results

The percentage methylation at CpG context can be calculated and reported by Bismark Methylation Extractor as follows: % methylation at CpG=100*methylated Cs at CpG/(methylated Cs at CpG+unmethylated Cs at CpG). One would expect to see different methylation scores in the tumor tissue vs. the adjacent normal tissue, with the tumor tissue showing relatively less methylation (i.e., hypomethylation).

The level of hypomethylation of the tumor samples is determined by comparing the methylation density of tumor samples and normal samples. The methylation density is defined as the average percentage of methylated C in a CpG context for a defined one megabase bin. To perform this analysis, the coverage files obtained from the Bismark Methylation Extractor are imported into SeqMonk. The methylation density is determined by averaging the methylation status at every megabase bins with a minimum of 25 different counts. Every time a CpG site is covered by a read, the information retrieved is considered as a count. A single read can generate multiple counts if it encompasses multiple CpG. For each bin, Zₘₑₜₕ can be calculated as follows:
Where ZDₜᵤₘₒᵣ is the methylation density in a bin of one megabase for a tumor sample;
MDₙₒᵣₘₐₗ is the average of the methylation density from a bin of one megabase for all of the normal samples; and
MDSD=the standard deviation of the methylation density from all of the normal samples.

The Zₘₑₜₕ is calculated for the valid bins. A bin may be considered hypomethylated if the corresponding Zₘₑₜₕ is below a certain value, for example-5.

### Measuring Genomic Instability

Copy number alterations present in cancer samples can be determined by comparing the read density (RD) from tumor and normal samples. The read density is defined here as the total number of reads found in bins of a defined size, for example, one megabase. First, the reads are normalized for total number of reads to the samples with the highest total number of reads. For example, a total of 1000, 2000, 3000, 4000, or 5000 or more bins can be created from the human genome hg19 (3,137,161,264 bases). The bins with less than 50 reads are removed from the analysis. In some embodiments, the bins from chromosome Y are also filtered out to account for female samples.

Alternatively, specialized software like Nexus 8.0 from BioDiscovery can be used to calculate the copy number variations base on read depth. Nexus 8.0 software can show in detail the CNA events as well as identified cancer related genes positioned at CNAs events.

The genome instability can be reported as the percentage of bins with significant CNAs (gains or losses). This genome instability index is calculated by first determining the read densities at every megabase bin for the tumor and the normal samples as described above. For each bin, Z_{CNA} is calculated as follow:
Where RDₜᵤₘₒᵣ is the read density in a bin of one megabase for a define tumor samples.
RDₙₒᵣₘₐₗ is the average of the read density in a bin of one megabase for all normal samples
RD_{SD}=the standard deviation of the read density from all the normal samples

Different Z_{CNA} are calculated. In some embodiments, Z_{CNA} less than −3 and greater than 3 are considered significantly different than the normal samples. The percentage of bins with significant CNAs can be reported.

Different methylation status at specific bases can also be assessed between the tumor and the normal samples. For example, coverage files are imported into Seqmonk and the methylation status are analyzed for CpG sites with a coverage of at least 30× for the normal and the tumor samples. In some embodiments, 100,000, 150,000, 200,000, 250,000, 300,000, 350,000, or 400,000 or more total CpG sites may meet the criteria for a particular sample. CpG sites that exhibit a significant difference of at least 25%, 30%, 35%, 40%, 45%, 50%, 55%, or 60% or more between the normal and the tumor sample may be reported.

### Example 4: Determining Tissue-of-Origin

The analysis of cell-free DNA (cfDNA) in plasma has been shown to be useful for different diagnostic purposes including, but not limited to, noninvasive prenatal testing and cancer detection or prognosis; however, it has thus far proven difficult to determine the origin or location of different species of cfDNA in a mixture of DNA (e.g., to differentiate plasma cfDNA from hematopoietic cells vs. plasma cfDNA from liver cells) making the clinical utility of cfDNA assays somewhat limited.

Existing methods for determining the tissue-of-origin for cfDNA include the use of tissue-specific RNA expression patterns or tissue-specific methylation patterns. For example, Winston Koh et al. showed the RNA expression patterns of cell-free RNA in plasma can be correlated to certain tissue types (see Koh, et al. PNAS 2014 111 (20) 7361-7366). However, RNA is notoriously unstable, so when measured by RNASeq or RT-qPCR, it has so far proved and non-reliable for clinical use. Using tissue-specific methylation patterns to determine tissue-of-origin has previously relied on whole genome bisulfite sequencing. (See Sun et al. PNAS 2015 112 (40). This has shown tissue-of-origin can be determined from the methylation patterns of the cfDNA at specific loci in the genome. However, in order to get the required signal from whole genome bisulfite sequencing, deep sequencing is required which is time-consuming and expensive.

As described herein, the fractional contributions of a tissue type can be determined using methylation levels of two sets of cell-free DNA molecules, each set being for a different size range and/or a different nucleosomal occupancy profile, to identify a classification of whether the tissue type is diseased. A separation value between the fractional contributions can be compared to a threshold, and a classification can be determined for whether the first tissue type has a disease state based on the comparison. For example, such a technique can identify diseased tissue that releases shorter cell-free DNA molecules by measuring a higher fractional contribution for shorter cell-free DNA molecules than for longer cell-free DNA molecules, or a technique can identify a tissue-specific nucleosomal occupancy profile, for example, by measuring nucleic acid fragment patterns.

### Experiment and Results

Using the compositions and methods described herein, one can determine the contributions of different tissues to a biological sample that includes a mixture of cell-free DNA from different tissues types, whereby one can analyze the methylation patterns, size profiles, and/or nucleosomal occupancy profiles of the DNA mixture. Together or separately, the methylation levels at repeat sites in the genome and/or the nucleic acid size profile and/or the nucleosomal occupancy profile can determine the fractional makeup of various tissue types in the DNA mixture. In some embodiments, the methylation patterns of the tissue types that potentially contribute to the DNA mixture (candidate tissues) can be determined. Then, the methylation pattern of the DNA mixture of interest is determined. For example, methylation levels can be computed at various sites. Since the DNA mixture is composed of the DNA from the candidate tissues, the composition of the DNA mixture can be determined by comparing the methylation patterns of the DNA mixture and the candidate tissue types. Likewise, the size profile of the DNA mixture can be determined by comparing the size profile of the DNA mixture and the candidate tissue types. For example, it is believed cfDNA of apoptotic origin (e.g., from a tumor) is shorter than background cfDNA not of apoptotic origin. A third component, nucleosomal occupancy can be measured and compared to the nucleosomal occupancy profiles of candidate tissue types.

In some embodiments, a separation value (e.g., a subtracted difference or a ratio) in a contribution percentage of a particular tissue type in the DNA relative to a reference value can indicate a disease state. The reference value may correspond to a contribution percentage determined in a healthy individual, and a separation value greater than a threshold can determine a disease state, as the diseased tissue releases more cell-free DNA molecules than healthy tissue.

### Analysis and Results

Generally speaking, the pathway of analysis to determine tissue-of-origin is similar independent of biology or measurement. First, a reference library is generated from an assay that has tissue-specific signals. Next, a deconvolution algorithm is used to interpret unknown samples and provide a percentage estimate of the unknown sample. A clustering analysis or principal component analysis of one or more of the components measured by the assay will show a distinct pattern between the different DNA species.

### Example 5: Determination of Methylation Age

Methylation status is known to change over time with particular tissues becoming hyper- or hypomethylated at different rates depending on a range of factors, including exposure to environmental factors or the presence of disease. Therefore, determining the methylation status as described herein can provide a measure of “bio age”, which may provide an early indication of the presence of age-related pathologies.

### Gestational Age and Fetal Fraction

Using the compositions and methods described herein, the gestational age (GA) can be estimated based on the methylation status of cfDNA from a maternal sample. A global methylation index (GMI) is known to decreases with GA in a linear manner until birth. The increase of hypomethylated DNA is expected as placental DNA increases in abundance as a percentage of total plasma DNA.

The compositions and methods described herein can also be used to determine the fraction of a species of cfDNA in a background cfDNA using differentiating factors such as methylation status, nucleosomal occupancy, and/or nucleic acid size profiles. For example, it has been shown that fetal cell-free nucleic acid can be differentiated from maternal cell-free nucleic acid based on DNA fragment size (see Yu et al., PNAS, vol. 111 no. 23, pgs. 8583-8588 (2014)), and the size profile can be used to determine fetal fraction and/or fetal aneuploidy.

### Example 6: Detection of 5′ Hydroxymethylation

5′hydroxymethylcytosine (5 hmC) originates from the oxidation of 5′ methylcytosine (5 mC). The conversion of 5 mC to 5 hmC is an intermediate step in the active demethylation process. In cells, this reaction is catalyzed by the ten-eleven translocation enzyme family (TET). 5′hydroxymethylcytosine level is often dysregulated in cancer and may contribute to tumor development and progression.

Bisulfite conversion does not distinguish between 5 hmC and 5 mC. Both modifications prevent the conversion of cytosines to uracils. To discriminate 5 hmC from 5 mC using the compositions and methods described herein, gDNA is extracted from a sample and divided into 2 reactions: 1) regular bisulfite conversion and 2) denaturation of gDNA follow by oxidation of 5 hmC to 5-formylcytosine using potassium perruthenate, followed by conversion of 5-formylcytosine to uracil with bisulfite. After MIP capture and sequencing as described herein, the sites of 5 hmC are detected by comparing the data from reactions 1 and 2: In reaction 1, both 5 hmC and 5 mC are found as cytosines, whereas unmethylated cytosines are found as thymines. In reaction 2, 5 mC are found as cytosines but the 5 hmC and unmethylated cytosines are found as thymines. The hydroxymethylation status, as well as the hydroxymethylation density, can be calculated as described herein.

### Example 7: Target Preparation, Capture, Amplification and Sequencing Using ClipMIPs

This example is a representative method for design, and preparation of a probe as well as sequencing a target DNA sample using ClipMIPs.

### Probe Design

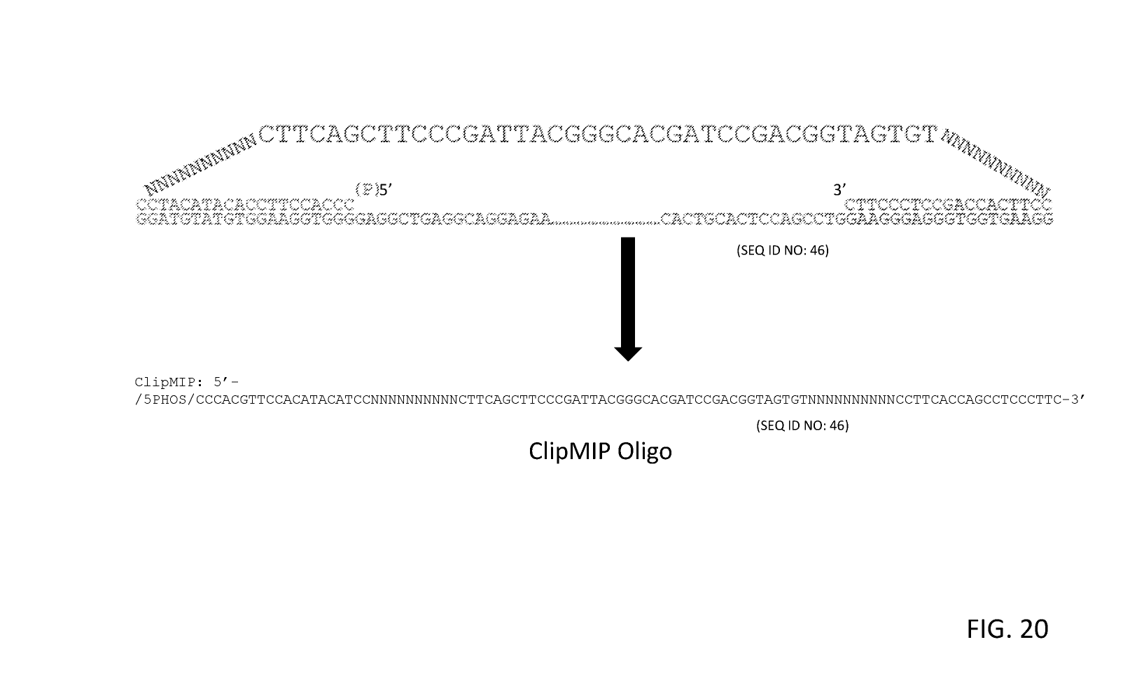

A single capture probe is created that binds to the Clip sequences added to the 5′ and 3′ ends of a DNA fragment. See FIG. 20. The probe arm melting temperature is between 45° C. and 75° C.

### Probe Construction

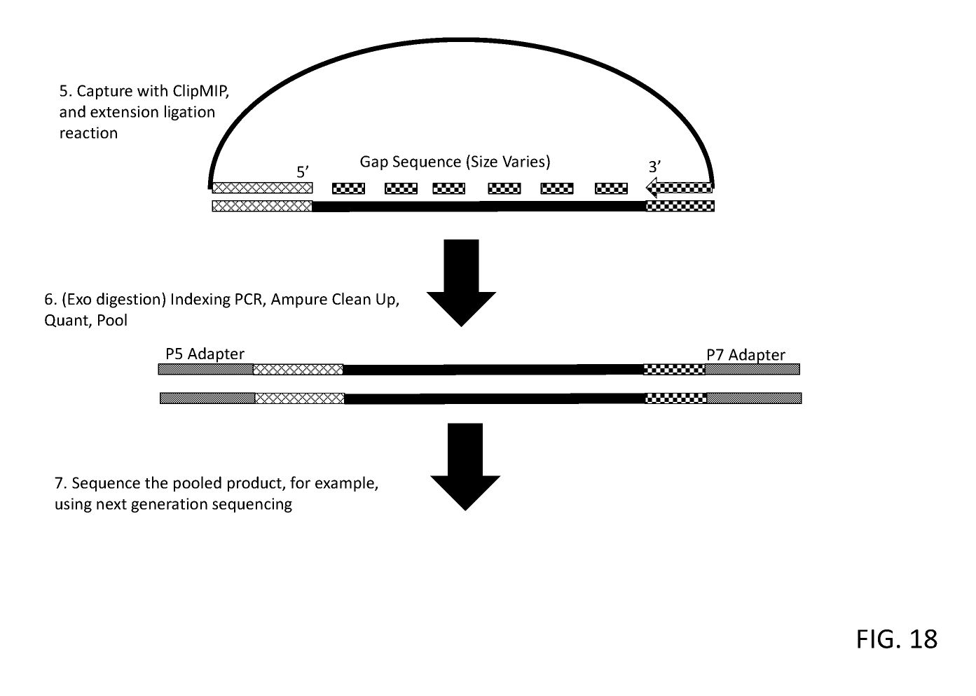

A single oligonucleotide MIP ranging in size between 70-110 bases (depending on the length of the Clip-targeting sequences) comprising Clip binding arms as shown in FIG. 18 and FIG. 20 is constructed. In certain embodiments the single oligonucleotide MIP is between 90-110 bases.

### DNA Preparation

DNA can be extracted from a variety of sources depending on the downstream use, including genomic DNA from whole blood, fragmented plasma DNA (e.g., cell-free DNA) or DNA extracted from formalin-fixed paraffin embedded (FFPE) tissues.

### Target Preparation, Capture and Amplification

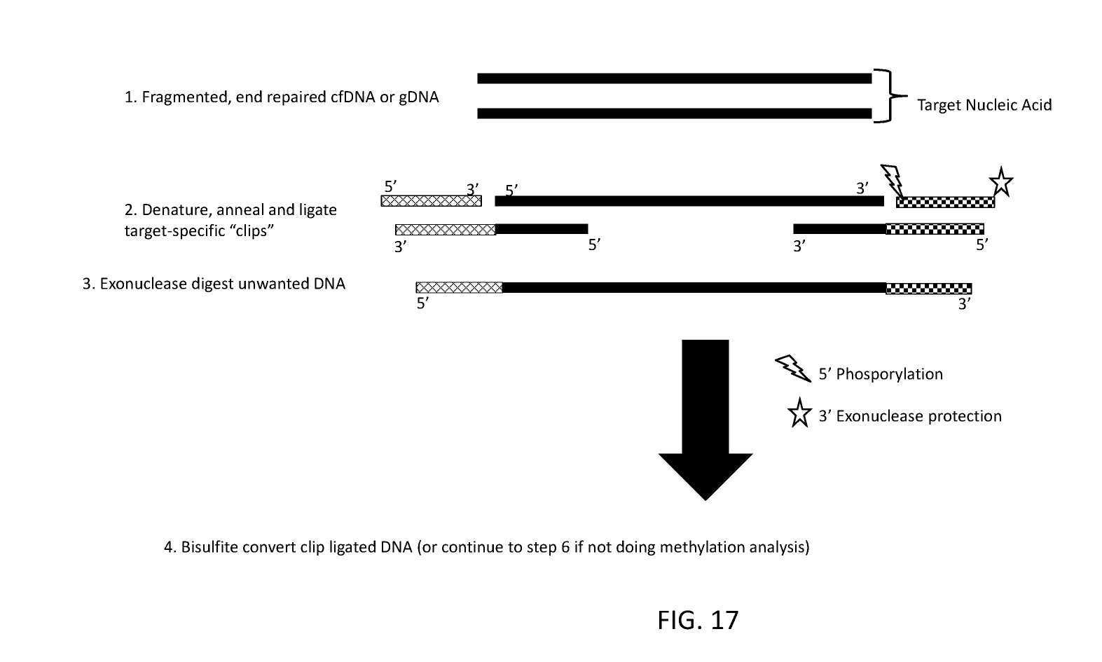

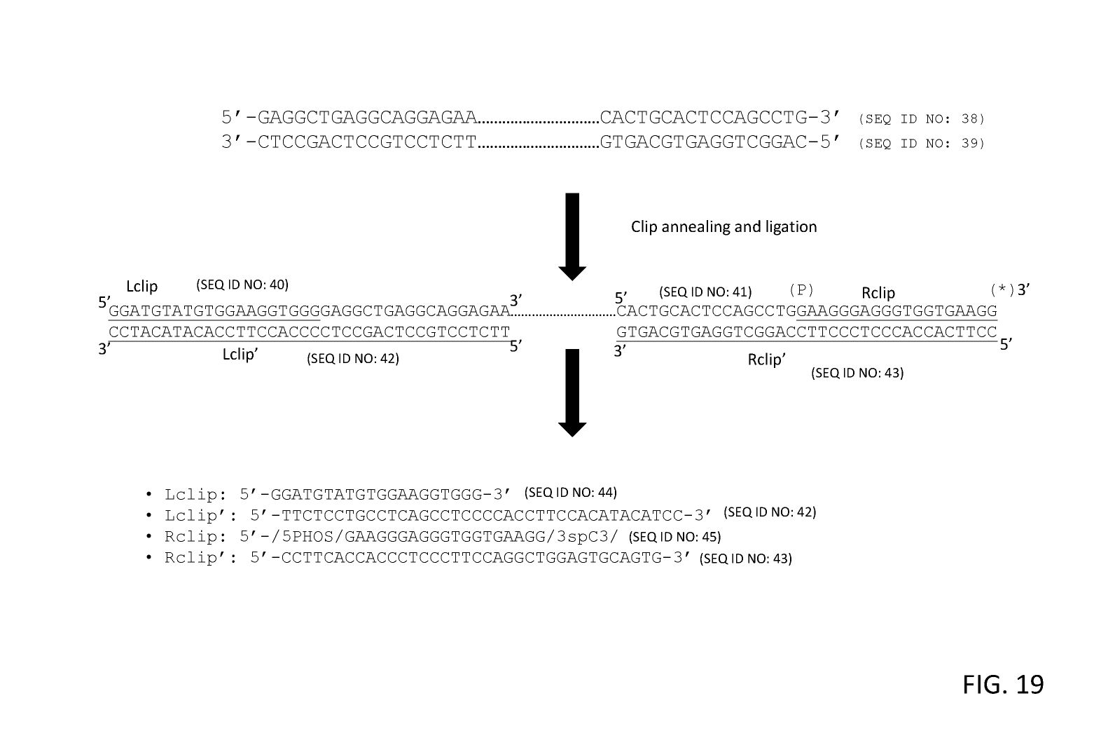

Addition of Clip sequence to fragmented, double-stranded DNA:
1. End Repair of sheared DNA/plasma DNA is conducted for 30 minutes at 30° C. using an End Repair Mix. Bead-based cleanup is conducted post reaction. See FIG. 17.
2. Target nucleic acid is denatured. Clip sequences are hybridized to target (see FIG. 19), followed by an extension/ligation reaction using at least a 10-fold molar excess of target specific adapters and a ligation mix for 10-60 minutes. The Clip sequences are designed not to contain cytosines, thereby allowing for subsequent bisulfite treatment for methylation-analysis. Bead-based cleanup is conducted post reaction. See FIG. 17.
3. In some embodiments, there is an exonuclease digestion step to digest unwanted DNA, in which case 3′ exonuclease protection is included as part of the Clip sequences.
4. Clip ligated DNA is bisulfite converted for methylation-based analysis. Capture with molecular inversion probe (MIP):
5. ClipMIP Hybridization to the target nucleic acid comprising the Clip sequences and subsequent extension ligation reactions are conducted across the gap sequence. See FIG. 18.
6. Exonuclease digestion with Exo I and Exo III (optional) is conducted, followed by the addition of Indexing PCR adapters and Indexing PCR for about 25 cycles. This is followed by an Ampure Clean up, and the products are quantified and pooled.
7. Sequencing, for example, using a next generation sequencing method, is conducted as described below.

### Sequencing the Captured Sites

Next, purified PCR products are pooled into a library. The library is sequenced using either single-end or paired-end sequencing, using 75-100 cycles in order to determine the full sequence of the site-specific gap. If single-end sequencing is used, the read will consist of the first Clip arm followed by the molecular tag and the unique gap sequence that was filled in during the extension/ligation step, the second molecular tag, and the second Clip arm.

The sequenced information can be used to determine the genetic and epigenetic profile of one or more samples. By employing a ClipMIP, hundreds or thousands of unrelated targets can be captured with a single MIP allowing for greatly multiplexed sequencing with a minimal amount of off-target products. For example, massively parallel sequencing can be used to determine the nucleic acid fragment lengths or size profile as described in other related embodiments (see FIG. 5 and FIG. 10), and to identify one or more of the methylated pattern in this area (hypo or hyper), the nucleosomal occupancy (see FIG. 7), the immune repertoire (see FIG. 8), the presence or absence of genomic rearrangements like gene fusion events (see FIG. 9), the type and amount of DNA damage (e.g., mutational landscape) incurred and the count of the sites to assay for large chromosomal abnormalities or genomic instability.

What is claimed is:

1. A method for determining a nucleotide sequence for one or more target nucleic acids of interest in a subject comprising:
a) obtaining a nucleic acid sample isolated from a subject;
b) adding an anchor sequence to one of the 3′ or 5′ end of a plurality of nucleic acids from the sample in step a) to create an anchor product;
c) hybridizing an anchor primer to the anchor product of step b), wherein the anchor primer is substantially complementary to the anchor sequence from step b), and hybridizing a genome-informed primer, which is substantially complementary to a repeat sequence in the nucleic acid, to produce a plurality of replicons, wherein the anchor sequence and the repeat sequence flank a gap region in the plurality of target nucleic acid sequences of interest;
d) sequencing a plurality of amplicons that are amplified from the replicons in step c) to determine the nucleotide sequence of one or more target nucleic acids.

2. The method of claim 1, wherein the plurality of nucleic acids are single-stranded and the anchor sequence is added via random priming.

3. The method of claim 1, wherein the plurality of nucleic acids are double-stranded and the anchor sequence is added via a ligation reaction.

4. The method of any one of claims 1-3, wherein the anchor primer is a hybridization anchor arm on a capture probe, and the genome-informed primer is a hybridization genome-informed arm on the opposite end of the same capture probe.

5. The method of claim 4, wherein the capture probe is a molecular inversion probe (MIP).

6. The method of any one of claims 1-5, wherein the anchor sequence added to the plurality of nucleic acid sequences in step b) further comprises one or more unique molecular tags.

7. The method of any one of claims 1-6, wherein the anchor sequence added to the plurality of nucleic acid sequences in step b) further comprises one or more linker sequences.

8. The method of any one of claims 1-7, wherein the anchor product of step b), further comprises in sequence the following components:
anchor sequence—first unique molecular tag—first polynucleotide linker—captured target nucleic acid—second polynucleotide linker.

9. The method of any one of claims 4-8, wherein the capture probe further comprises one or more unique molecular tags.

10. The method of any one of claims 4-9, wherein the capture probe further comprises a backbone sequence.

11. The method of claim 9 or claim 10, wherein the capture probe further comprises in sequence the following components:
anchor arm—backbone sequence—genome-informed arm.

12. The method of any one of claims 6-11, further comprising a method for determining a number of capture events of each of a population of amplicons of the plurality of amplicons provided in step d) by counting a number of the unique molecular tags of each capture probe that produced a replicon, wherein the population of amplicons is determined by the sequence of the target sequence of interest.

13. The method of claim 12, wherein the number of capture events is indicative of a capture bias.

14. The method of claim 12, further comprising using the number of unique molecular tags to identify duplicates to improve analysis.

15. The method of any one of claims 1-14, further comprising determining a number of the unique amplicons sequenced at step d); determining a read density based on or based in part on the number of unique amplicon sequences; and detecting copy number variation by comparing the read density to a plurality of reference read densities that are computed based on reference nucleic acid samples isolated from reference subjects.

16. The method of claim 15, further comprising determining the number of unique amplicon sequences in defined regions to determine the read density.

17. The method of any one of claims 3-16, wherein the double-stranded plurality of nucleic acids is subjected to end-repair prior to ligation to the anchor sequence.

18. The method of any one of claims 3-16, wherein the double-stranded plurality of nucleic acids is subjected to end-repair and phosphorylation prior to ligation to the anchor sequence.

19. The method of any one of claims 3-16, wherein the double-stranded plurality of nucleic acids is further subjected to end-repair, phosphorylation, and A-tailing prior to ligation to the anchor sequence.

20. The method of any one of claims 17-19, further comprising a bead-based cleanup step after the ligation of the anchor sequence to the plurality of nucleic acids.

21. The method of any one of claims 1-3 and 6-11, wherein the method further comprises, before the sequencing step of d), an extension-ligation step to produce circular replicons.

22. The method of any one of claims 1-21, wherein the method further comprises an exonuclease digestion step that digests non-circular, linear nucleic acids.

23. The method of claim 22, wherein, following the exonuclease digestion, the method further comprises a linearizing step wherein the circular probe is cleaved to become linear.

24. The method of any one of claims 1-23, wherein the method comprises, before the sequencing step of d), a PCR reaction to amplify the replicons thereby producing amplicons for sequencing.

25. The method of claim 24, wherein the PCR reaction is an indexing PCR reaction.

26. The method of claim 25, wherein the indexing PCR reaction introduces into each of the amplicons the following components: a pair of indexing primers, a unique sample barcode and a pair of sequencing adaptors.

27. The method of claim 26, wherein the barcoded amplicons comprise in sequence the following components:
a first sequencing adaptor—a first sequencing primer—an anchor arm hybridizing sequence—a first unique molecular tag—a captured target nucleic acid—a genome-informed arm hybridizing sequence—the second unique molecular tag—a unique sample barcode—a second sequencing primer—a second sequencing adaptor.

28. The method of any one of claims 1-27, wherein the repeat sequence is selected from the group consisting of Alu repeats, protein binding sites, class switch recombination sites, VDJ recombination sites, D4Z4 repeats, centromeric SAT-α repeats, NBL2 repeats, and LINE1 sites.

29. The method of any one of claims 1-28, wherein the target sequence of interest is located in an Alu element.

30. The method of any one of claims 1-29, wherein the target sequence of interest is located in the right arm of an Alu element.

31. The method of any one of claims 1-30, wherein the nucleotide sequence of 50,000 or more different target nucleic acids in a subject is determined using a single capture probe.

32. The method of any one of claims 1-31, wherein the amplicon sequence from step d) is used to determine the size of the amplicon.

33. The method of claim 32, wherein the sizes of 1,000 or more different target nucleic acids in the nucleic acid sample are determined using a single capture probe.

34. The method of claim 32 or 33, wherein at least some of the nucleic acids of interest in the subject are cell-free target nucleic acids, the method further comprising:
a) measuring amounts of amplicons from the nucleic acid sample corresponding to each of a plurality of sizes, the amounts comprising amplicons from cell-free target nucleic acids and from background nucleic acids, thereby measuring amounts of nucleic acids at the plurality of sizes;
b) calculating a first value of a first parameter based on the amounts of nucleic acids at the plurality of sizes, the first parameter providing a statistical measure of a size profile of nucleic acids in the sample;
c) comparing the first value to a reference value; and
d) estimating the fractional concentration of the target nucleic acids among background nucleic acid in the sample based on the comparison of step c).

35. The method of claim 34, wherein the cell-free target nucleic acids are of apoptotic origin.

36. The method of claim 34, wherein the cell-free target nucleic acids are of fetal origin, and the background nucleic acids comprise maternal nucleic acids, whereby the concentration of fetal nucleic acids in a sample from a maternal subject is determined.

37. The method of claim 36, wherein the reference value is from one or more pregnant subjects with known concentrations of fetal nucleic acids.

38. The method of claim 34, wherein the cell-free target nucleic acids are from a tumor, and the background nucleic acids comprise non-tumor nucleic acids, whereby the concentration of tumor nucleic acids in a sample is determined.

39. The method of claim 38, the wherein reference value is from one or more cancer-free subjects.

40. The method of claim 34, wherein the cell-free target nucleic acids are from a donor, and the background nucleic acids comprise host nucleic acids, whereby the concentration of transplanted donor nucleic acids in a sample from the host is determined.

41. The method of any one of claims 4-27, further comprising determining the number of unique amplicon sequences to measure copy number variation, wherein the number of unique amplicons is determined by the sequence of the target sequence of interest determined in step d).

42. The method of claim 41, wherein the number of unique amplicon sequences is compared to a known reference.

43. The method of claim 41, further comprising determining a size distribution of each of a population of unique amplicon sequences.

44. The method of any one of claims 4-27, wherein the one or more target nucleic acids from the sample in step a) comprise one or more sequence mutations that are detected by sequencing step d), thereby determining a mutational landscape.

45. The method of claim 44, wherein the one or more sequence mutations is selected from the group consisting of single nucleotide variations, deletions, insertions, translocations, fusions, and repeat expansions.

46. The method of claim 44, wherein 100 or more different sequence mutations are detected by a single capture probe.

47. The method of claim 44, wherein the frequency or type of sequence mutations is compared to a known reference.

48. The method of any one of claims 4-27, wherein a methylation status of one or more target nucleic acids in a subject is determined, the method further comprising:
a) performing bisulfite conversion of the nucleic acid sample;
b) sequencing amplicons from replicons of the bisulfite converted nucleic acid sample; and
c) determining the number of occurrences of cytosine nucleotides at each corresponding known CpG site within the unique amplicons, wherein the methylation status is determined based on the number of occurrences of cytosine nucleotides at each corresponding known CpG site.

49. The method of any one of claims 1-27, wherein nucleosomal occupancy at one or more target nucleic acids in a subject is determined, the method further comprising:
a) hybridizing a genome-informed primer to a protein binding site in one or more target nucleic acids;
b) determining the size of the plurality of amplicons based on the amplicon sequence from step d), thereby determining an amplicon fragmentation pattern, wherein the fragmentation patterns is indicative of nucleosomal occupancy.

50. The method of claim 49, wherein the protein binding site is a transcription factor binding site or a nuclease binding site.

51. The method of claim 49, further comprising comparing the nucleosomal occupancy to a reference nucleosomal occupancy.

52. The method of any one of claims 1-27, wherein a gene fusion event at one or more target nucleic acids in a subject is detected, the method further comprising:
a) hybridizing a genome-informed primer to a gene-specific sequence in one or more target nucleic acids;
b) determining a presence or absence of a gene fusion event based on the amplicon sequence from step d), wherein the presence of two different gene sequences in a single amplicon is indicative of a gene fusion event.

53. The method of any one of claims 49-52, wherein two or more different genome-informed primers are used in a single, multiplexed assay.

54. The method of any one of claims 1-53, wherein the nucleic acid sample is DNA or RNA.

55. The method of claim 54, wherein the nucleic acid sample is genomic DNA.

56. The method of claim 55, wherein the genomic DNA is fragmented.

57. The method of any one of claims 1-55, wherein the sample is a blood sample selected from a whole blood sample, a plasma sample, and a serum sample.

58. The method of claim 57, wherein the blood sample is a plasma sample.

59. The method of any one of claims 1-58, wherein sequencing information is used in the determination of whether the subject has the predisposition to a disease or condition, or used in diagnosing a disease or condition, or used in detecting a state of a disease or condition, or used in differentiating the nucleic acid species originating from the subject and from one or more additional individuals.

60. A method of detecting copy number variation in a subject, comprising:
a) obtaining a nucleic acid sample isolated from a subject;
b) adding an anchor sequence to one of the 3′ or the 5′ end of a plurality of nucleic acid sequences from the sample in step a);
c) capturing a plurality of target sequences of interest in the nucleic acid sample obtained in step a) by using one or more populations of molecular inversion probes (MIPs) to produce a plurality of replicons,
wherein each of the MIPs in the population of MIPs comprises in sequence the following components:
anchor arm—polynucleotide linker—genome-informed arm;
wherein the anchor arm in each of the MIPs is substantially complementary to the anchor sequence from step b), and the genome-informed arm in each of the MIPs is substantially complementary to a repeat sequence in the nucleic acid, such that the anchor sequence and the repeat sequence flank a unique gap region in the plurality of target sequences of interest;
d) sequencing a plurality of MIP amplicons that are amplified from the replicons obtained in step c);
e) determining a number of a first population of amplicons of the plurality of amplicons provided in step d) based on the number of unique amplicon sequences;
f) determining a number of each of a second population of amplicons of the plurality of amplicons provided in step d) based on the number of unique amplicon sequences;
g) determining, for each target sequence of interest from which the first population of amplicons was produced, a site capture metric based at least in part on the number of capture events determined in step e);
h) identifying a first subset of the site capture metrics determined in step g) that satisfy at least one criterion;
i) determining, for each target sequence of interest from which the second population of amplicons was produced, a site capture metric based at least in part on the number of capture events determined in step f);
j) identifying a second subset of the site capture metrics determined in step i) that satisfy the at least one criterion;
k) normalizing a first measure determined from the first subset of site capture metrics identified in step h) by a second measure determined from the second subset of site capture metrics identified in step j) to obtain a test ratio;
l) comparing the test ratio to a plurality of reference ratios that are computed based on reference nucleic acid samples isolated from reference subjects without a copy number variation at the target sequences of interest; and
m) determining, based on the comparing in step l), whether a copy number variation is present at the target sequences of interest in a subject.

61. The method of claim 60, wherein the nucleic acid sample is isolated from a maternal blood sample comprising fetal nucleic acid, and the copy number variation is a fetal aneuploidy determined by comparing the test ratio to a plurality of reference ratios that are computed based on reference nucleic acid samples isolated from reference subjects known to exhibit euploidy or aneuploidy.

62. The method of claim 60, wherein the nucleic acid sample is isolated from a maternal blood sample comprising fetal nucleic acid, and a fetal aneuploidy is detected, the method further comprising comparing the distribution of maternal and fetal amplicon sequences from the maternal sample to the normal distribution of amplicon sequences from a euploid chromosome from the same sample, whereby a chromosomal copy number variation is indicative of a fetal aneuploidy.

63. The method of claim 60 or 61, wherein the size of one or more amplicons from the plurality of target sequences is determined based on the amplicon sequence from step d).

64. The method of any one of claims 60-63, wherein the site capture metric comprises a site capture efficiency index (SCE).

65. The method of any one of claims 60-64, wherein the site capture metric comprises a site capture consistency measure (SCC).

66. The method of any one of claims 60-65, wherein each of the MIPs replicons provided in step c) is produced by:
i) the anchor arms and genome-informed arms, respectively, hybridizing to the first and second regions in the nucleic acid sample, respectively, wherein the first and second regions flank a target sequence of interest; and
ii) after the hybridization, using a ligation/extension mixture to extend and ligate the gap region between the two arms to form single-stranded circular nucleic acid molecules.

67. The method of any one of claims 60-66, wherein the method comprises, before the sequencing step of d), performing a PCR reaction to amplify the MIP replicons for sequencing.

68. The method of claim 67, wherein the PCR reaction is an indexing PCR reaction.

69. The method of claim 68, wherein the indexing PCR reaction introduces into each of the MIPs amplicons the following components: a pair of indexing primers, a unique sample barcode and a pair of sequencing adaptors.

70. The method of claim 69, wherein the barcoded MIPs amplicons comprise in sequence the following components:
a first sequencing adaptor—a first sequencing primer—the first unique targeting molecular tag—the anchor arm—captured nucleic acid—the genome-informed arm—the second unique targeting molecular tag—a unique sample barcode—a second sequencing primer—a second sequencing adaptor.

71. The method of any one of claims 60-70, wherein the first plurality of target sequences of interest is on a single chromosome.

72. The method of any one of claims 60-71, wherein the second plurality of target sequences of interest are on multiple chromosomes.

73. The method of claim 63, wherein at least some of the nucleic acids of interest in the subject are cell-free target nucleic acids, the method further comprising:
a) measuring amounts of amplicons from the nucleic acid sample corresponding to each of a plurality of sizes, the amounts comprising amplicons from cell-free target nucleic acids and from background nucleic acids, thereby measuring amounts of nucleic acids at the plurality of sizes;
b) calculating a first value of a first parameter based on the amounts of nucleic acids at the plurality of sizes, the first parameter providing a statistical measure of a size profile of nucleic acids in the sample;
c) comparing the first value to a reference value; and
d) estimating the fractional concentration of the target nucleic acids among background nucleic acid in the sample based on the comparison of step c).

74. The method of claim 73, wherein the cell-free target nucleic acids are of fetal origin, and the background nucleic acids comprise maternal nucleic acids, whereby the concentration of fetal nucleic acids in a maternal sample is determined.

75. The method of claim 74, wherein the reference value is from one or more pregnant subjects with known concentrations of fetal nucleic acids.

76. The method of claim 73, wherein the cell-free target nucleic acids are from a tumor, and the background nucleic acids comprise non-tumor nucleic acids, whereby the concentration of tumor nucleic acids in a sample is determined.

77. The method of claim 76, wherein the reference value is from one or more cancer-free subjects.

78. The method of claim 73, wherein the cell-free target nucleic acids are from a donor, and the background nucleic acids comprise host nucleic acids, whereby the concentration of transplanted donor nucleic acids in a sample from the host is determined.

79. A method of determining the methylation status of one or more nucleic acid fragments in a subject comprising:
a) obtaining a nucleic acid sample isolated from a subject;
b) performing bisulfite conversion of the nucleic acid sample;
c) adding an anchor sequence to the bisulfite-converted nucleic acid of step b);
d) capturing a plurality of target sequences of interest in the nucleic acid sample obtained in step a) by using one or more populations of molecular inversion probes (MIPs) to produce a plurality of replicons,
wherein each of the MIPs in the population of MIPs comprises in sequence the following components:
anchor arm—polynucleotide linker—genome-informed arm;
wherein the anchor arm in each of the MIPs is substantially complementary to the anchor sequence from step c), and the genome-informed arm in each of the MIPs is substantially complementary to a repeat sequence in the nucleic acid, such that the anchor sequence and the repeat sequence flank a unique gap region in the plurality of target sequences of interest;
e) sequencing a plurality of MIP amplicons that are amplified from the replicons obtained in step d); and
f) determining the number of occurrences of cytosine nucleotides at each corresponding known CpG site within the MIP amplicons sequenced at step e), wherein the methylation status is determined based on the number of occurrences of cytosine nucleotides at each corresponding known CpG site.

80. The method of claim 79, wherein the size of one or more amplicons from the plurality of target sequences is determined based on the amplicon sequence from step d).

81. The method of claim 80, wherein at least some of the nucleic acids are cell-free target nucleic acids, the method further comprising:
a) measuring amounts of amplicons from the nucleic acid sample corresponding to each of a plurality of sizes, the amounts comprising amplicons from cell-free target nucleic acids and from background nucleic acids, thereby measuring amounts of nucleic acids at the plurality of sizes;
b) calculating a first value of a first parameter based on the amounts of nucleic acids at the plurality of sizes, the first parameter providing a statistical measure of a size profile of nucleic acids in the sample;
c) comparing the first value to a reference value; and
d) estimating the fractional concentration of the target nucleic acids among background nucleic acid in the sample based on the comparison of step c).

82. The method of claim 79, wherein the methylation status of step e), is compared to a reference value.

83. The method of claim 82, wherein the reference value is a methylation status from a specific tissue type.

84. The method of claim 83, wherein the reference value is a methylation status from a diseased tissue type.

85. A method for characterizing one or nucleic acids of interest from a subject, comprising:
a) obtaining a nucleic acid sample isolated from a subject;
b) adding clip sequences to the 3′ and 5′ ends of each of a plurality of target nucleic acids from the sample in step a) to create a clip product, wherein the two clip sequences flank a gap region in the target nucleic acid sequence of interest;
c) hybridizing a capture probe comprising two clip binding arms to the clip product of step b), wherein the two clip binding arms are on opposite ends of the same capture probe, and wherein each clip binding arm is substantially complementary one of the clip sequences from step b);
d) using a ligation/extension mixture to extend and ligate the gap region between the two clip binding arms to form a single-stranded circular nucleic acid molecule; and
e) analyzing a plurality of amplicons that are amplified from single-stranded circular nucleic acid molecules of step d) to characterize the one or more nucleic acids of interest.

86. The method of claim 85, wherein analyzing a plurality of amplicons of step e) comprises determining one or more of size, size distribution, nucleotide sequence, and/or amounts one or more of said plurality of amplicons.

87. The method of claim 85 or 86, wherein amplifying the plurality of amplicons from single-stranded circular nucleic acid molecules comprises a polymerase chain reaction.

88. The method of any one of claims 85-87, wherein the plurality of target nucleic acids are double-stranded, and wherein one or both clip sequences are added by ligation.

89. The method of claim 88, wherein the double-stranded plurality of nucleic acids is subjected to one or more of end-repair, phosphorylation, and A-tailing prior to ligation of said one or both clip sequences.

90. The method of any one of claims 85-89, wherein the clip sequences added in step a) are added using target-specific adaptor oligonucleotides, wherein the target-specific adapter oligonucleotides comprise a sequence substantially complementary to a clip arm and a sequence substantially complementary to a 5′ or 3′ terminal portion of a target nucleic acid sequence of interest.

91. The method of any one of claims 85-90, further comprising prior to step c) a step of treating the clip product with bisulfate under conditions wherein unmethylated cytosines are converted to uracils.

92. The method of claim 91, wherein the clip sequences added in step b) do not comprise cytosines.

93. The method of any one of claims 85-92, wherein the method further comprises an exonuclease digestion step that digests non-circular, linear nucleic acids.

94. The method of claim 93, wherein, following the exonuclease digestion, the method further comprises a linearizing step wherein the single-stranded circular nucleic acid molecule is cleaved to become linear.

95. The method of any one of claims 86-94, wherein nucleotide sequences of at least 50,000 different nucleic acids from the subject are determined using a single capture probe.

96. The method of any one of claims 86-95, wherein the sizes of at least 1,000 different nucleic acids in the nucleic acid sample are determined using a single capture probe.
